SEQUENCE LISTING (83 sequences)

SEQ ID NO: 1 - protein, 13 aa, Artificial Sequence
  Description of Artificial Sequence Synthetic peptide
LGGIFEAMKMVLH

SEQ ID NO: 2 - protein, 6 aa, Artificial Sequence
  Description of Artificial Sequence Synthetic peptide
LGGIFE

SEQ ID NO: 3 - protein, 7 aa, Artificial Sequence
  Description of Artificial Sequence Synthetic peptide
AMKMVLH

SEQ ID NO: 4 - protein, 11 aa, Artificial Sequence
  Description of Artificial Sequence Synthetic peptide
MASMTGGQQMG

SEQ ID NO: 5 - protein, 6 aa, Artificial Sequence
  Description of Artificial Sequence Synthetic peptide
MASMTG

SEQ ID NO: 6 - protein, 5 aa, Artificial Sequence
  Description of Artificial Sequence Synthetic peptide
GQQMG

SEQ ID NO: 7 - protein, 10 aa, Artificial Sequence
  Description of Artificial Sequence Synthetic peptide
SSTSSDFRDR

SEQ ID NO: 8 - protein, 9 aa, Artificial Sequence
  Description of Artificial Sequence Synthetic peptide
YPYDVPDYA

SEQ ID NO: 9 - protein, 15 aa, Artificial Sequence
  Description of Artificial Sequence Synthetic peptide
KETAAAKFERQHMDS

SEQ ID NO: 10 - protein, 9 aa, Artificial Sequence
  Description of Artificial Sequence Synthetic peptide
NANNPDWDF

SEQ ID NO: 11 - protein, 11 aa, Artificial Sequence
  Description of Artificial Sequence Synthetic peptide
MASMTGGQQMG

SEQ ID NO: 12 - protein, 12 aa, Artificial Sequence
  Description of Artificial Sequence Synthetic peptide
GKPIPNPLLGLD

SEQ ID NO: 13 - protein, 8 aa, Artificial Sequence
  Description of Artificial Sequence Synthetic peptide
DYKDDDDK

SEQ ID NO: 14 - protein, 6 aa, Artificial Sequence
  Description of Artificial Sequence Synthetic peptide
HHHHHH

SEQ ID NO: 15 - protein, 10 aa, Artificial Sequence
  Description of Artificial Sequence Synthetic peptide
QQKLISEEDL

SEQ ID NO: 16 - protein, 11 aa, Artificial Sequence
  Description of Artificial Sequence Synthetic peptide
YTDIEMNRLGK

SEQ ID NO: 17 - protein, 10 aa, Artificial Sequence
  Description of Artificial Sequence Synthetic peptide
WAEYCGPCKM

SEQ ID NO: 18 - protein, 6 aa, Artificial Sequence
  Description of Artificial Sequence Synthetic peptide
DTYRYI

SEQ ID NO: 19 - protein, 5 aa, Artificial Sequence
  Description of Artificial Sequence Synthetic peptide
DDDDK

SEQ ID NO: 20 - protein, 6 aa, Artificial Sequence
  Description of Artificial Sequence Synthetic peptide
TDFYLK

SEQ ID NO: 21 - protein, 13 aa, Artificial Sequence
  Description of Artificial Sequence Synthetic peptide
EAPVPYPDPLEPR

SEQ ID NO: 22 - protein, 12 aa, Artificial Sequence
  Description of Artificial Sequence Synthetic peptide
SQPELAPEDDED

SEQ ID NO: 23 - protein, 12 aa, Artificial Sequence
  Description of Artificial Sequence Synthetic peptide
EETARFQPGYRS

SEQ ID NO: 24 - protein, 11 aa, Artificial Sequence
  Description of Artificial Sequence Synthetic peptide
KPPTPPPEPET

SEQ ID NO: 25 - protein, 8 aa, Artificial Sequence
  Description of Artificial Sequence Synthetic peptide
NWSHPQFE

SEQ ID NO: 26 - protein, 6 aa, Artificial Sequence
  Description of Artificial Sequence Synthetic peptide
HTTPHH

SEQ ID NO: 27 - protein, 11 aa, Artificial Sequence
  Description of Artificial Sequence Synthetic peptide
YTDIEMNRLGK

SEQ ID NO: 28 - protein, 4 aa, Artificial Sequence
  Description of Artificial Sequence Synthetic peptide
DDDK

SEQ ID NO: 29 - protein, 6 aa, Artificial Sequence
  Description of Artificial Sequence Synthetic peptide
EYMPME

SEQ ID NO: 30 - protein, 12 aa, Artificial Sequence
  Description of Artificial Sequence Synthetic peptide
EDQVDPRLIDGK

SEQ ID NO: 31 - protein, 11 aa, Artificial Sequence
  Description of Artificial Sequence Synthetic peptide
YPYDVPDYASL

SEQ ID NO: 32 - protein, 10 aa, Artificial Sequence
  Description of Artificial Sequence Synthetic peptide
ETTYNTTLKY

SEQ ID NO: 33 - protein, 9 aa, Artificial Sequence
  Description of Artificial Sequence Synthetic peptide
TETSQVAPA

SEQ ID NO: 34 - protein, 12 aa, Artificial Sequence
  Description of Artificial Sequence Synthetic peptide
MKDGEEYSRAFR

SEQ ID NO: 35 - protein, 17 aa, Artificial Sequence
  Description of Artificial Sequence Synthetic peptide
CKETAAAKFERQHMDSC

SEQ ID NO: 36 - protein, 7 aa, Artificial Sequence
  Description of Artificial Sequence Synthetic peptide
CKETAAA

SEQ ID NO: 37 - protein, 10 aa, Artificial Sequence
  Description of Artificial Sequence Synthetic peptide
KFERQHMDSC

SEQ ID NO: 38 - protein, 11 aa, Artificial Sequence
  Description of Artificial Sequence Synthetic peptide
MASMTCGQQMG

SEQ ID NO: 39 - protein, 11 aa, Artificial Sequence
  Description of Artificial Sequence Synthetic peptide
MASMTGCQQMG

SEQ ID NO: 40 - protein, 11 aa, Artificial Sequence
  Description of Artificial Sequence Synthetic peptide
MASMTGGCQMG

SEQ ID NO: 41 - protein, 11 aa, Artificial Sequence
  Description of Artificial Sequence Synthetic peptide
MASMTGGQCMG

SEQ ID NO: 42 - protein, 12 aa, Artificial Sequence
  Description of Artificial Sequence Synthetic peptide
GGNWCHPQFEGG

SEQ ID NO: 43 - protein, 12 aa, Artificial Sequence
  Description of Artificial Sequence Synthetic peptide
GGNWSCPQFEGG

SEQ ID NO: 44 - protein, 12 aa, Artificial Sequence
  Description of Artificial Sequence Synthetic peptide
GGNWSHCQFEGG

SEQ ID NO: 45 - protein, 12 aa, Artificial Sequence
  Description of Artificial Sequence Synthetic peptide
GGNWSHPCFEGG

SEQ ID NO: 46 - protein, 12 aa, Artificial Sequence
  Description of Artificial Sequence Synthetic peptide
GGNWSHPQFEGG

SEQ ID NO: 47 - protein, 15 aa, Artificial Sequence
  Description of Artificial Sequence Synthetic peptide
KETAAAKFCRQHMDS

SEQ ID NO: 48 - protein, 15 aa, Artificial Sequence
  Description of Artificial Sequence Synthetic peptide
KETAAAKFGRQHMDS

SEQ ID NO: 49 - protein, 10 aa, Artificial Sequence
  Description of Artificial Sequence Synthetic peptide
MASMTGQQMG

SEQ ID NO: 50 - protein, 5 aa, Artificial Sequence
  Description of Artificial Sequence Synthetic peptide
MASMT

SEQ ID NO: 51 - protein, 7 aa, Artificial Sequence
  Description of Artificial Sequence Synthetic peptide
MASMTGG

SEQ ID NO: 52 - protein, 8 aa, Artificial Sequence
  Description of Artificial Sequence Synthetic peptide
MASMTGGQ

SEQ ID NO: 53 - protein, 4 aa, Artificial Sequence
  Description of Artificial Sequence Synthetic peptide
GGNW

SEQ ID NO: 54 - protein, 5 aa, Artificial Sequence
  Description of Artificial Sequence Synthetic peptide
GGNWS

SEQ ID NO: 55 - protein, 6 aa, Artificial Sequence
  Description of Artificial Sequence Synthetic peptide
GGNWSH

SEQ ID NO: 56 - protein, 7 aa, Artificial Sequence
  Description of Artificial Sequence Synthetic peptide
GGNWSHP

SEQ ID NO: 57 - protein, 8 aa, Artificial Sequence
  Description of Artificial Sequence Synthetic peptide
KETAAAKF

SEQ ID NO: 58 - protein, 6 aa, Artificial Sequence
  Description of Artificial Sequence Synthetic peptide
CGQQMG

SEQ ID NO: 59 - protein, 8 aa, Artificial Sequence
  Description of Artificial Sequence Synthetic peptide
CHPQFEGG

SEQ ID NO: 60 - protein, 7 aa, Artificial Sequence
  Description of Artificial Sequence Synthetic peptide
CPQFEGG

SEQ ID NO: 61 - protein, 7 aa, Artificial Sequence
  Description of Artificial Sequence Synthetic peptide
CRQHMDS

SEQ ID NO: 62 - DNA, 35 bp, Artificial Sequence
  Description of Artificial Sequence Synthetic oligonucleotide
ttggtgctcgagtcccccccccccccccccccccc

SEQ ID NO: 63 - DNA, 35 bp, Artificial Sequence
  Description of Artificial Sequence Synthetic oligonucleotide
ccccccccccccccccccccgctgccatcgatggt

SEQ ID NO: 64 - DNA, 35 bp, Artificial Sequence
  Description of Artificial Sequence Synthetic oligonucleotide
ccccccccccccccccccgtgccatccatagtcag

SEQ ID NO: 65 - DNA, 37 bp, Artificial Sequence
  Description of Artificial Sequence Synthetic oligonucleotide
ttggtgctcgagtcccccccgtgccatccatagtcag

SEQ ID NO: 66 - DNA, 28 bp, Artificial Sequence
  Description of Artificial Sequence Synthetic oligonucleotide
ggactcgagcaccaatactataaattcg

SEQ ID NO: 67 - DNA, 28 bp, Artificial Sequence
  Description of Artificial Sequence Synthetic oligonucleotide
cgaatttatactgaccatcgatggcagc

SEQ ID NO: 68 - DNA, 28 bp, Artificial Sequence
  Description of Artificial Sequence Synthetic oligonucleotide
cgaatttatactgactatggatggcacg

SEQ ID NO: 69 - protein, 8 aa, Artificial Sequence
  Description of Artificial Sequence Synthetic peptide
WSHPQFEG

SEQ ID NO: 70 - protein, 6 aa, Artificial Sequence
  Description of Artificial Sequence Synthetic peptide
HPQFEG

SEQ ID NO: 71 - protein, 6 aa, Artificial Sequence
  Description of Artificial Sequence Synthetic peptide
GGQQMG

SEQ ID NO: 72 - protein, 5 aa, Artificial Sequence
  Description of Artificial Sequence Synthetic peptide
CQQMG

SEQ ID NO: 73 - DNA, 28 bp, Artificial Sequence
  Description of Artificial Sequence Synthetic oligonucleotide
ccaattaatactgactatggatggcacg

SEQ ID NO: 74 - DNA, 28 bp, Artificial Sequence
  Description of Artificial Sequence Synthetic oligonucleotide
ccaattaatactgactatggatggcacg

SEQ ID NO: 75 - DNA, 27 bp, Artificial Sequence
  Description of Artificial Sequence Synthetic oligonucleotide
ggactcgagcaccaatactataaattc

SEQ ID NO: 76 - DNA, 26 bp, Artificial Sequence
  Description of Artificial Sequence Synthetic oligonucleotide
ggactcgagcaccaatactataaatt

SEQ ID NO: 77 - DNA, 27 bp, Artificial Sequence
  Description of Artificial Sequence Synthetic oligonucleotide
gaatttatactgactatggatggcacg

SEQ ID NO: 78 - DNA, 26 bp, Artificial Sequence
  Description of Artificial Sequence Synthetic oligonucleotide
aatttatactgactatggatggcacg

SEQ ID NO: 79 - protein, 4 aa, Artificial Sequence
  Description of Artificial Sequence Synthetic peptide
CQMG

SEQ ID NO: 80 - protein, 3 aa, Artificial Sequence
  Description of Artificial Sequence Synthetic peptide
CMG

SEQ ID NO: 81 - protein, 6 aa, Artificial Sequence
  Description of Artificial Sequence Synthetic peptide
CQFEGG

SEQ ID NO: 82 - protein, 5 aa, Artificial Sequence
  Description of Artificial Sequence Synthetic peptide
CFEGG

SEQ ID NO: 83 - protein, 4 aa, Artificial Sequence
  Description of Artificial Sequence Synthetic peptide
QQMG

(19) United States
(12) Patent Application Publication
(10) Pub. No.: US 2010/0159446 A1
(43) Pub. Date: Jun. 24, 2010

(54) Detection Assays and Use Thereof

(76) Inventors: Lawrence A. Haff, Westborough, MA (US); Yumei Huang, North Billerica, MA (US); Richard A. Martinelli, Belmont, MA (US); Benjamin A. Seigal, Watertown, MA (US); David J. Livingston, Barrington, RI (US); Wei-Chuan Sun, Acton, MA (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/176,798

(22) Filed: Jul. 21, 2008

Related U.S. Application Data
(60) Provisional application No. 60/962,333, filed on Jul. 27, 2007.

(51) Int. Cl. C12Q 1/68

(52) U.S. Cl. 435/6

(57) ABSTRACT

The invention provides compositions and methods for the detection and/or quantification of biological targets (e.g., nucleic acids and proteins) by the nucleic acid-templated creation of one or more reaction products, for example, epitopes, enzyme substrates, enzyme activators, and ligands. The reaction products can be detected and/or quantitated after signal amplification using an amplification system.

### CROSS REFERENCE TO RELATED APPLICATION

This application claims the benefit of and priority to U.S. Provisional Patent Application No. 60/962,333, filed Jul. 27, 2007, the entire disclosure of which is incorporated herein by reference.

### FIELD OF THE INVENTION

The invention relates generally to assay technologies and their use in biodetection and diagnostics. More particularly, the invention relates to compositions and methods of nucleic acid-templated chemistry (e.g., synthesis of reaction products) in biodetection and diagnostics.

### BACKGROUND

The principle of detection based upon a target-dependent DNA-programmed chemistry (“DPC”) reaction has been demonstrated, for example, in WO06128138A2 by Coull et al. For certain applications, DPC reactions may create a single detectable molecule, for example, a fluorophore, per target molecule. This can provide assays with the desired sensitivity. However, certain other assays may lack the requisite sensitivity. For example, in certain assays, the production of a single detectable molecule may not confer the system with adequate sensitivity to detect a biological target, for example, a protein dimer present at low levels, in tissue or body fluid samples.

Accordingly, there is an ongoing need to provide assay systems with improved detection sensitivities to permit the detection of certain biological targets, for example, proteins or nucleic acids, in samples of interest.

### SUMMARY OF THE INVENTION

The present invention is based, in part, upon the discovery that improved detection limits in DNA programmed chemistry (DPC)-mediated assays may be achieved if a plurality of detectable moieties can be produced per target molecule. In essence, a DPC-mediated reaction is employed to detect a target molecule via the production of one or more reaction products. Each molecule of reaction product then is used to produce a plurality of detectable moieties using amplification methodologies. As a result, the sensitivity of a given assay can be increased to permit the detection and/or quantification of a biological target in a sample, for example, a tissue or body fluid sample.

Depending upon the assay format chosen, the reaction product can be, for example, an intact epitope, enzyme substrate, enzyme activator or ligand, each of which may be detected or quantified by using direct or indirect detection systems, which are discussed in more detail hereinbelow. The detection systems employed in this invention comprise a detection component and an amplification component that interact with one another to amplify the signal resulting from the DPC reaction thereby increasing the sensitivity of the assay. For example, as discussed in more detail below, when the reaction product is an intact epitope, the epitope can be recognized by an antibody. The antibody can be associated (for example, covalently associated) with any one of several commonly employed signal-generating systems, such as, an enzyme, such as alkaline phosphate or peroxidase (Tijssen, P. “Practice and Theory of Enzyme Immunoassay”, in Laboratory Techniques in Biochemistry and Molecular Biology, vol. 15, 1985, R. H. Burdon and P. H. van Knippenberg, eds., Elsevier, Amsterdam). Alternatively, the antibody that binds to the epitope can be unlabelled. In this case, the unlabelled antibody is then bound by another antibody or other binding moiety associated (for example, covalently associated) with a signal-generating system. When enzymes are employed they have high turnover rates and can quickly produce large amounts of detectable moieties from starting substrates, for example, colorimetric, fluorescent, and chemiluminescent precursor substrates.

In one aspect, the invention provides a method of determining the presence and/or amount of a biological target in a sample. The method comprises combining the sample with (1) a first probe comprising (i) a first binding moiety with binding affinity to the biological target, (ii) a first oligonucleotide sequence associated (for example, covalently or non-covalently associated) with the first binding moiety, and (iii) a first product precursor associated (for example, covalently or non-covalently associated) with the first oligonucleotide sequence, and (2) a second probe comprising (i) a second binding moiety with binding affinity to the biological target, (ii) a second oligonucleotide sequence associated (for example, covalently or non-covalently associated) to the second binding moiety and capable of hybridizing to the first oligonucleotide sequence, and (iii) a second product precursor associated (for example, covalently or non-covalently associated) with the second oligonucleotide sequence, under conditions to permit both the first and second binding moieties to bind to the biological target, if present in the sample. When the first and second binding moieties bind to the biological target, the first and second oligonucleotide sequences hybridize to one another to bring the first and second product precursors into reactive proximity with one another to produce a reaction product. The reaction product can be an intact epitope, an enzyme substrate, an enzyme activator or a ligand.

The resulting reaction product, if present, is exposed to a detection system comprising a detection component capable of interacting with the reaction product and an amplification component capable of producing a plurality of detectable moieties. The presence and/or amount of the detectable moieties is indicative of the presence and/or amount of the biological target in the sample.

It is understood that the first probe and the second probe can each be a single molecule. For example, in the first and second probes, the binding moiety can be covalently associated with the product precursor via one or more oligonucleotide sequences. Alternatively, the first probe and the second probe can comprise two or more pieces that interact with one another to produce a functional probe. This can be facilitated, for example, through a zipcode oligonucleotide sequence covalently associated with the binding moiety that is hybridized to a complementary or substantially complementary anti-zipcode oligonucleotide sequence covalently associated with product precursor. The probe also includes one or more oligonucleotides that in certain embodiments are covalently associated at one end to the antizip oligonucleotide sequence and at the other end to the product precursor.

In another aspect, the invention provides a method for determining the presence and/or amount of a biological target in a sample. The method comprises:

(a) providing a first target binding component comprising (i) a first binding moiety having binding affinity to the biological target, and (ii) a first oligonucleotide zipcode sequence associated (for example, covalently or non-covalently associated) to the first binding moiety;

(b) providing a second target binding component comprising (i) a second binding moiety having binding affinity to the biological target, and (ii) a second oligonucleotide zipcode sequence associated (for example, covalently or non-covalently associated) to the second binding moiety;

(c) providing a first reporter component comprising (i) a first oligonucleotide anti-zipcode sequence capable of hybridizing to the first oligonucleotide zipcode sequence, (ii) a first reporter oligonucleotide associated (for example, covalently or non-covalently associated) to the first oligonucleotide anti-zipcode sequence, and (iii) a first product precursor associated (for example, covalently or non-covalently associated) with the first reporter oligonucleotide; and

(d) providing a second reporter component comprising (i) a second oligonucleotide anti-zipcode sequence capable of hybridizing to the second oligonucleotide zipcode sequence, (ii) a second reporter oligonucleotide oligonucleotide associated (for example, covalently or non-covalently associated) with the second oligonucleotide anti-zipcode sequence and capable of hybridizing to the first reporter oligonucleotide sequence, and (iii) a second product precursor associated (for example, covalently or non-covalently associated) with the second reporter oligonucleotide sequence and capable of reacting with the first product precursor when brought into reactive proximity.

In one embodiment, the sample is simultaneously combined with the first target binding component, the second target binding component, the first reporter component, and the second reporter component under conditions so that the first and second binding moieties bind to the biological target, if present in the sample. Once the first and second binding moieties bind to the biological target, the first zipcode sequence hybridizes to the first anti-zipcode oligonucleotide sequence, the second oligonucleotide zipcode sequence hybridizes to the second oligonucleotide anti-zipcode sequence, and the second reporter oligonucleotide hybridizes to the first reporter oligonucleotide to bring the first and second reaction product precursors into reactive proximity to produce a reaction product.

In another embodiment, the first target binding component, the second target binding component, the first reporter component, and the second reporter are pre-incubated with one another under conditions to permit to the first and second oligonucleotide zipcode sequences to anneal to the corresponding first and second oligonucleotide anti-zipcodes sequences to produce functional probes before they are combined with the sample. It is understood that the order of additions can be varied to optimize the signal-to-noise ratio. For example, the first and second target binding components can be incubated with the sample and permitted to bind to the biological target before the first and second reporter components are added.

The resulting reaction product, if any, is exposed to a detection system. Thereafter, the presence and/or amount of the detectable moieties can be used to determine the presence and/or amount of the biological target in the sample.

In each of the methods described herein, the amplification component of the detection system comprises a catalyst, for example, an enzyme, that catalyzes the production of the detectable moieties. For example, the amplification component can produce at least 10, 100, 1,000, or 10,000 molecules of the detectable moieties per molecule of the reaction product. Certain exemplary enzymes include, for example, peroxidases, for example, horseradish peroxidase (HRP), phosphatases, for example, alkaline phosphatase, nucleases, for example, ribonuclease, and dehydrogenases, for example, lactate dehydrogenase.

Depending upon the assay format chosen, the reaction product can be a peptide or protein. For example, the reaction product can comprise one or more of the peptidyl sequences disclosed herein, for example, the peptidyl sequences discussed hereinbelow in Example 3 as well as those appearing, for example, in FIG. 15. Alternatively, the reaction product can be a small molecule, for example, a small molecule that defines an epitope. The reaction product can be a dye, antibiotic, enzyme cofactor, enzyme inhibitor, pesticide, drug, toxin, fluorophore, chromophore, hormone, carbohydrate or lipid.

It is understood that the methods described herein can be used to detect and/or quantify a number of biological targets, which can include, for example, a protein or peptide. The methods can be used to determine the presence and/or amount of multimeric proteins, for example, homodimeric proteins, heterodimeric proteins, and fusion proteins. Exemplary biological targets can include, for example, a Bcr-Abl heterodimer, an ErbB family homodimer, an ErbB family heterodimer, and PDGF. Alternatively, the methods described herein can be used to detect and/or quantify a nucleic acid, for example, a DNA or an RNA.

Depending upon the biological target and assay format, the first and second binding moieties can each bind to separate binding sites defined by the biological target. Furthermore, the first and second binding moieties can be the same or different. Furthermore, the first binding moiety, the second binding moiety or each of first and second binding moieties can be an antibody.

Furthermore, depending upon the assay format and the DPC chemistries employed, the first product precursor and the second product precursor may react with one another only in the presence of an additional reagent, for example, a reagent needed to facilitate the chemical reaction. However, depending upon the chemistry chosen, the first product precursor may react spontaneously with the second product precursor to produce the reaction product. One such approach, as described herein, is referred to as native chemical ligation, wherein in one embodiment, for example, a peptide bond is produced by a reaction between a first precursor peptide containing a C-terminal thioester and a second precursor peptide containing an N-terminal cysteine. In certain embodiments, a peptide bond isostere is produced by a reaction between a C-terminal thioester and an N-terminal thiol that is provided by a moiety other than a cysteine. It is understood that it may be necessary to adjust certain reactants and reaction conditions to maximize assay specificity. This can be achieved, for example, by selecting the first and second oligonucleotide sequences or the first and second reporter oligonucleotide sequences to have a melting temperature of from about 8° C. to about 25° C., more preferably from about 9° C. to about 20° C. Alternatively or in addition, this can be achieved, for example, by incubating the sample with a probe comprising the first product precursor, removing unbound first product probe and then incubating the sample with the second probe comprising the second product precursor.

In another aspect, the invention provides another method of determining the presence and/or amount of a biological target in a sample based on the unmasking of a product precursor. The method comprises combining the sample with (1) a first probe comprising (i) a first binding moiety with binding affinity to the biological target, (ii) a first oligonucleotide sequence associated (for example, covalently or non-covalently associated) with the first binding moiety, and (iii) a first masked product precursor associated (for example, covalently or non-covalently associated) with the first oligonucleotide sequence and (2) a second probe comprising (i) a second binding moiety with binding affinity to the biological target, (ii) a second oligonucleotide sequence associated (for example, covalently or non-covalently associated) with the second binding moiety and capable of hybridizing to the first oligonucleotide sequence, and (iii) an unmasking group associated (for example, covalently or non-covalently associated) with the second oligonucleotide sequence, under conditions to permit the first and second binding moieties to bind to the biological target, if present in the sample. When the binding moieties bind to the biological target, the first and second oligonucleotide sequences hybridize to one another to bring the unmasking group into reactive proximity with the masked product precursor to produce a reaction product, namely an unmasked reaction product.

The resulting reaction product, if any, is exposed to a detection system. The presence and/or amount of the detectable moieties can then be used to determine the presence and/or amount of the biological target in the sample.

It is understood that the masked precursor can be a masked epitope, masked enzyme substrate, masked enzyme activator or a masked ligand. During the reaction, the masking group is removed to produce an unmasked product, for example, an unmasked epitope, unmasked enzyme substrate, unmasked enzyme activator or an unmasked ligand. The reaction product can be, for example, a peptide, protein or a small molecule.

Under certain circumstances, the biological target can be a multimeric protein, for example, a homodimeric protein, heterodimeric protein or fusion protein. For example, the biological target can be selected from the group consisting of a Bcr-Abl heterodimer, an ErbB family homodimer, an ErbB family heterodimer, and PDGF.

Depending upon the biological target and the assay format, the first binding moiety, the second binding moiety, or each of the first binding moiety and the second binding moiety can be an antibody. Furthermore, it is understood that the first and second binding moieties can be the same or different.

Furthermore, depending upon the amplification component chosen, the amplification component can comprise an enzyme that catalyzes the production of the detectable moieties. Depending upon the assay sensitivity required for a particular application, the amplification component may be capable of producing at least 10, 100, 1,000 or 10,000 molecules of the detectable moieties per molecule of reaction product.

In another aspect, the invention provides a kit to facilitate one or more of the assays described herein. The kit comprises a first probe comprising (i) a first binding moiety with binding affinity to a biological target, (ii) a first reporter oligonucleotide sequence associated (for example, covalently or non-covalently associated) with the first binding moiety, and (iii) a first product precursor associated (for example, covalently or non-covalently associated) with the first reporter oligonucleotide sequence. The kit also comprises a second probe comprising (i) a second binding moiety with binding affinity to the biological target, (ii) a second reporter oligonucleotide sequence associated (for example, covalently or non-covalently associated) with the second binding moiety, and (iii) a second product precursor associated (for example, covalently or non-covalently associated) with the second reporter oligonucleotide sequence, wherein upon the binding of the first and second binding moieties to the biological target the first and second reporter oligonucleotide sequences are capable of hybridizing to one another and the first and second product precursors are capable of reacting with one another to produce a reaction product. The reaction product can be selected from the group consisting of an intact epitope, an enzyme substrate, an enzyme activator, and a ligand.

In another aspect, the kit comprises a first probe comprising (i) a first binding moiety with binding affinity to a biological target, (ii) a first reporter oligonucleotide sequence associated (for example, covalently or non-covalently associated) with the first binding moiety, and (iii) a first masked product precursor associated (for example, covalently or non-covalently associated) with the first reporter oligonucleotide sequence. The kit also comprises a second probe comprising (i) a second binding moiety with binding affinity to the biological target, (ii) a second reporter oligonucleotide sequence associated (for example, covalently or non-covalently associated) with the second binding moiety, and (iii) an unmasking group associated (for example, covalently or non-covalently associated) with the second reporter oligonucleotide sequence. Upon the binding of the first and second binding moieties to the biological target the first and second reporter oligonucleotide sequences hybridize to one another and the unmasking agent and the masked product precursor react with one another to produce a reaction product (namely, an unmasked reaction product). The reaction product can be selected from the group consisting of an unmasked epitope, an unmasked enzyme substrate, an unmasked enzyme activator, and an unmasked ligand.

The kits described herein optionally also comprise a detection system capable of producing a plurality of detectable moieties. Furthermore, the kit optionally also comprises instructions for using the kit for detecting the biological target.

In certain of the kits, each of the first and second probes is a single molecule where the components of each probe are covalently associated with one another. Alternatively, each of the first and second probes can comprise a plurality of components that are non-covalently associated with one another to produce functional probes. For example, the probes can comprise two or more oligonucleotide sequences, for example, a zipcode oligonucleotide sequence and a complementary or substantially complementary anti-zipcode oligonucleotide sequence, which are capable of hybridizing to one another to permit non-covalent association of the various probe components.

In another aspect, the invention provides another kit to facilitate one or more of the assays described herein. The kit comprises,

(a) a first target binding component comprising (i) a first binding moiety having binding affinity to the biological target, and (ii) a first oligonucleotide zipcode sequence associated (for example, covalently or non-covalently associated) with the first binding moiety;

(b) a second target binding component comprising (i) a second binding moiety having binding affinity to the biological target, and (ii) a second oligonucleotide zipcode sequence associated (for example, covalently or non-covalently associated) with the second binding moiety;

(c) a first reporter component comprising (i) a first oligonucleotide anti-zipcode sequence capable of hybridizing to the first oligonucleotide zipcode sequence, (ii) a first reporter oligonucleotide associated (for example, covalently or non-covalently associated) with the first oligonucleotide zipcode sequence, and (iii) a first product precursor associated (for example, covalently or non-covalently associated) with the first reporter oligonucleotide; and

(d) a second reporter component comprising (i) a second oligonucleotide anti-zipcode sequence capable of hybridizing to the second oligonucleotide zipcode sequence, (ii) a second reporter oligonucleotide associated (for example, covalently or non-covalently associated) with the second oligonucleotide zipcode sequence and capable of hybridizing to the first reporter oligonucleotide sequence, and (iii) a second product precursor associated (for example, covalently or non-covalently associated) with the second reporter oligonucleotide sequence. Upon binding of the first and second binding moieties to the biological target and hybridization of the respective zipcode and anti-zipcode oligonucleotide sequences, the reporter oligonucleotide sequences hybridize to one another to bring the first and second product precursors into reactive proximity to produce a reaction product, for example, an intact epitope, an enzyme substrate, an enzyme activator, and a ligand.

The kits described herein optionally also comprise a detection system capable of producing a plurality of detectable moieties. Furthermore, the kit optionally also comprises instructions for using the kit for detecting the biological target.

Furthermore, in each of the kits, the reaction product can comprise a peptidyl sequence selected from MASMTGGQQMG (SEQ ID NO: 4), MASMTCGQQMG (SEQ ID NO: 38), MASMTGCQQMG (SEQ ID NO: 39), MASMTGGCQMG (SEQ ID NO: 40), MASMTGGQCMG (SEQ ID NO: 41), (G)₀₋₂-NWCHPQFE-(G)₀₋₂ (SEQ ID NO: 42), (G)₀₋₂-NWSCPQFE-(G)₀₋₂ (SEQ ID NO: 43), (G)₀₋₂-NWSHCQFE-(G)₀₋₂ (SEQ ID NO: 44), (G)₀₋₂-NWSHPCFE-(G)₀₋₂ (SEQ ID NO: 45), (G)₀₋₂-NWSHPQFE-(G)₀₋₂ (SEQ ID NO: 46), KETAAAKFCRQHMDS (SEQ ID NO: 47), KETAAAKFGRQHMDS (SEQ ID NO: 48), and MASMTG-[SCH₂C(O)]-QQMG (SEQ ID NO: 49). Furthermore, each of the kits can comprise an antibody that binds a biological target selected from the group consisting of Bcr-Abl, an ErbB family homodimer, an ErbB family heterodimer, and PDGF.

In another aspect, the invention provides molecules that can be used in the methods and kits described herein. The molecule can comprise a peptidyl portion selected from the group consisting of: MASMTCGQQMG (SEQ ID NO: 38), MASMT-thioester (SEQ ID NO: 50), MASMTGCQQMG (SEQ ID NO: 39), MASMTG-thioester (SEQ ID NO: 5), MASMTGGCQMG (SEQ ID NO: 40), MASMTGG-thioester (SEQ ID NO: 51), MASMTGGQCMG (SEQ ID NO: 41), MASMTGGQ-thioester (SEQ ID NO: 52), (G)₀₋₂-NWCHPQFE-(G)₀₋₂ (SEQ ID NO: 42), (G)₀₋₂-NW-thioester (SEQ ID NO: 53), (G)₀₋₂-NWSCPQFE-(G)₀₋₂ (SEQ ID NO: 43), (G)₀₋₂-NWS-thioester (SEQ ID NO: 54), (G)₀₋₂-NWSHCQFE-(G)₀₋₂ (SEQ ID NO: 44), (G)₀₋₂-NWSH-thioester (SEQ ID NO: 55), (G)₀₋₂-NWSHPCFE-(G)₀₋₂ (SEQ ID NO: 45), (G)₀₋₂-NWSHP-thioester (SEQ ID NO: 56), KETAAAKFCRQHMDS (SEQ ID NO: 47), KETAAAKF-thioester (SEQ ID NO: 57), CGQQMG (SEQ ID NO: 58), CHPQFE-(G)₀₋₂ (SEQ ID NO: 59), CPQFE-(G)₀₋₂ (SEQ ID NO: 60) CRQHMDS (SEQ ID NO: 61), and MASMTG-[SCH₂C(O)]-QQMG (SEQ ID NO: 49). In the foregoing peptide sequences, a thioester has the formula —C(O)—S—R, wherein R is any moiety that does not inhibit the formation of a peptide bond between a peptide containing a C-terminal thioester and a peptide containing an N-terminal cysteine, for example, a C₁-C₆ straight or branched alkyl. Furthermore, in the peptide of SEQ ID NO: 49, the group [SCH₂C(O)] refers to a linker where each of the components represent atoms (e.g., “S” is a sulfur) rather than amino acids.

### DEFINITIONS

The term “antibody,” as used herein, refers to an intact antibody (for example, a monoclonal antibody or an intact antibody found in polyclonal antisera), an antigen binding fragment of an antibody, or a biosynthetic antibody binding site. Antibody fragments include, for example, Fab, Fab′, (Fab′)₂ or Fv fragments. The antibodies and antibody fragments can be produced using conventional techniques known in the art. A number of biosynthetic antibody binding sites are known in the art and include, for example, single Fv or sFv molecules, described, for example, in U.S. Pat. Nos. 5,091,513, 5,132,405, and 5,476,786. Other biosynthetic antibody binding sites include, for example, bispecific or bifunctional binding proteins, for example, bispecific or bifunctional antibodies, which are antibodies or antibody fragments that bind at least two different epitopes. Methods for making bispecific antibodies are known in art and, include, for example, by fusing hybridomas or by linking Fab′ fragments. See, e.g., Songsivilai et al. (1990) CLIN. EXP. IMMUNOL. 79: 315-325; Kostelny et al. (1992) J. IMMUNOL. 148: 1547-1553.

The term “associated with,” as used herein, refers to an interaction between or among two or more groups, moieties, compounds, monomers, polymers, or small molecules. The interaction unless specified herein, can include both covalent and non-covalent associations. Covalent associations may occur through, for example, an amide, ester, carbon-carbon, disulfide, carbamate, ether, or carbonate linkage. Non-covalent associations may include, for example, hydrogen bonding, van der Waals interactions, hydrophobic interactions, magnetic interactions, and electrostatic interactions. Non-covalent interactions specifically include oligonucleotide hybridization. The term also includes attachment through a spacer or cross-linker, such as, an oligonucleotide linker sequence, a peptide linker sequence, a chemical linker, or any functional equivalent of the foregoing, or any combination thereof.

The term “binding moiety,” as used herein, refers to one molecule that is capable of binding specifically to a different molecule. Exemplary, binding moieties include, for example, proteins (for example, antibodies, adnectins, affibodies, receptors, ligands, growth factors, hormones, cytokines, avidin and avidin analogs), nucleic acids (for example, single stranded DNA or RNA sequences, aptamers), carbohydrates, lipids, and small molecules.

The term “detection system,” as used herein, refers to a system containing one or more components that permit the detection of a reaction product (including unmasked reaction products) and synthesis of a plurality of detectable moieties (e.g., moieties that can be detected either visually or with a suitable detector, e.g., optical detector, fluorescence detector, colorimeter, isotope detector) from a single reaction product. The detection system comprises a detection component and an amplification component. The detection component interacts preferentially with the reaction products versus the product precursors or masked product precursors and, therefore, produces significantly more detectable moieties when exposed to the reaction product than when exposed to product precursors or masked product precursors. For example, the number of detectable moieties produced when the detection component and the amplification component interact with the product precursors and/or the masked product precursors is less than 20%, less than 10%, less than 5%, less than 1%, or less than 0.1% of those produced in the presence of the reaction product.

The term “detection component,” as used herein, refers to a component of the detection system that interacts preferentially with and/or binds preferentially to the reaction product (including an unmasked reaction product) rather than a product precursor or a masked product precursor. The detection component can be, for example, a binding moiety, for example, an antibody, an affibody, a ligand, receptor, aptamer, adnectin, enzyme, or small molecule (for example, avidin or streptavidin).

The term “amplification component,” as used herein, refers to a component of the detection system that associates directly or indirectly with the detection component to produce a plurality of detectable moieties. The amplification component can be part of the same molecule as the detection component, for example, enzyme component of an anti-reaction product antibody-enzyme (e.g., HRP) conjugate. Alternatively, the amplification component and the detection component can be different molecules that interact with one another, for example, an enzyme component of an anti-detection component antibody-enzyme (e.g., HRP) conjugate that binds to the detection component, wherein the detection component binds to the reaction product. The amplification component can comprise two or more molecules that act together or interact with one another to produce the detectable moieties. For example, the amplification component can include precursors of the detectable moieties that are converted into detectable moieties by other agents of the amplification system.

The terms, “DNA programmed chemistry,” “DPC,” “nucleic acid programmed chemistry” or “nucleic acid-templated reactions” as used herein, are synonymous and refer to chemical reactions where nucleic acid sequences control the reactivity of reactants associated therewith to produce specific reaction products. In general, the reactions are accomplished by (i) providing one or more nucleic acid templates, which have associated reactive group(s); (ii) providing one or more reagents (sometimes referred to as transfer units) having an oligonucleotide sequence complementary sequence to at least a portion of the one or more templates and associated reactive group(s); and (iii) contacting the template and reagents under conditions to allow the reagents (via their complementary oligonucleotide sequences) to hybridize to the template and to bring the reactive groups into reactive proximity to yield one or more reaction products. For example, in a one-step nucleic acid-templated reaction, hybridization of a “template” and a “complementary” oligonucleotide bring the reactive groups associated therewith into reactive proximity to permit a chemical reaction that produces a particular product. Structures of the reactants and products need not be related to those of the nucleic acid sequences present in the template and the reagents. For a discussion of nucleic acid-templated reactions, see, e.g., U.S. Pat. Nos. 7,070,928 B1 and 7,223,545 and European Patent No. 1,423,400 B1 by Liu et al.; U.S. Patent Publication No. 2004/0180412 (U.S. Ser. No. 10/643,752; Aug. 19, 2003) by Liu et al., by Liu et al.; Gartner, et al., (2004), Science, vol. 305, pp. 1601-1605; Doyon, et al., (2003), JACS, vol. 125, pp. 12372-12373, all of which are incorporated by reference herein. See, also, “Turn Over Probes and Use Thereof” by Coull et al., PCT WO07/008,276A2, filed on May 3, 2006.

The term “epitope,” as used herein, refers to a molecule or a portion of a molecule (for example, a biomolecule) or small molecule that is recognized and bound by an epitope-binding molecule, such as, an antibody. Classically, an epitope is a small part of a macromolecule, often part of a protein, which is recognized by an antibody. For certain epitopes, the epitope may be defined by a linear sequence of amino acids or may result from amino acids brought into proximity with one another via the three-dimensional structure of a portion of the molecule that defines the epitope. Epitopes are frequently peptide sequences. The term “epitope,” as used herein also refers to a small molecule of any type, or a portion thereof, including a peptide, which may not be immunogenic by itself, but when coupled to a macromolecule, such as, a protein other than an antibody, will elicit an immune response specific to either the small molecule, the macromolecule or the small molecule/macromolecule complex. Antibodies are now commonly available that bind to a wide variety of epitopes consisting of small molecules, such as, hormones, drugs, pesticides and toxins, and such antibodies are frequently employed in detection assays for these small molecules.

The terms, “nucleic acid”, “oligonucleotide” (sometimes simply referred to as “oligo”) or “polynucleotide,” as used herein, refer to a polymer of nucleotides. The polymer may include, without limitation, natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages). Nucleic acids and oligonucleotides may also include other polymers of bases having a modified backbone, such as a locked nucleic acid (LNA), a peptide nucleic acid (PNA), a threose nucleic acid (TNA).

The terms, “protein” and “peptide,” as used herein, refer to a polymer of amino acids and does not refer to a specific length or number of amino acids. It is understood that the amino acids can be naturally or non-naturally occurring and can contain one or more modifications, for example, one or more modifications to an amino acid side chain. Furthermore, the polymer can contain one or more peptidyl bonds and optionally one or more modified linkages.

The terms “product precursor,” or “reaction product precursor” as used herein refer to any atom or molecule that is present in a starting material that is converted into a reaction product by DNA programmed chemistry. It is understood that the entire product precursor or reaction product precursor, or a portion thereof can be present in the reaction product. Precursors can include, for example, a portion of a small molecule, enzyme substrate, enzyme activator, ligand or epitope.

The terms “masked precursor” or “masked product precursor,” as used herein, refer to any molecule that has been inactivated by one or more chemical groups, which when removed can result in an operative product, for example, an operative enzyme substrate, operative enzyme activator, operative ligand and operative epitope.

The term “small molecule,” as used herein, refers to an organic compound either synthesized in the laboratory or found in nature having a molecular weight less than 5,000 grams per mole, optionally less than 2,000 grams per mole, and optionally less than 1,000 grams per mole.

Throughout the description, where compositions are described as having, including, or comprising specific components, or where processes are described as having, including, or comprising specific process steps, it is contemplated that compositions of the present invention also consist essentially of, or consist of, the recited components, and that the processes of the present invention also consist essentially of, or consist of, the recited processing steps. Further, it should be understood that the order of steps or order for performing certain actions are immaterial so long as the invention remains operable. Moreover, two or more steps or actions may be conducted simultaneously.

### BRIEF DESCRIPTION OF THE FIGURES

The invention may be further understood from the following figures in which:

FIG. 1 is a schematic representation of an exemplary method for the detection of a biological target via DPC-based epitope creation. The assay uses two probes (denoted ligand-reporter assemblies), where each assembly comprises a binding moiety (binding ligand) for a site on a biological target (denoted L₁ or L₂), a nucleic acid sequence (denoted reporter nucleic acid or complement), and a precursor molecule (denoted either Precursor 1 or Precursor 2) which is capable of a chemical reaction. Each ligand-reporter assembly may contain optional spacer groups (denoted Sp1, Sp2, Sp3, Sp4) and cross-linking groups (denoted CL). The reporter nucleic acid sequence and the complement are normally entirely or mostly self-complementary and anti-parallel in order to form a nucleic acid duplex. Once the ligand-reporter assemblies bind to the target, the reporter nucleic acid and complement hybridize to one another and bring Precursor 1 into reactive proximity to Precursor 2 to produce a product, for example, a product that contains an epitope. The reaction product can be detected by using an antibody (denoted AB) that binds to the epitope.

FIG. 2 is a schematic representation of a probe (a two-piece ligand-reporter assembly) that is produced from two separate oligonucleotide conjugates. One oligonucleotide conjugate (denoted target binding component) contains a binding moiety (L), optional spacer/crosslinker (Sp/CL), and a sequence of zipcode DNA. The other oligonucleotide conjugate (denoted reporter conjugate) contains a precursor, an optional spacer or crosslinker (Sp/CL), a reporter nucleic acid, an optional spacer (Sp) and a sequence of anti-zipcode. The zipcode (“zip”) and anti-zipcode (“antizip”) sequences are complementary or substantially complementary and are normally longer in sequence than the reporter nucleic acids, and their sequences are chosen so they do not anneal to reporter sequences. The zipcode and anti-zipcode sequences hybridize together to form a stable duplex which supports a stable ligand-reporter complex. The resulting complex is a functional equivalent of the single-molecule ligand-reporter assembly shown on FIG. 1.

FIG. 3 is a summary of exemplary DPC reactions that can generate an epitope.

FIG. 4 is a schematic representation of a method for removing a blocking azido group from the epsilon lysine in a substrate for the enzyme biotin ligase making this site available for biotinylation by the biotin ligase.

FIG. 5 is a schematic representation of the synthesis of the azido biotin ligase peptide (BLP)-oligonucleotide conjugate.

FIG. 6 is a schematic representation of an exemplary assay format that can detect the presence of a biotin molecule that has been added to a deblocked BLP substrate (see, FIG. 4) by biotin ligase.

FIG. 7 is a bar chart showing the results of the assay format described in FIG. 6. The first two columns represent samples, one reduced with Tris-(2-carboxyethyl)phosphine hydrochloride (TCEP) and the second not reduced with TCEP in the absence of biotin ligase. The third and fourth columns represent the same samples incubated with biotin ligase. Only the sample reduced with TCEP and incubated with biotin ligase produced a positive signal, indicating the sample became biotinylated.

FIG. 8 is a schematic representation of a reaction scheme for masking the s-amino group of lysine in a substrate for biotin ligase via 4-azidobenzyl carbamate formation.

FIG. 9 is a schematic representation of a DPC-mediated detection reaction based upon the ligation of two hemipeptides to produce a substrate for biotin ligase. Before ligation, the hemi-peptides are not recognized by biotin ligase. After ligation, the resulting product is recognized by the biotin ligase and biotin is added to the epsilon amino group of lysine in the peptide.

FIG. 10 is a schematic representation of exemplary hemi-peptides that can be ligated to form a substrate for biotin ligase. In this case, the N-terminal hemi-peptide contains a fluorescein molecule to enable the capture of the ligated peptide by an anti-fluorescein antibody in an ELISA assay. The two hemipeptides can be ligated in the presence of 1-Ethyl-3-[3-dimethylaminopropyl]carbodiimide hydrochloride (EDC).

FIG. 11 is a bar chart of the results of an assay system using the peptides described in FIG. 10. The positive control consisted of a full-length fluorescein-containing biotin ligase peptide. The peptides were used at two different concentrations, 2.5 and 0.25 mM, and EDC was added in two different concentrations, 1 mg/mL and 0.1 mg/mL. The amount of signal produced in the biotinylation reaction was highest in the presence of the higher concentrations of peptide and EDC. In the absence of EDC, the signal produced was equal to background (the same as omitting the hemi-peptide themselves).

FIG. 12 is a graph showing the results of ELISA assays showing the ability of monoclonal anti-T7 antibody to recognize only full-length T7 epitope peptide (denoted as full length) but not the two hemi-peptides (denoted as N-terminal and C-terminal, respectively).

FIG. 13 is a schematic representation of a reaction scheme for producing T7 hemi-peptide-oligonucleotide conjugates.

FIG. 14 illustrates the results of T7 peptide formation from T7 hemipeptide oligonucleotide conjugates (FIG. 14A) as characterized by gel electrophoresis (FIG. 14B).

FIG. 15 is a table showing exemplary peptide epitopes for which antibodies that bind to the peptide epitopes are commercially available.

FIG. 16 illustrates two examples of DPC reactions that can be used to facilitate peptide ligation via a thioester formation (FIG. 16A) or via a Staudinger Ligation (FIG. 16B).

FIG. 17 is a schematic representation of reaction schemes to produce thioester and phosphine peptides by solid phase peptide synthesis (SPPS).

FIG. 18 is a schematic representation of three approaches to reversibly deactivate a peptide that can activate the ribonuclease activity of ribonuclease S-protein. FIG. 18A shows peptide containing additional N- and C-terminal cysteines which can be used to circularize and inactivate the peptide through a disulfide bond. The disulfide bond can be broken by a sulfhydryl-reducing reagent. FIG. 18B shows a peptide where one or more lysines in the peptide are optionally diazotized, disrupting the recognition of the sequence by the S-protein. FIG. 18C shows two hemi-peptides of the full length S-peptide sequence that can be ligated to produce an active full-length product.

FIG. 19 is a schematic representation of an epitope creation reaction to detect a target sequence on a nucleic acid target. The two oligonucleotide-peptide conjugates anneal to adjacent or nearly adjacent complementary sequence on a target sequence. The localized high concentration of the peptides on the conjugations promote their rapid ligation upon annealing to the complementary sequences on the nucleic acid target.

FIG. 20 is a schematic representation of the test system demonstrating the DPC reaction of bisdiphenylphosphine reduction of diazidorhodamine (DAZR) described in FIG. 3 adapted to the detection of a specific target. In this case, two target binding components are directed against the A and B subunits of PDGF-AB. Each target binding component was separately zip-coded to hold an oligonucleotide conjugate containing a DAZR group and a bisdiphenylphosphine group, respectively. Simultaneous binding to the two target binding components leads to increased annealing of the reporter DNA sequences, increased proximity of the DAZR and bisdiphenylphosphine groups, and their rapid reaction to produce the fluorescent product rhodamine.

FIG. 21 is a graph showing the time course of the reaction of the assay format described in FIG. 20. The fluorescence of the rhodamine production was monitored over time. Negative controls were run omitting either the PDGF-AB target or the bisdiphenylphosphine oligonucleotide conjugates. A positive control included a large excess of free TCEP.

FIG. 22 is a schematic representation of an exemplary assay format where the signal is amplified with an anti-fluorescein antibody-horse radish peroxidase conjugate that preferentially binds to rhodamine over DAZR.

FIG. 23 is a graph showing the results of an ELISA assay to detect the reaction products from the reaction described in FIG. 22. A larger signal was obtained from the reactions which contained all the reactants compared to negative controls omitting the target molecule (PDGF-AB) or omitting bisdiphenylphosphine. The amount of signal produced in the presence of all the reactants was about equal the amount of signal produced in the positive control in which all DAZR was reduced with excess TCEP.

FIG. 24 is a schematic illustration of an exemplary DPC reaction scheme to produce a cyanine dye through an aldol type condensation reaction.

FIG. 25 is a schematic illustration of an exemplary DPC reaction scheme to produce p-Coumaric acid through aldol condensation.

FIG. 26 is a bar chart showing the detection of EGFR homodimers on A431 cells using two separate antibodies to EGFR each associated with a T7 hemipeptide, which when brought into proximity through DPC produce T7 peptide detectable by anti-T7 antibody.

FIG. 27 is a bar chart showing the detection of either EGFR homodimers or EGFR-ErbB2 heterodimers in A431 cells by flow cytometry using two separate antibodies to EGFR each associated with a T7 hemipeptide or an antibody to EGFR and an affibody to ErbB2 each associated with a T7 hemipeptide. When the antibody-hemipeptide complexes are brought into reactive proximity through DPC a T7 peptide is produced that is detectable by the incorporation of tyramide-Alexa 568 catalyzed by anti-T7 antibody-HRP conjugate.

FIG. 28 shows histograms demonstrating the flow cytometry distribution of KY01 cells treated with anti-T7 alone (FIG. 28A), a conjugate comprising an antibody to Bcr and a T7 hemipeptide reacted with anti-T7 antibody (FIG. 28B), and a conjugate comprising an antibody to Bcr and a T7 hemi-peptide with a conjugate comprising an antibody to Abl and a T7 hemipeptide reacted with an anti-T7 antibody (FIG. 28C).

FIG. 29A shows the flow cytometry distribution of KY01 cells treated with a conjugate comprising an antibody to Bcr and a T7 hemipeptide; a conjugate comprising an antibody to Abl and a T7 hemipeptide; or both a conjugate comprising an antibody to Bcr and a T7 hemipeptide and a conjugate comprising an antibody to Abl and a T7 hemipeptide. In each case, the conjugate-treated cells were reacted with an anti-T7 antibody, followed by detection with goat anti rabbit IgG F(ab)2-Alexa568 signal amplification. FIG. 29B depicts the flow cytometry distribution of purified bone marrow mononuclear cells treated with a conjugate comprising an antibody to Abl and a T7 hemipeptide; or both a conjugate comprising an antibody to Bcr and a T7 hemipeptide and a conjugate comprising an antibody to Abl and a T7 hemipeptide. In each case the conjugate-treated cells were reacted with an anti-T7 antibody, followed by detection with goat anti rabbit IgG F(ab)2-Alexa568 signal amplification.

FIG. 30 provides images of sections of human breast carcinoma tissue treated with none (FIG. 30D), one (FIG. 30C) or two separate antibodies to ErbB2 (FIG. 30A) each conjugated to a T7 hemi-peptide. For the two separate antibodies to ErbB2, each was associated with a T7 hemipeptide, which when brought into reaction proximity through DPC produced a T7 peptide. FIG. 30B shows a hematoxylin-eosin stained section. For each of FIGS. 30A, 30C and 30D, the cells were treated with anti-T7 antibody conjugated to HRP followed by detection with Tyramide-AlexaFluor568.

FIG. 31 shows a general reaction scheme for producing a native peptide bond using a first DNA-peptide conjugate having a C-terminal thioester and a second DNA-peptide conjugate having an N-terminal cysteine or cysteine analog.

FIG. 32 shows an exemplary reaction scheme for the synthesis of deprotected T7_p1_thioester.

FIG. 33 is a bar chart showing the effect of various pairs of mismatched or different sized reporter sequences, one linked to _p1-S(Et3MP) and the other to T7_p2_Cys, on the formation of a mutant T7 peptide by native chemical ligation.

FIG. 34 is a bar chart showing the detection of EGFR homodimers in an A431 cell line by DPC using native chemical ligation of T7_p1-S(Et3MP) and T7_p2_Cys and either a matched or mismatched pair or reporter oligonucleotides.

FIG. 35 is a bar chart showing the detection of a DNA sequence by DPC using either native chemical ligation (NCL) of T7_p1-S(Et3MP) and T7_p2-Cys or thioester exchange of T7_p1-S(Et3MP) and T7_p2-MA.

### DETAILED DESCRIPTION OF THE INVENTION

The invention permits the detection of a biological target (also referred to as a target molecule) in a sample. In the presence of the biological target, a DPC reaction produces a reaction product, for example, an intact epitope, enzyme substrate, enzyme activator, or ligand that can be detected, directly or indirectly, using an appropriate detection system. The detection system includes an amplification component that is capable of producing a plurality of detectable moieties per reaction product. The detectable moieties then can be detected visually or via an appropriate detector (for example, an optical detector, a fluorescence detector, a colorimeter, or an isotope detector). The appropriate detector will depend upon the detectable moiety generated in a given assay.

The invention provides methods, reagents, and kits for determining the presence and/or amount of a biological target in a sample, for example, a tissue or body fluid sample. In general, the assay systems include two probes, each of which comprises oligonucleotide conjugate that is capable of hybridizing to the other, a binding moiety for binding to the biological target and a precursor, for example, a product precursor or a masked product precursor. The components can be covalently or non-covalently associated with one another to produce a functional probe. When two such probes are combined with a sample, if the biological target is present, the binding moieties bind to the biological target, whereupon the oligonucleotides hybridize to one another to bring the product precursors or a masked product precursor and an unmasking group into reactive proximity to produce a reaction product (including unmasked reaction products). Thereafter, using the appropriate detection system, each of the reaction products can be used to generate a plurality of detectable moieties from each reaction product.

In certain embodiments, the product precursors (which lack an epitope) react with one another to produce a product that contains an epitope that can be detected by a detection component, for example, an antibody. Alternatively, the reaction product can be a ligand for a binding moiety, for example, a receptor, although the precursors are not bound by the detection component. Alternatively, the reaction product can be an activator and/or substrate of an enzyme, although the precursors do not activate and/or act as operative substrates for the enzyme. It is understood that the reaction product can be made by a synthetic scheme, a degradative scheme or by modification. The two precursor molecules can themselves be spontaneously reactive with one another, or may require the presence of other reagents or catalysts present in the solution to produce a reaction product.

The methods and compositions described herein can be used to determine the presence and, if desired, amount of a particular biological target in a sample of interest. The biological target can be, for example, a protein, peptide, nucleic acid, carbohydrate or protein. Exemplary proteins include, for example, a receptor, ligand, hormone, enzyme, or immunoglobulin. Exemplary targets include a protein complex, cell surface antigen, antibody, antigen, virus, bacteria, organic surface, membrane, or cellular organelles.

Under certain circumstances, the biological target can be a multimeric protein, for example, a homodimeric protein, a heterodimeric protein, or a fusion protein. The assays described herein can be used to determine the presence and/or amount of certain dimeric proteins and fusion proteins. In addition, the assays described herein can be used to determine the presence and/or amount of certain post-translationally modified proteins.

Exemplary multimeric proteins that can be detected and or quantified, include, for example, ErbB protein family homo- and heterodimers; VEGF receptor homo- and heterodimers; VEGF dimers; PDGF dimers; Tyrosine kinase receptor complexes; TNF/TNFR complexes; Cadherin complexes; Catenin complexes; IGFR complexes; Insulin receptor complexes; Receptor/receptor ligand complexes (e.g., EPO/EPO receptor); NF-kB/IkB complexes; T-cell antigen complexes; Integrin protein complexes; FKBP protein complexes; p53 protein complexes; Bcl family protein complexes; Myc/Max complexes; Cyclin protein complexes; Intracellular protein kinase complexes; Caspase protein complexes; Autoantibody-antigen complexes; and Secreted protein complexes (e.g., amyloid protein complexes).

Exemplary fusion proteins that can be detected and/or quantified, include, for example, Bcr-Abl; NPM-ALK; and certain ALK containing fusion proteins. Exemplary post-translational modifications that can be detected and/or quantified, include, for example, phosphorylated proteins (e.g., phosphorylated STAT proteins); glycosylated proteins; and farnesylated proteins (e.g., RAS).

It is understood that, with respect to probes described herein, the term “binding moiety with binding affinity to the biological target” is understood to mean that the binding moiety can bind directly or indirectly to the biological target. For example, the binding moiety can bind directly to the biological target, for example, where the binding moiety in the probe is an anti-ErbB antibody binds directly to the ErbB protein. However, it is also understood that the binding moiety can also bind indirectly to the biological target where, for example, the binding moiety of the probe can be, for example, a goat anti-mouse antibody, which during the practice of the invention described herein binds a mouse anti-ErbB antibody bound to the ErbB protein. It is understood that in the latter embodiment, the antibodies that actually bind the ErbB protein generally are different in some manner (e.g., are from different sources (e.g., where one antibody is derived from a mouse and the other antibody is derived from a rabbit) or have different structural features (e.g., antibodies with different Fc regions)).

Exemplary nucleic acids include a DNA (for example, genomic or complementary DNA (cDNA)) or portions thereof, or an RNA (for example, messenger RNA (mRNA), transfer RNA (tRNA), microRNA (miRNA), or ribosomal RNA (rRNA)) or portions thereof.

The targets can be detected using the methods and compositions described herein. However, it is understood that a particular assay format, depending upon certain considerations, for example, the biological target to be detected, the assay sensitivity desired, whether the assay is quantitative, semi-quantitative, or qualitative, may include one or more of the features, reagents and chemistries described herein. The following sections describe exemplary assay format, reagent considerations, and assay considerations.

I. Exemplary Assay Formats

The assay formats described herein generally involve the synthesis of reaction products from two or more product precursors, and/or the synthesis of reaction products from one or more masked (inactive) precursors.

An exemplary assay involving the synthesis of reaction products from two or more product precursors can be conducted as follows. The method comprises combining a sample to be tested with two probes. A first probe comprises (i) a first binding moiety with binding affinity to the biological target, (ii) a first oligonucleotide sequence associated (for example, covalently or non-covalently associated) with the first binding moiety, and (iii) a first product precursor associated (for example, covalently or non-covalently associated) with the first oligonucleotide sequence. A second probe comprises (i) a second binding moiety with binding affinity to the biological target, (ii) a second oligonucleotide sequence associated (for example, covalently or non-covalently associated) with the second binding moiety and capable of hybridizing to the first oligonucleotide sequence, and (iii) a second product precursor associated (for example, covalently or non-covalently associated) with the second oligonucleotide sequence.

The probes are combined with the sample under conditions to permit both the first and second binding moieties to bind to the biological target, if present in the sample. When the first and second binding moieties bind to the biological target, the first and second oligonucleotide sequences hybridize to one another to bring the first and second product precursors into reactive proximity with one another to produce a reaction product. The reaction product can be an intact epitope, an enzyme substrate, an enzyme activator or ligand.

The resulting reaction product, if present, then is exposed to a detection system capable of producing detectable moieties so that a single molecule of reaction product produces a plurality of detectable moieties. The detection component of the detection system interacts with the reaction product but not the product precursors and in association with the amplification component produces a plurality of the detectable moieties. The presence and/or amount of the detectable moieties is indicative of the presence and/or amount of the biological target in the sample.

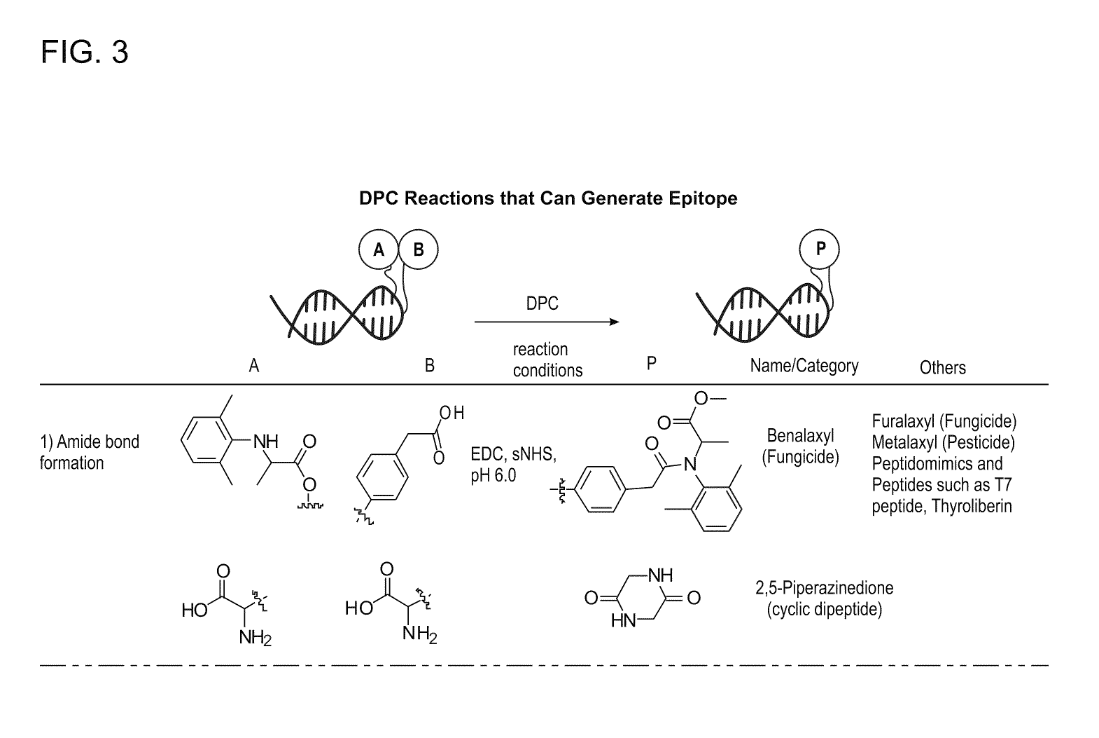

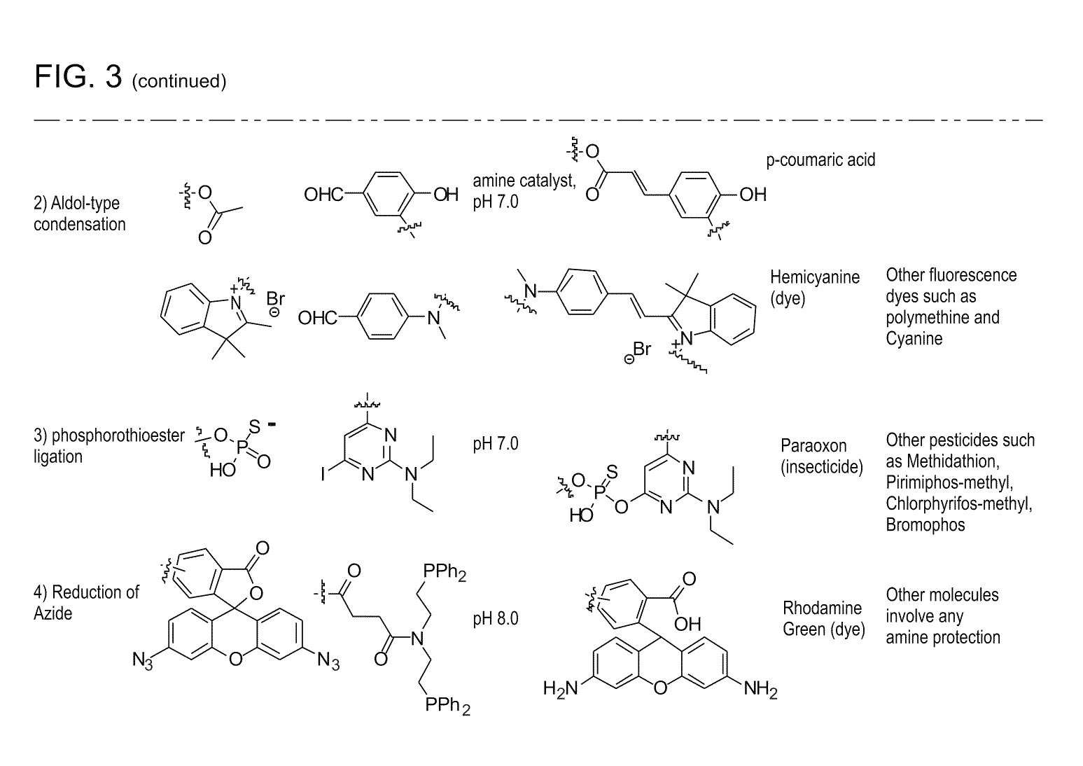

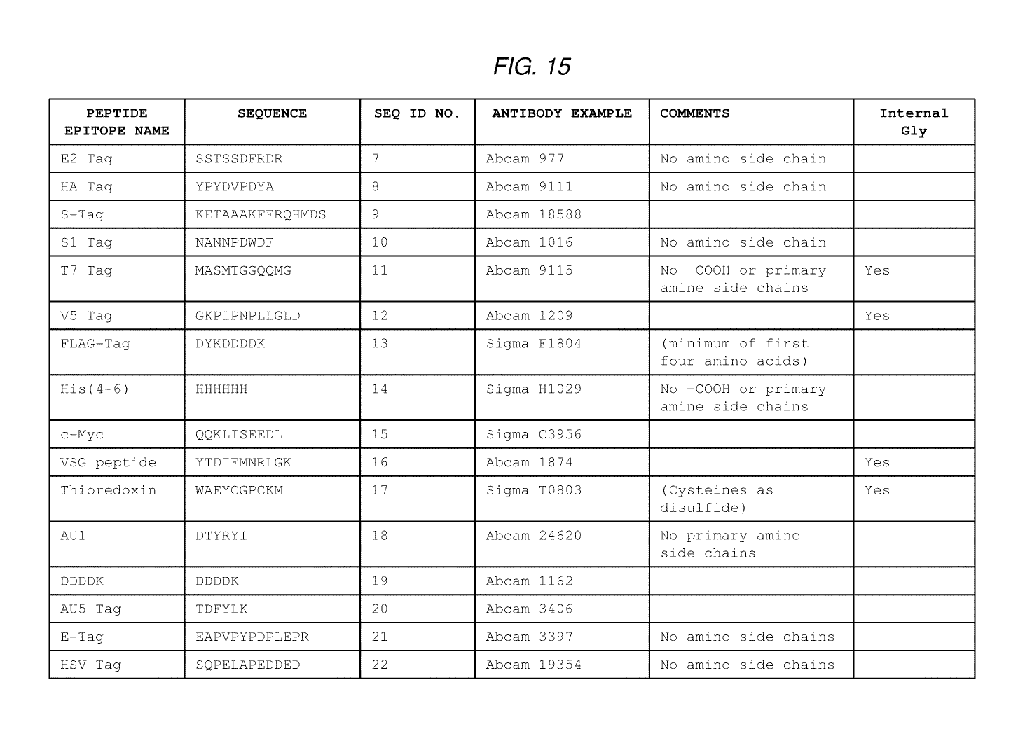

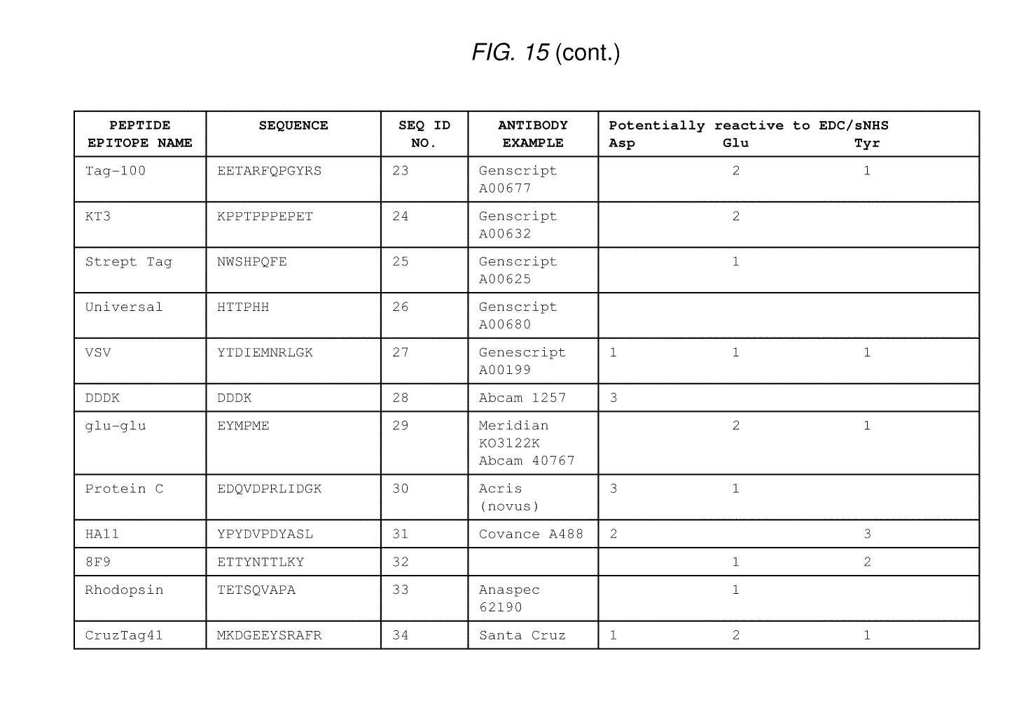

When the DPC reaction produces an intact epitope, it is understood that many known epitopes and their analogues can be generated through the foregoing reactions, which are also listed in FIG. 3. FIG. 15 also provides a list of epitopes, for which antibodies that bind to the epitopes are commercially available. In some cases, an effective DPC reaction can be designed to synthesize compounds and an antibody can be raised against such compounds. Similar reaction chemistries can be used to produce other reaction products, including, for example, enzyme substrates and enzyme activators.

Another exemplary assay involving the synthesis of reactive products from one or more inactive precursors can be conducted as follows. The method comprises combining the sample to be tested with two probes. The first probe comprises (i) a first binding moiety with binding affinity to the biological target, (ii) a first oligonucleotide sequence associated (for example, covalently or non-covalently associated) with the first binding moiety, and (iii) a first masked precursor associated (for example, covalently or non-covalently associated) with the first oligonucleotide sequence. The second probe comprises (i) a second binding moiety with binding affinity to the biological target, (ii) a second oligonucleotide sequence associated (for example, covalently or non-covalently associated) with the second binding moiety and capable of hybridizing the first oligonucleotide sequence, and (iii) an unmasking group associated (for example, covalently or non-covalently associated) with the second oligonucleotide sequence. The sample and probes are combined under conditions to permit the first and second binding moieties to bind to the biological target, if present in the sample. When the binding moieties bind to the biological target, the first and second oligonucleotide sequences hybridize to one another to bring the unmasking group into reactive proximity with the masked product precursor to produce a reaction product (an unmasked reaction product).

The resulting reaction product, if any, is exposed to a detection system capable of producing detectable moieties so that a single molecule of reaction product produces a plurality of detectable moieties. The presence and/or amount of the detectable moieties can then be used to determine the presence and/or amount of the biological target in the sample.

It is understood that the masked precursor can be a masked epitope, a masked enzyme substrate, a masked enzyme activator or a masked ligand. During the reaction, the masking group is removed to produce an unmasked product, for example, an unmasked epitope, unmasked enzyme substrate, unmasked enzyme activator or an unmasked ligand.

II. Reagents and Assay Conditions

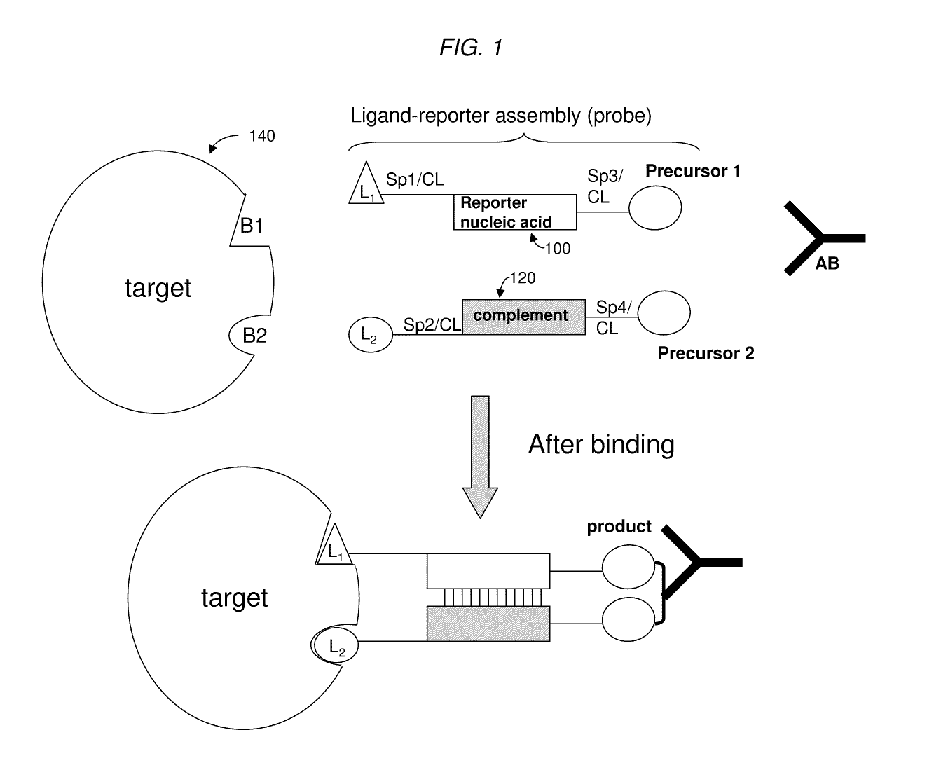

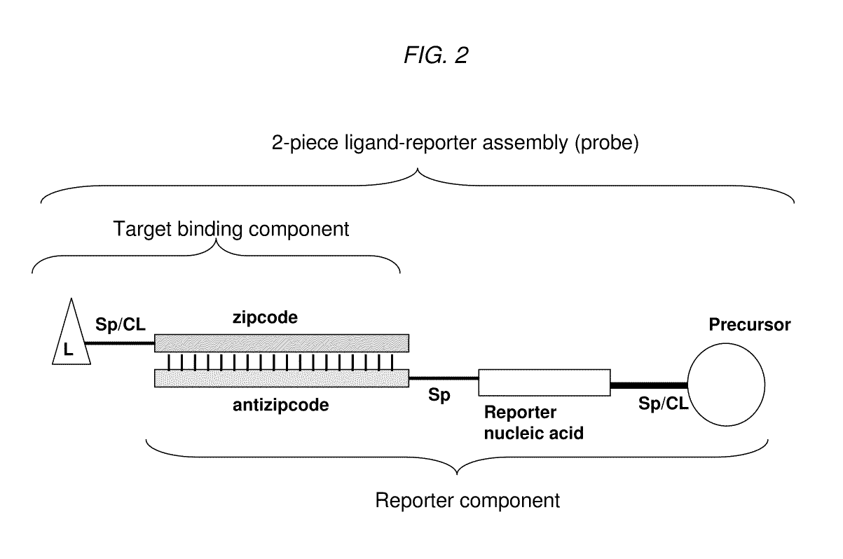

It is understood that a particular assay may use a number of the reagents and assay conditions disclosed herein. For example, the probes used herein, also referred to as ligand-reporter assemblies, can be a single molecule, for example, as shown in FIG. 1, or a plurality of molecules non-covalently associated with one another to produce a functional probe, as shown in FIG. 2.

As illustrated in FIG. 1, the reaction product is a peptide containing an intact epitope. However, the same principles can apply for the other reaction products described herein. In this format, the assay uses two ligand-reporter assemblies 100 and 120 wherein the precursors of the desired epitope (denoted precursor 1 and precursor 2) are each associated with an oligonucleotide (denoted reporter nucleic acid and complement, respectively). Furthermore, each ligand-reporter assembly contains a binding moiety (denoted L₁ and L₂, respectively) that binds to a corresponding binding site (denoted B₁ and B₂, respectively) on the target molecule 140. The binding moieties can include antibodies, adnectins, aptamers, or other molecules having binding affinity to the target. For the detection of nucleic acid targets, the binding moieties can be nucleotide sequences complementary to a target nucleic acid sequence or a portion thereof. Thus the target may also be a nucleic acid or any other molecule with two binding sites.

During the assay, the two binding moieties of the ligand-reporter complexes (L₁ and L₂) bind to each of the corresponding binding sites B₁ and B₂ on the target. Depending upon the biological target, it is understood that L₁ and L₂ can be the same or different. The “reporter DNA sequence” and “complement” represent nucleic acid, for example, DNA, sequences which are generally short, preferably 4-25 bases, more preferably 8-15 bases, in length and are complementary or substantially complementary to one another. The length of the nucleic acid, base composition and the degree of complementary sequence are selected such that the melting temperature (Tₘ) of the hybrid containing the two annealed nucleic acid sequences when bound to the target is typically somewhat above the ambient temperature (Tₘ) in the buffer system employed. The Tₘ of the hybrid containing the two annealed nucleic acid sequences in the absence of binding to the target is below ambient temperature.

In the assay format described in FIG. 1, the oligonucleotide sequences are covalently associated with the binding moieties and precursors via optional spacers (denoted Sp1, Sp2, Sp3, and Sp4) and/or cross-linkers (denoted CL). Varieties of heterobifunctional cross-linkers can be used to synthesize the ligand-receptor assembly. The most commonly used are: 1) amine-reactive and sulfhydryl-reactive cross-linkers, such as, succinimidyl-4-(N-maleimidomethyl)cyclohexane-1-carboxylate (SMCC); 2) aldehyde-reactive and sulfhydryl-reactive cross-linkers, such as, hydrazine/hydroxylamine and maleimide/iodoacetate functional group containing compounds; 3) aldehyde-reactive and amine-reactive cross-linkers, such as, hydrazine/hydroxylamine and succinimidyl functional group containing compounds (see, e.g., Hermanson, G. T. Bioconjugate Techniques, Academic Press 1996). In order to attach the precursor to a nucleic acid, it is usually functionalized, for example, with a carboxylic acid group, which then reacts with an amine-containing DNA. Other functional groups such as aldehyde and sulfhydryl groups can also be incorporated into the nucleic acid. In some cases, the precursor preferably is functionalized with, for example, hydrazone/hydroxylamine and maleimide group, respectively.

Sp 1 and Sp2 represent additional optional molecular spacers that are designed to add enough length to span the distance between binding sites on the target (for example, B₁ and B₂), such that the reporter nucleic acid and complement can anneal to each other while the binding moieties (L₁ and L₂) are bound to their targets. Sp1 and Sp2 can be DNA oligonucleotides which themselves include DNA monomers or oligomers, synthesized as a single piece of DNA with the reporters. However, the spacers may also contain other groups, such as, ethylene glycol oligomers, which may be incorporated using standard synthetic chemistries. Ethylene glycol spacers are often useful because they impart flexibility and hydrophilicity into the sequences. Sp3 and Sp4 are also optional spacers to prevent any steric hindrance interfering with the reactivity of the precursor molecules.

Precursor 1 and precursor 2 are two reactive (e.g., cross-reactive) chemical species, for example, small molecules that react to form a reaction product. Following simultaneous binding of binding moieties L₁ and L₂ to their corresponding binding sites on the target, the localized higher concentration of the ligand-reporter groups causes the reporter nucleic acid and the complement to anneal to one another (raising the Tₘ) bringing the precursors into reactive proximity with one another and at a higher concentration than in the bulk solution. The reactive precursors react to produce a product that can be detected by an antibody (denoted AB) that binds to the reaction product but not the two initial precursors. While FIG. 1 illustrates one type of synthetic reaction, the actual mechanism can differ provided the reaction creates a product that is recognized by an epitope-binding moiety. Alternatively, depending upon the assay format and the reaction product, the reaction product is recognized by, for example, an enzyme, a ligand or receptor. The reaction may occur simultaneously or may require one or more reactants, cofactors, or catalysts present in solution to facilitate the synthesis of the product.

Under certain circumstances, assembly of single molecules can be difficult because it requires a nucleic acid having two different functional groups at the 5′ and 3′ end, both of which should not react with one another but yet should still permit solid phase DNA synthesis and DNA cleavage conditions. If the 5′ functional group has to be incorporated into DNA in solution, a heterobifunctional cross-linker which does not cross-react to the 3′ functional group can be used.

In one approach, the probe can be synthesized as two or more separate pieces, which can then be assembled (for example, by a non-covalent association) to produce a functional ligand-reporter assembly. This can be performed by linking each binding moiety to a so-called zip-code sequence and separately linking the precursor molecule to a complementary anti-zip code sequence (see FIG. 2). For the two-piece ligand-reporter assembly, one ligand receptor assembly (denoted Target binding component) comprises the binding moiety (ligand) associated with an oligonucleotide having a zipcode DNA (denoted zipcode), an optional spacer (denoted Sp) and an optional crosslinking agent (CL). The other ligand receptor assembly (denoted Reporter component) comprises a product precursor (which, depending upon the chemistries employed could be a masked product precursor) associated with a reporter nucleic acid (for example, a DNA) that is associated with an anti-zipcode DNA sequence (denoted anti-zipcode), together with optional spacers. It is understood, however, that depending upon a particular assay, the order of these elements in the Reporter component may vary. For example, the order may include, for example, anti-zipcode-reporter nucleic acid-precursor or, for example, reporter nucleic acid-anti-zipcode-precursor, etc.

Each pair of zip-code and anti-zip code sequences should be designed to anneal with a relatively high Tₘ to each other, but to not anneal significantly to a second pair of zipcodes and anti-zip codes, nor to the reporter nucleic acid sequences. Each single self-assembled species non-covalently links the target binding moiety, reporter sequence, and precursor, but is stable in solution under typical reaction conditions.

The zipcode and anti-zipcode sequences typically are longer and form a more stable duplex than the reporter nucleic acid and its complement. This can be achieved by designing the zipcode and anti-zipcode sequences to be completely complementary and longer than the reporter nucleic acid. Typical zipcode and anti-zipcode sequences are 15-25 bases in length. The zipcodes typically are composed of DNA although they may be any type of nucleic acid (DNA, RNA, PNA, LNA). When an assay requires two ligand-reporter assemblies (see, FIG. 1) such an assay typically requires two Target binding components and two Reporter components, as shown in FIG. 2. The zipcodes and anti-zipcodes sequences are chosen such that each pair anneals only to each other, and not to other zipcodes or anti-zipcodes, nor to any reporter DNA sequences. Under these design conditions, the assay illustrated in FIG. 1 requires only that sufficient Reporter components are annealed to Target binding components. The 2-piece ligand-reporter assemblies (namely, probes) can also be pre-assembled, purified if desired, and added to the reaction mixture.

In certain embodiments, the assay is useful in the detection of a biological target having two binding sites, which may or may not be the same. In general, the assays described herein use two ligand-reporter assemblies, each of which includes (1) a binding moiety for the binding site of the target; (2) a reporter nucleic acid sequence, each one complementary or substantially complementary to the other reporter sequence of the pair; and (3) a product precursor, a masked product precursor or an unmasking group. If both binding moieties bind to their targets, then the localized higher concentration of the assemblies lead to a higher Tₘ, and formation of a nucleic acid duplex.

The binding moieties used in the probes can vary depending upon the target molecule to be identified. As discussed, the assays systems described herein can be used to detect a variety of biological targets in a sample. The assays are particularly useful in detecting protein multimers, for example, protein dimers, fusion proteins and glycosylated proteins. A variety of binding moieties, for example, antibodies, affibodies, adnectins, ligands, receptors, aptamers, and other binding molecules known in the art, can be used in the practice of the invention. Depending upon the target, the binding moieties used in each of the ligand-reporter assemblies can be the same or different.

For example, the invention is particularly useful in the detection of fusion proteins (e.g., BCR-ABL), receptor homodimers and heterodimers (e.g., homodimers and heterodimers of the ErbB receptor family, e.g., ErbB2 (HER2) homodimers, ErbB1 (EGFR) homodimers, EGFR/ErbB2 heterodimers, etc.), and multiple subunit-containing proteins (e.g., PDGF). For example, if the target is an EGFR/ErbB2 heterodimer, one binding moiety is selected to bind EGFR and other is selected to bind ErbB2.

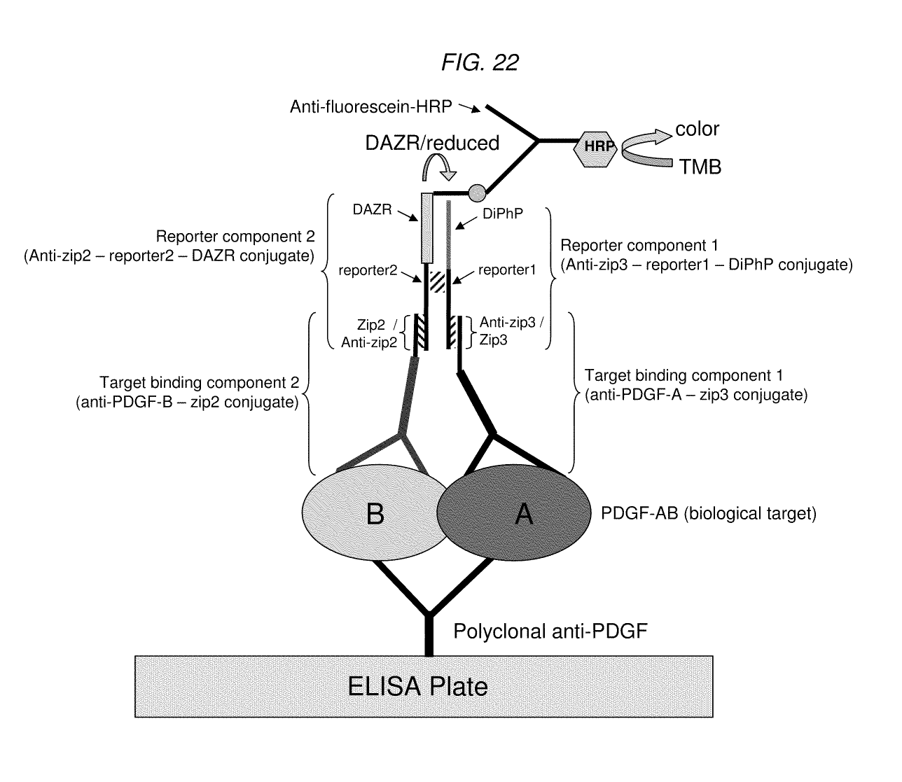

An exemplary assay format for the detection of a heterodimeric protein PDGF-AB is shown in FIG. 22. In this assay, PDGF-AB heterodimers are captured on the surface of a solid support, for example, the well of an ELISA plate. Thereafter, the PDGF molecules are exposed to two ligand-reporter assemblies, of the type shown in FIG. 2. A first target binding component (denoted target binding component 1) comprises an anti-PDGF-A antibody conjugated to a zip3 sequence and binds to subunit A of the heterodimer. A second target binding component (denoted target binding component 2) comprises an anti-PDGF-B antibody conjugated to a zip 2 sequence and binds to the subunit B of the heterodimer. A first reporter component (denoted reporter component 1) comprises an anti-zip3 sequence conjugated to diphenylphosphine, wherein the anti-zip3 sequence anneals to the zip 3 sequence of probe 1. A second reporter component (denoted reporter component 2) comprises an anti-zip 2 sequence conjugated to a rhodamine precursor. Once the reporter component 1 comes into reactive proximity with reporter component 2, as facilitated by hybridization of the reporter sequences (reporter 1 and reporter 2) in each of the reporter components, the rhodamine precursor is reduced to produce rhodamine Green. The presence of the rhodamine Green can be detected using a anti-fluorescein antibody-HRP conjugate, which binds to rhodamine Green but not to the rhodamine precursor. The HRP converts a substrate (TMB) into a colored detectable moiety.

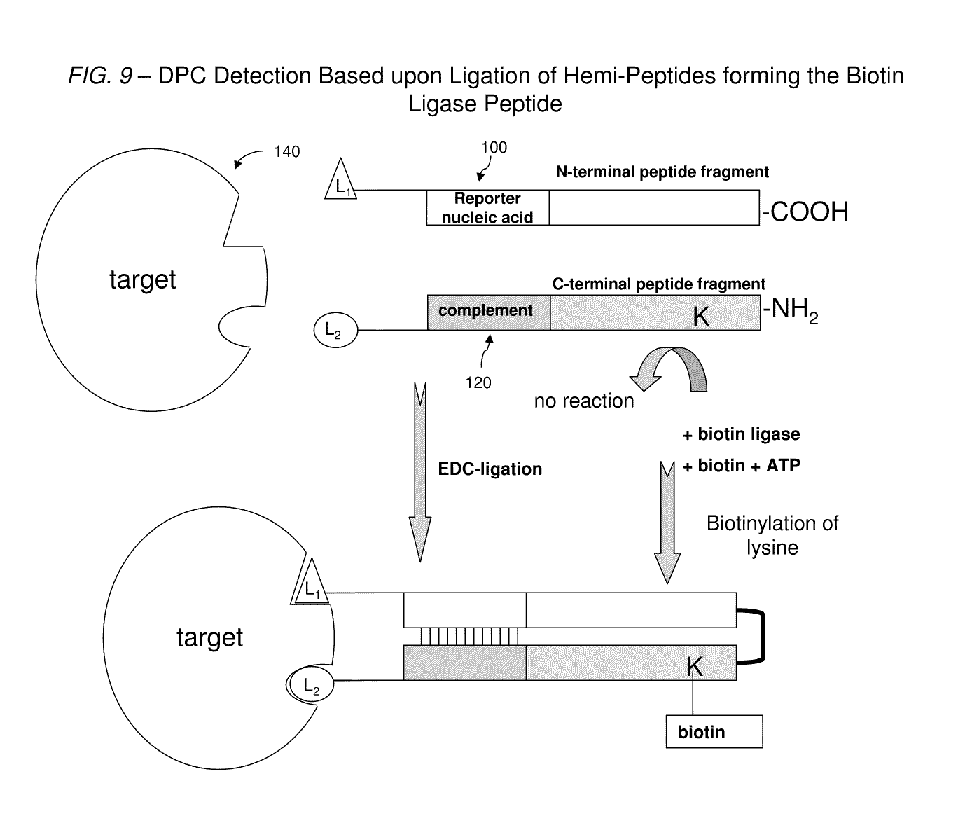

It is understood that the reaction product, rather than being an epitope, can also be, for example, an enzyme substrate or an enzyme activator. The synthesis of an exemplary enzyme substrate (a biotin ligase peptide) is described in Example 2. As shown in FIG. 9, an operative biotin ligase substrate is created by the DPC-mediated synthesis of the intact operative peptide from two inoperative hemi-peptides. Following synthesis, the biotin ligase added a biotin molecule to the peptide. The biotin can then be detected using a detection system containing an anti-biotin molecule (detection component) coupled to an enzyme (amplification component). The synthesis of an exemplary, operative enzyme activator, a S-13 peptide, that activates a mutant ribonuclease is described in Example 4.

In addition to assay formats where DPC facilitates the de novo synthesis of a reaction product, for example, an epitope, enzyme substrate or enzyme activator, it is understood that the reaction product can be produced from a masked product precursor containing one or more masking groups. During DPC, the masking groups are removed. For example, during DPC, a demasking agent is brought into reactive proximity with the masked product precursor containing the one or more masking groups. As a result, the masking groups are removed from the precursor containing the one or more masking groups. An exemplary assay format is described in Example 1. In Example 1, a modified BLP containing an azido-modified lysine is blocked against biotinylation with biotin ligase. The azido group is reduced by DPC to produce a primary amino group. The unmasked BLP can then act as a substrate for biotin ligase.

It is understood that any type of DPC-mediated chemical reaction that enables a formation of an operative epitope, enzyme substrate, enzyme activator or ligand can be used in the practice of the invention. However, in order to obtain a higher signal/noise ratio during the amplification process, the DPC reactions are preferably clean, fast and quantitative. Examples of useful DPC reactions include 1) amide bond formation through 1-Ethyl-3-[3-dimethylaminopropyl]carbodiimide hydrochloride (EDC) or native chemical ligation through thioester (NCL) for epitopes that comprise a cysteine amino acid residue or a cysteine analog (Dawson, P E et al., Science, 1994, 266, 776-779, 2) aldol condensation in the presence of amine catalyst, 3) phosphorothioester ligation (Xu, et al. J. Am. Chem. Soc. 2000, 122, 9040-9041), and 4) thioester/thioether peptide bond isostere ligation. Thioester replacement effectively replaces the nitrogen atom of the terminal amino acid of an epitope reactive fragment with a sulfur atom.

Without wishing to be bound by theory, it is contemplated that the following general guidelines can be used when determining which ligation method to use for the synthesis of peptide-based reaction products. In general, EDC/sNHS-mediated amide bond formation reactions, for example, as described in Example 2, are sensitive to steric hindrance by bulky amino acid side chains and this chemistry is most suitable when at least one of the amino acids involved in the ligation reaction is a glycine. EDC/sNHS may not be optimal for peptides containing Asp, Glu, Lys, or Tyr, or which contain more than two consecutive His in the C-terminus hemipeptide. In general, native chemical ligation, for example, as described in Example 12, requires the presence of an N-terminal cysteine residue as in the C-terminus hemipeptide. The Cys-containing peptide must be determined to retain its binding affinity for a particular binding moiety. In general, thioester bond formation requires that the N-terminal end of the C-terminus hemipeptide be Ala, Gly, His, Ile, Leu, Phe or Trp. Thioester bond formation should not be used if the peptide contains Cys due to interfering thiol exchange side-reactions.

It is understood that the production of a reaction product (for example, a product containing an intact epitope, a product that is an enzyme substrate, enzyme activator or ligand) may require additional reagents or reactants in solution to facilitate the reaction. If so, they can be provided in an excess concentration. Depending upon the assay format and whether the assay is quantitative or semi-quantitative rather than qualitative, the product precursors and unmasking groups may need to be rate-limiting. The reaction concentrations should preferably be provided such that the amount of reaction product (for example, epitope), and hence the amount of signal produced in the assay, is directly proportional to the amount of the biological target in the sample.

It is understood, however, that in the case of native chemical ligation, the reaction products (for example, peptides) are synthesized spontaneously. As a result, under certain circumstances the product precursors can react within one another even in the absence of binding to the biological target. This can be reduced or eliminated by using a number of approaches. Example 13 describes the benefits that can be achieved by lowering the Tₘ of the reporter oligonucleotide portions of the ligand-reporter assemblies (for example, in the range of from about 8° C. to about 25° C., more preferably from about 9° C. to about 20° C.) by introducing mismatches or by using sequences of different lengths. Each of these approaches has been found to improve the specificity of the detection systems.

The following examples contain important additional information, exemplification and guidance that can be adapted to the practice of this invention in its various embodiments and equivalents thereof. Practice of the invention will be more fully understood from these following examples, which are presented herein for illustrative purpose only, and should not be construed as limiting in any way.

### EXAMPLES

Example 1 describes a test system where a masked peptide substrate of biotin ligase was unmasked to become an operative substrate for the enzyme. Example 2 describes experiments where peptide fragments are ligated to one another to produce an operative enzyme substrate. Example 3 describes experiments where peptide fragments are ligated to one another to produce an intact epitope. Example 4 describes experiments relating to the synthesis of enzyme activator. Examples 5 and 6 describe experiments where the reaction product is a small molecule containing an intact epitope. Example 7 describes an assay format for detecting EGFR dimers. Example 8 describes an assay format for detecting EGFR and ErbB2 dimers. Example 9 describes an assay format for detecting Bcr-Abl fusion protein. Example 10 describes an assay format for detecting Bcr-Abl in CML-derived cell lines and bone marrow samples. Example 11 describes an assay format for detecting ErbB2 homodimers in breast cancer tissue. Example 12 describes the production of peptide containing an epitope by NCL. Example 13 describes approaches for increasing the specificity of assays where the reaction products are created by NCL. Example 14 describes exemplary hemi-peptides that can be produced during NCL. Example 15 describes an assay format for detecting EGFR homodimers using NCL. Example 16 describes additional reaction schemes for making peptides useful in NCL. Example 17 describes an assay format for detecting a DNA target through the formation of a T7 peptide containing an amide bond isostere.

Oligonucleotides described in the Examples were prepared using standard phosphoramidite chemistry (Glen Research, Sterling Va., USA) and purified by reversed-phase C18 chromatography. Oligonucleotides bearing 5′-amino groups were prepared using either 5′-Amino-Modifier 5 controlled pore glass (antizip oligo) or 5′-Amino-Modifier C6 controlled pore glass (zip oligo) and oligonucleotides bearing 3′-amino groups were prepared using 3′-Amino-Modifier C7 CPG (Glen Research, Sterling Va., USA). Sequences of various oligonucleotides used in the Examples are set forth in TABLE 1.

TABLE 1

| | | SEQ |
|---|---|---|
| | | ID |
| Oligo | Sequence (5′-3′) | NO. |
| Zip2 | TTGGTGCTCGAGTCCCCCCCCCCCCCCCCCCCCCC- | 62 |
| | NH₂ | |
| Zip3 | NH₂- | 63 |
| | CCCCCCCCCCCCCCCCCCCCGCTGCCATCGATGGT | |
| Zip5 | NH₂- | 64 |
| | CCCCCCCCCCCCCCCCCCGTGCCATCCATAGTCAG | |
| Zip2Zip5 | NH₂- | 65 |
| | TTGGTGCTCGAGTCCCCCCCGTGCCATCCATAGTCAG | |
| Antizip2 | GGACTCGAGCACCAATAC-X-TATAAATTCG-NH₂ | 66 |
| reporter | | |
| Antizip3- | NH₂-CGAATTTATA-X-CTGACCATCGATGGCAGC | 67 |
| reporter | | |
| Antizip5 | NH₂-CGAATTTATA-X-CTGACTATGGATGGCACG | 68 |
| reporter | | |

where X = Spacer Phosphoramidite 18 (Glen Research, Sterling VA, USA).

### Example 1

### Biotin Ligase Peptide—Unmasking of Biotinylation Site

Biotin ligase, in the presence of biotin and ATP, attaches a biotin molecule to a specific lysine residue present in a peptide sequence recognized by biotin ligase. The substrate for a biotin ligase, known as a biotin ligase peptide, which can be part of a larger sequence, can comprise the sequence LX₁X₂IX₃X₄X₅X₆KX₇X₈X₉X₁₀, wherein X₁=any amino acid; X₂=any amino acid except L, V, I, W, F, Y; X₃=F, L; X₄=E, D; X₅=A, G, S, T; X₆=Q, M; K=lysine; X₇=I, M; X₈=E, L, V, Y, I; X₉=W, Y, V, F, L, I; and X₁₀=preferably R, H but not D, E (Beckett, et al. (1999) A minimal Peptide Substrate in Biotin Holoenzyme Synthetase-catalyzed Biotinylation, Protein Science, 8, 921-929). Once the peptide is biotinylated, the presence of a biotin molecule can be detected using reporter molecules containing a binding moiety, such as avidin or streptavidin, which are commonly employed in the art.

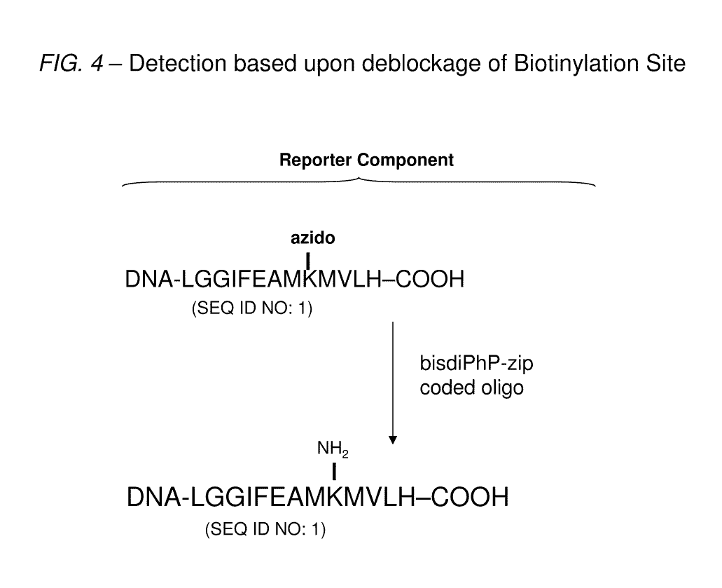

A modified BLP containing an azido-modified lysine is blocked against biotinylation with biotin ligase. The azido group can be reduced to a primary amino group in the presence of reducing reagents such as bis(diphenylphosphine) (FIG. 4). Alternatively, other agents such as lipoic acid and lipoamide can be used to reduce the azido group to a primary amino group.

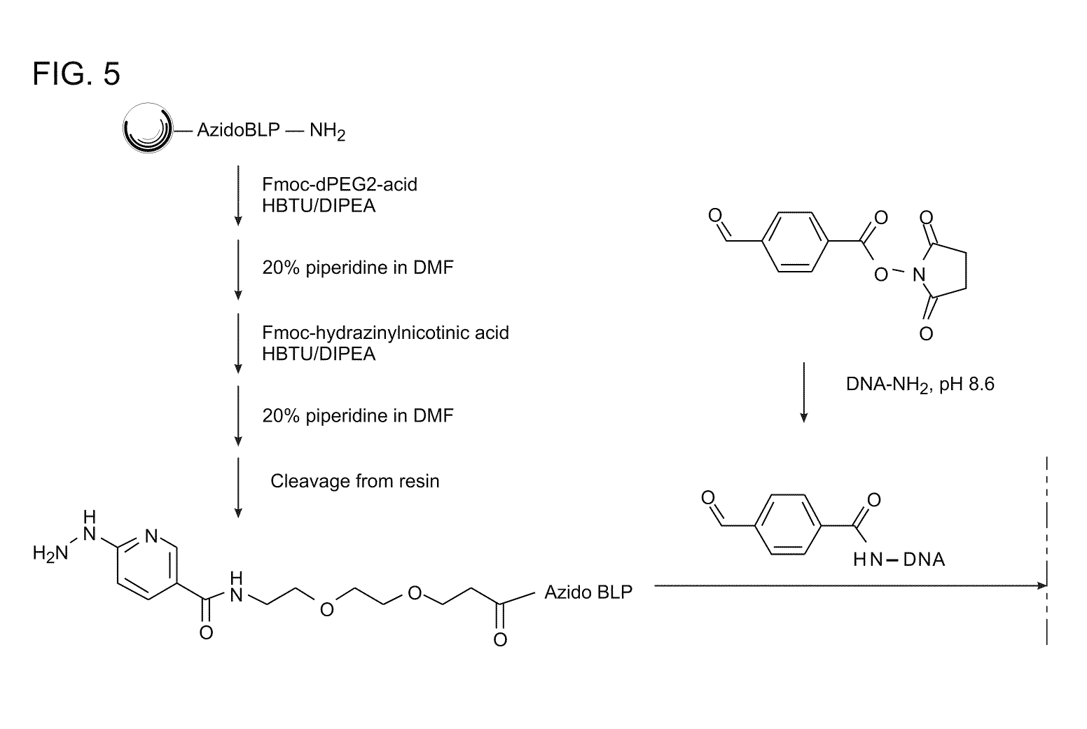

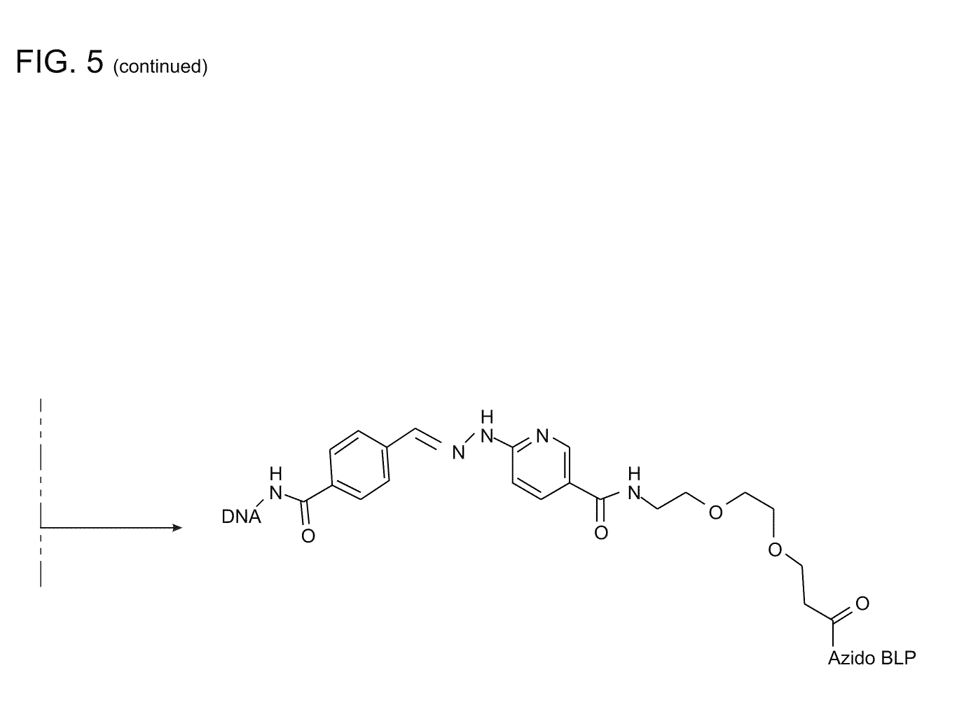

FIG. 4 represents the product precursor moiety of an anti-zipcode-reporter oligonucleotide-precursor conjugate (i.e., the product precursor moiety of a reporter component) of a two component ligand reporter assembly (probe) as previously described in FIG. 2. An oligonucleotide-peptide conjugate was synthesized in which a covalently associated precursor species contained the amino acid sequence LGGIFEAMKMVLH (SEQ ID NO: 1), in which the lysine residue (K) was modified with an azido group on the epsilon-amine of the lysine terminal side chain (see, FIG. 4). The epsilon-amine of Fmoc-Lys-OH was first converted to azido Fmoc-Lys-OH, and then assembled into the BLP through standard Fmoc solid-phase strategy. The azido BLP was linked to the oligonucleotide (denoted DNA) through hydrazone. Briefly, a hydrazine functional group was incorporated through coupling of Fmoc protected 6-hydrazinylnicotinic acid with azido BLP and an aldehyde functional group was added to the amine containing oligonucleotide as shown in FIG. 5. An additional polyethylene glycol (PEG) spacer was also incorporated in between azido BLP and DNA by coupling N-Fmoc-amino-dPEG2-acid to BLP to increase its flexibility (see, FIG. 5). On a second probe, Bisdiphenyl phosphine (bisdiPhp) was conjugated to the oligonucleotide through standard amide bond formation to produce the compound of Formula I.

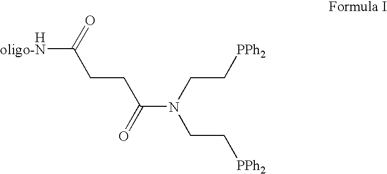

The first anti-zipcode-reporter oligonucleotide precursor conjugate contained an 18-base antizip oligonucleotide sequence, a ten base oligonucleotide reporter, and the azido modified peptide.

The first oligo-peptide conjugate was tested either not reduced or reduced in the presence of 4 mM TCEP, at 30° C. for 30 minutes in 50 mM sodium phosphate, pH 8. Following reduction, the conjugates were incubated in a 20 μL reaction mixture of biotin, ATP, and buffer (“BioMix A” and “BioMix B”) (Avidity, Inc., Aurora, Colo.) and 2 μg of biotin ligase (Avidity, Inc., Aurora, Colo.) for one hour at 30° C.

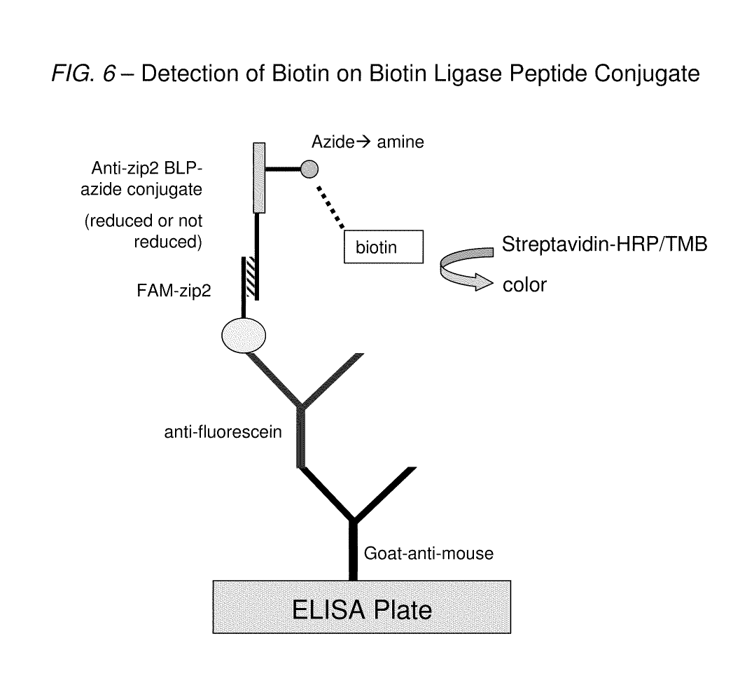

Once the azido group was reduced to a primary amine, the amino group could be recognized by biotin ligase and substituted with a biotin in the presence of ATP and biotin. The presence of a biotin on the lysine was detected by the assay format described in FIG. 6. Specifically, the conjugate was captured on an ELISA plate (solid support) containing an immobilized zipcode oligonucleotide, and detected with using a streptavidin-HRP conjugate (see, FIG. 6).

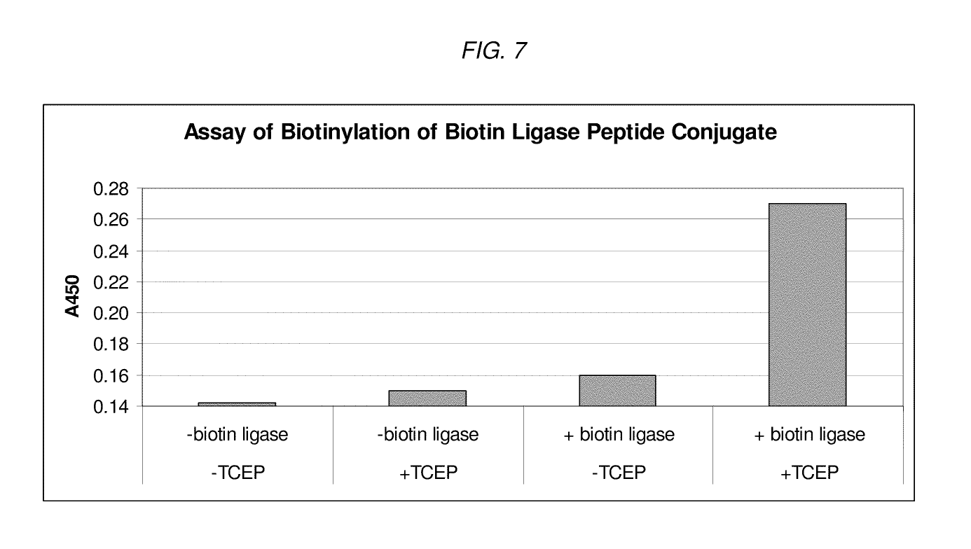

Briefly, ELISA plates were prepared by first coating ELISA plate wells substituted with goat anti-mouse antibody (Pierce, Rockford, Ill.) and then with 100 μL of 1:1000 diluted 1 mg/mL mouse monoclonal anti-fluorescein antibody (Roche Molecular systems, Pleasanton, Calif.) in PBS buffer. After washing, the wells were further incubated with 10 picomoles of the zip2 oligonucleotide labeled at its 5′ end with 6 carboxy-fluorescein to initiate identification of the anti-fluorescein antibody. Then, biotin-ligase oligonucleotide peptide conjugate (azido substituted), which was either not reduced or which was reduced with TCEP, was incubated and allowed to anneal to the immobilized zip2 capture oligonucleotide. The presence of biotin on the conjugate was detected by incubation with 1:4000 diluted 1 mg/mL streptavidin-HRP conjugate (Molecular Probes, Carlsbad, Calif.). The wells were washed and color developed in the presence of TMB substrate (KPL, Gaithersburg, Md.). The results are summarized in FIG. 7.

A positive response was observed only for conjugate which had been incubated with TCEP whereby the lysine residue had become unmasked. The unmasked lysine was then biotinylated by the biotin ligase.

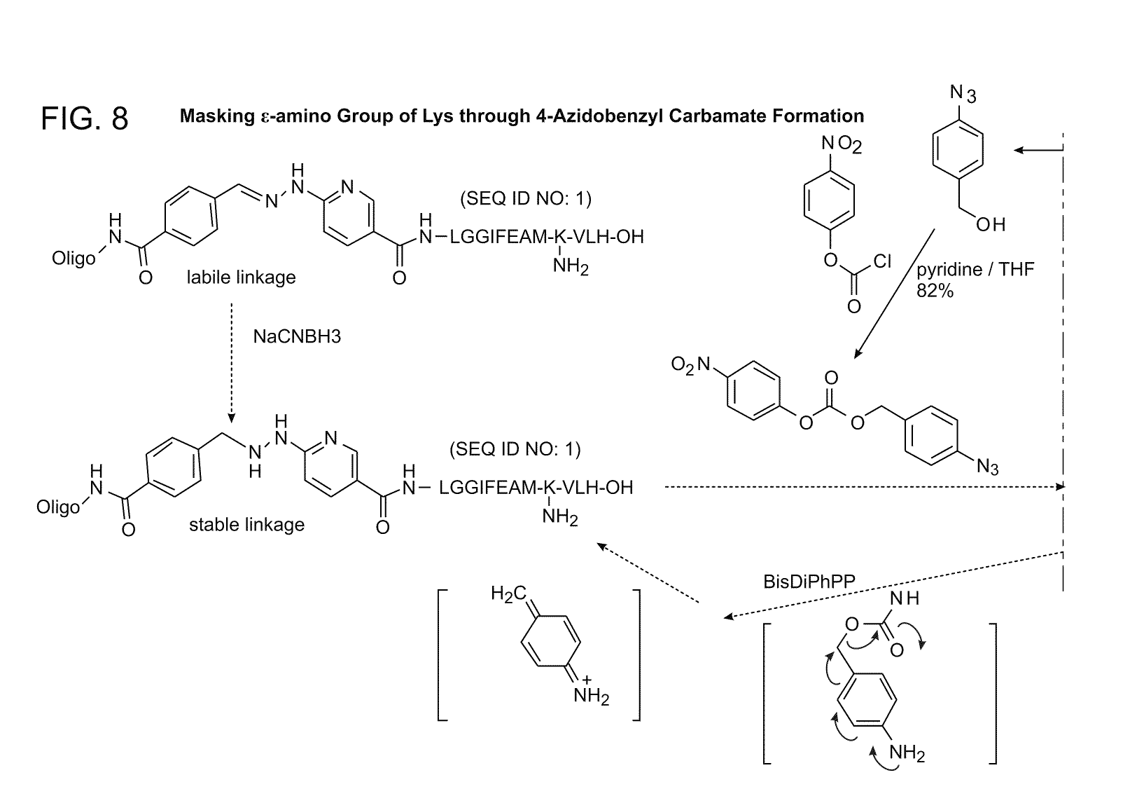

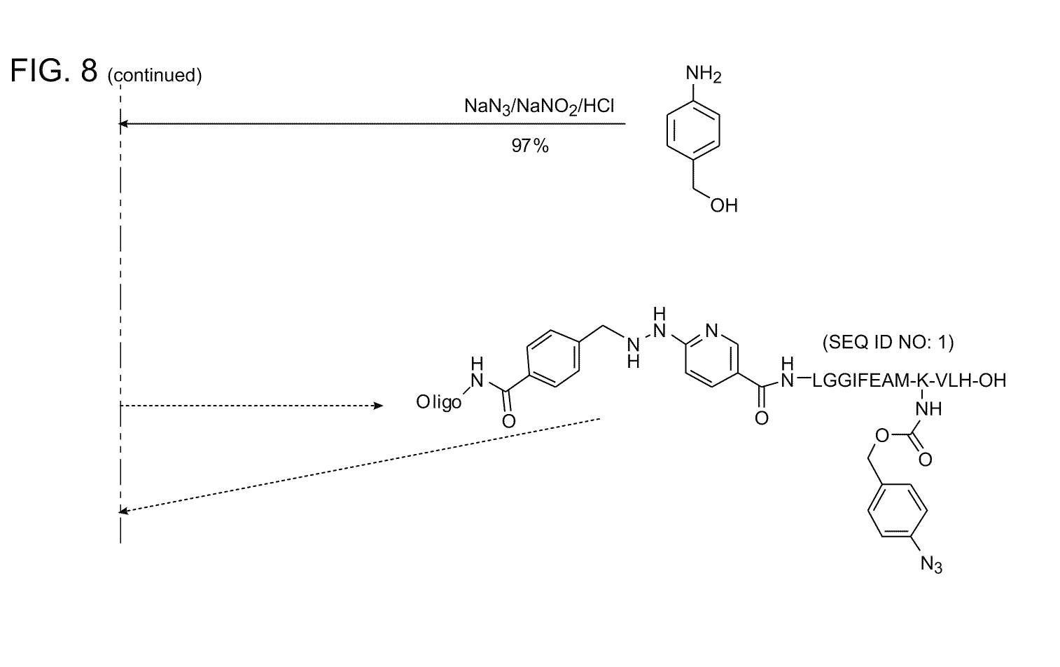

Another strategy of masking s-amino group of lysine uses 4-azidobenzyl-4-nitrophenyl carbonate to protect ε-amino with 4-azidobenzyloxycarbonyl (Mitchinson, et al. 1994, J. Chem. Soc. Chem. Commun. 2613-2614). Chemical reduction of the azide generates 4-aminobenzyl carbamate which undergoes spontaneous fragmentation via the intermediacy of the iminoquinone (Griffin, et al. 1996, J. Chem. Soc. Perkin Trans. 1, 1205-1211). The advantages of this strategy over the previous ones include easy reduction of aromatic azide over aliphatic azide, and the formation of a stable linkage through reductive amination of labile hydrazone linkage, since the protecting group could be incorporated after the synthesis of BLP-oligo. The general synthetic route to generate 4-azidobenzyl carbamate BLP-oligo is shown in FIG. 8. Briefly, diazotization-azidation of 4-aminobenzyl alcohol in aqueous hydrochloric acid afforded 4-azidobenzyl alcohol, which underwent further reaction with 4-nitrophenyl carbonochloridate to afford 4-azidobenzyl-4-nitrophenyl carbonate (80% yield over two steps). The 4-azidobenzyl-4-nitrophenyl carbonate was then reacted with NaCNBH₃ and reduced the BLP-oligo to produce 4-azidobenzyloxycarbonyl protected lys BLP-oligo.

Still another strategy for masking s-amino group of lysine or other amino groups is the protection with methionine and with analogous groups, such as, a thio(phenyl)ethyl carbamate. In this embodiment, one conjugate includes a precursor with an amino acid side chain amine group protected with methionine or an analogue thereof. The other conjugate contains a precursor with a reactive alkyl iodide group, such as, iodoacetamide. When the two precursors are brought into reactive proximity by DPC (e.g., by association of complementary reporter nucleic acids on respective conjugates), the methionine is cleaved leaving a free amine. Chemistries for adding methionine or thio(phenyl)ethyl carbamate to a free amine group and for adding iodoacetamide to a free amine are well known in the art.

A strategy for blocking a histidine side chain on one of the precursors can be accomplished using 2,6-dinitrophenyl (Shaltiel, S. et al., 1970 Biochemistry, 9: 5122-27). When a conjugate bearing this precursor is brought into reactive proximity with another conjugate bearing a precursor having a thiol group, the histidine is deprotected. This deprotection strategy is useful when utilizing either full-length StrepTag (WSHPQFEG—SEQ ID NO: 69) or truncated StrepTag (HPQFEG—SEQ ID NO. 70). For either precursor, protection of the histidine side chain blocks StrepTactin binding. Deprotection restores StrepTactin binding.

### Example 2

### Ligation of Peptide Fragments to Create an Enzyme Substrate

This example describes the detection of an operative enzyme substrate following its synthesis (ligation) from precursor fragments by DPC.

a) Biotin Ligase Peptide

The operability of this approach has been demonstrated using BLP. The minimum requirements for enzyme recognition of this peptide include a minimal length of 13 amino acids with specific amino acids specified at each site (see, the BLP sequence appearing in Example 1), including the requirement for a free primary amino group on the single lysine in the peptide. Fragments shorter than 13 residues usually are not recognized by biotin ligase.

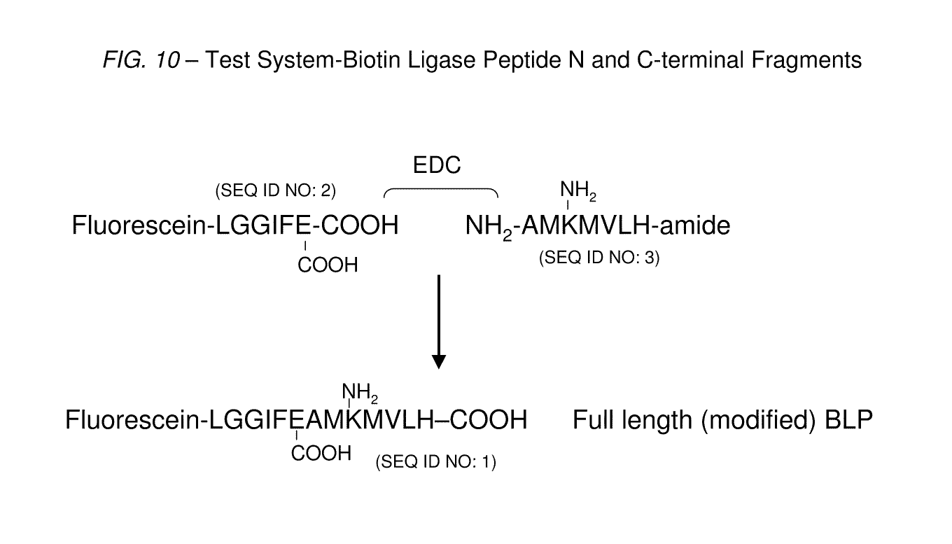

As shown in FIG. 9, a DPC-based detection assay can be based upon two ligand reporter assemblies each containing a partial length fragment (precursor) of the biotin ligase peptide. The carboxyl terminal of the N-terminal fragment and the amino terminal of a C-terminal fragment can be linked together in the presence of 1-ethyl-3-[3-dimethylaminopropyl]carbodiimide hydrochloride (EDC) as described in Hermanson, Greg T. in Bioconjugate Techniques, p. 170 (Academic Press, San Diego, 1996). The other two termini can be blocked by their synthesis as amides, or as shown in the test systems described in FIG. 10, with a fluorescein residue.

As illustrated in FIG. 9, the sample of interest is combined with two single molecule ligand reporter assemblies denoted 100 and 120. The ligand reporter assembly 100 contains a first binding moiety (denoted L₁) with binding affinity to target 140 linked to a first oligonucleotide sequence (denoted Reporter nucleic acid) which is linked to a first peptide fragment of a substrate for biotin ligase (denoted N-terminal peptide fragment). The second ligand reporter assembly 120 contains a second binding moiety with binding affinity to the target 140 (denoted L₂) linked to a second oligonucleotide sequence (denoted Complement) that is capable of hybridizing to the first oligonucleotide sequence and is linked to a second peptide fragment of a substrate for biotin ligase (denoted C-terminal peptide fragment). In the presence of target 140, both the first and second ligand reporter assemblies bind to the target whereupon the first oligonucleotide sequence (reporter DNA) and the second oligonucleotide sequence (complement) hybridize to one another to bring the N-terminal peptide fragment and the C-terminal peptide fragments into reactive proximity.

In the presence of EDC, the peptides become linked together to produce a full length peptide containing a free lysine side chain. Prior to ligation, the lysine present in the C-terminal fragment is not biotinylated with the biotin ligase in the presence of ATP and biotin. In contrast, after ligation of the peptide fragments, a peptide is generated where the free lysine becomes biotinylated by the biotin ligase in the presence of biotin and ATP.

FIG. 10 illustrates the amino acid sequence of two hemi-peptides that can be ligated in the presence of EDC to produce a substrate that can be biotinylated with biotin ligase. The N-terminal hemi-peptide (LGGIFE—SEQ ID NO: 2) has its N-terminal group blocked with fluorescein and the C-terminal hemi-peptide (AMKMVLH—SEQ ID NO: 3) has its C-terminus blocked with an amide group. The only other groups potentially reactive in the presence of EDC are the carboxyl side chain of glutamate (E) and the epsilon amino group of lysine (K). The reaction rate of the ligation depends upon the concentration of the peptide fragments, thus it is expected in a DPC-based assay that a localized concentration enhancement of the peptide fragments would yield a greatly increased reaction rate for the formation of a full-length biotin ligase peptide, which could then be biotinylated with biotin ligase.

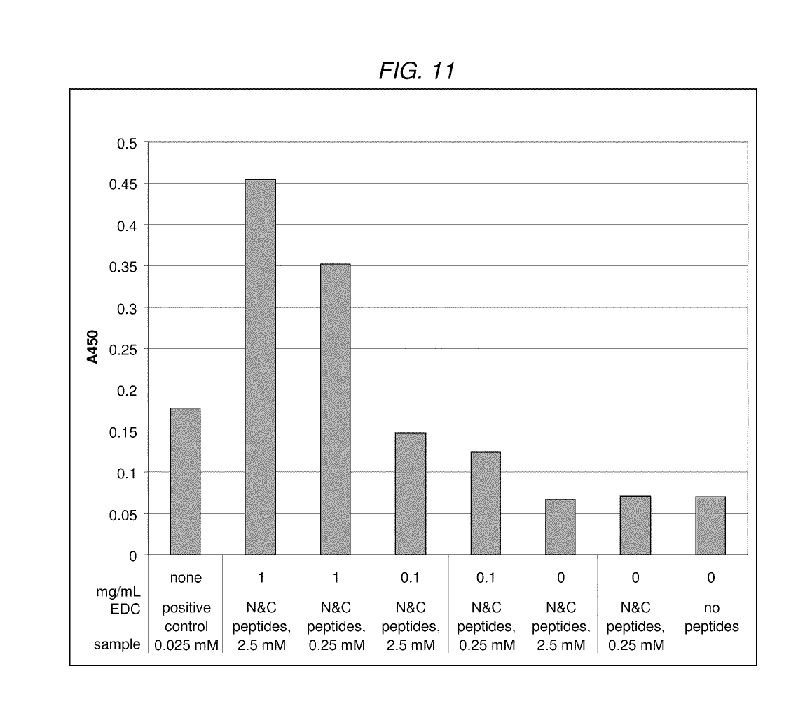

An ELISA assay for this ligation reaction was developed in which the ligation product (the intact ligated peptide with an N-terminal Fluorescein on the N-terminal peptide) was captured on a plate coated with an anti-fluorescein antibody. Fifty μL of peptide mixtures (2.5 mM) were incubated in the presence or absence of 1 mg/mL EDC in 0.1 M MES buffer, pH 6.5 for 1 hour at 25° C. Five μL of each mixture then was added to the wells of ELISA plate containing anti-fluorescein antibody. Then, 100 μL of biotinylation reaction mixture (as described in Example 1) was added to each well and incubated at 30° C. for 1 hour to biotinylate immobilized ligated peptide. The results are shown in FIG. 11.

It was found that the product of the EDC-catalyzed ligation of the two fragment peptides (each half the length of the full-length biotin ligase peptide) was biotinylated by biotin ligase while the unligated peptides in the absence of EDC were not biotinylated (see, FIG. 11). The reaction rate was faster in the presence of 1 mg/mL EDC than with 0.1 mg/mL EDC, and also faster in the presence of higher concentrations of the peptide (i.e., the reactions were more effective in the presence of 2.5 mM of peptides than with 0.25 mM peptides). The amount of signal (Absorbance at 450 nm) obtained from the ligation product was less at a fragment peptide input concentration of 0.025 mM as compared to 2.5 mM (at the same EDC concentration). The results indicate that the yield of full length ligation products was approximately 10%. This assay shows that the reaction rate increases at higher peptide concentrations and can occur in the presence of a catalyst which is free in solution.

Under certain circumstances, EDC chemistry is non-specific for ligation of carboxyl and amino groups and, therefore, under certain circumstances, may result in the random ligation of all possible pairs of primary amines and carboxyls in the peptides. In the case of biotin ligase peptides, not all the cross links would necessarily be located between the desired N- and C-terminals, but also could include cross links from the glutamine side chain carboxyl in the N-terminal hemi-peptide to the amino terminal of the critical lysine residue in the carboxyl terminal hemi-peptide. This nonspecificity of ligation could lead to a reduction in yield of the desired full length, unblocked peptide.

### Example 3

### Ligation of Peptide Fragments to Create an Epitope

This example describes the detection of an epitope created by DPC from peptide precursor.

i) ELISA Assay to Detect Full-Length T7 Epitope Peptide by Monoclonal Anti-T7 Antibody

The T7 epitope peptide can be created by the ligation of hemipeptides, both of which are required to reconstitute an operative T7 epitope, e.g., an epitope specifically bound by an anti-T7 antibody. The resulting full length peptide contains no highly reactive amino or carboxyl side chains. Accordingly, the T7 hemi-peptides can be ligated with EDC without undesirable cross reactions with other free amino or carboxyl side chains of other amino acids in the peptide.

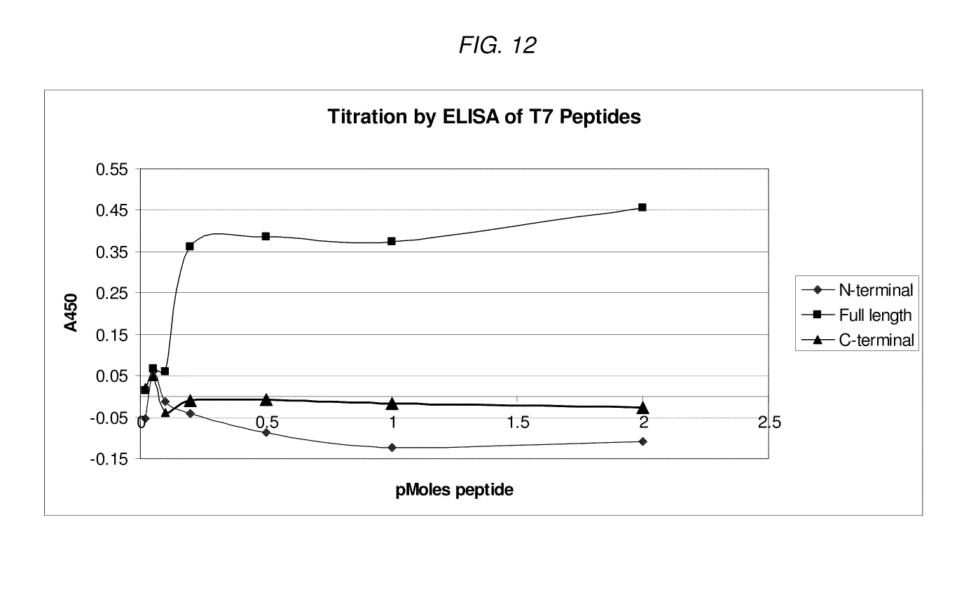

Two T7 hemi-peptides, an N-terminal hemipeptide and a C-terminal hemipeptide, were synthesized. The N-terminal amine of the N-terminal hemi-peptide and the C-terminal carboxyl of the C-terminal hemi-peptide were both synthesized as amides, leaving only one free amine and carboxyl free to react. The N-terminal peptide was conjugated with fluorescein to yield fluorescein-MASMT (SEQ ID NO: 50) and the C-terminal peptide was GGQQMG (SEQ ID NO: 71). Accordingly, the full length peptide was fluorescein-MASMTGGQQMG (SEQ ID NO: 4). The peptides were ligated using EDC as described in Examples 1 and 2. The hemi-peptides, or the ligated, fluorescein-labeled full length peptide, were exposed to anti-fluorescein antibody immobilized in the wells of an ELISA plate, and then were detected with an anti-T7 antibody conjugated with horse radish peroxidase (Novagen, Gibbstown, N.J.). The results are summarized in FIG. 12, where the monoclonal anti-T7 antibody only recognized the full length epitope. The precursor hemi-peptides produced no detectable signal response.

ii) Synthesis of T7 Peptide From Hemi-Peptides Via EDC Mediated DPC

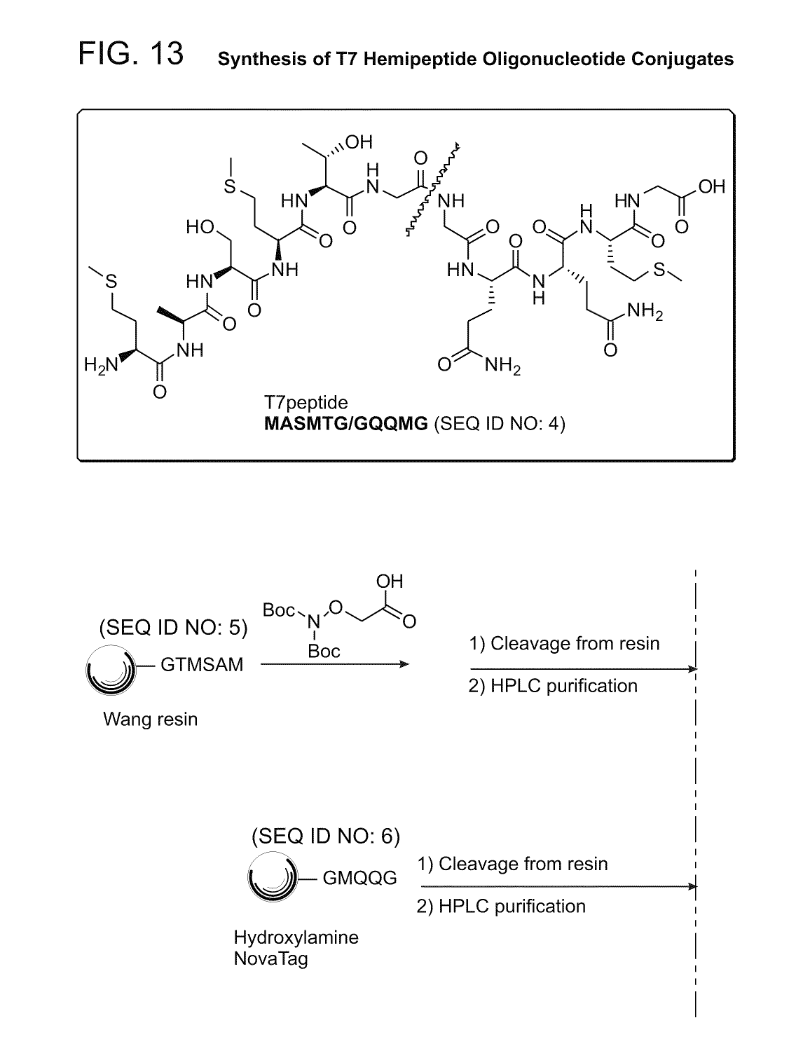

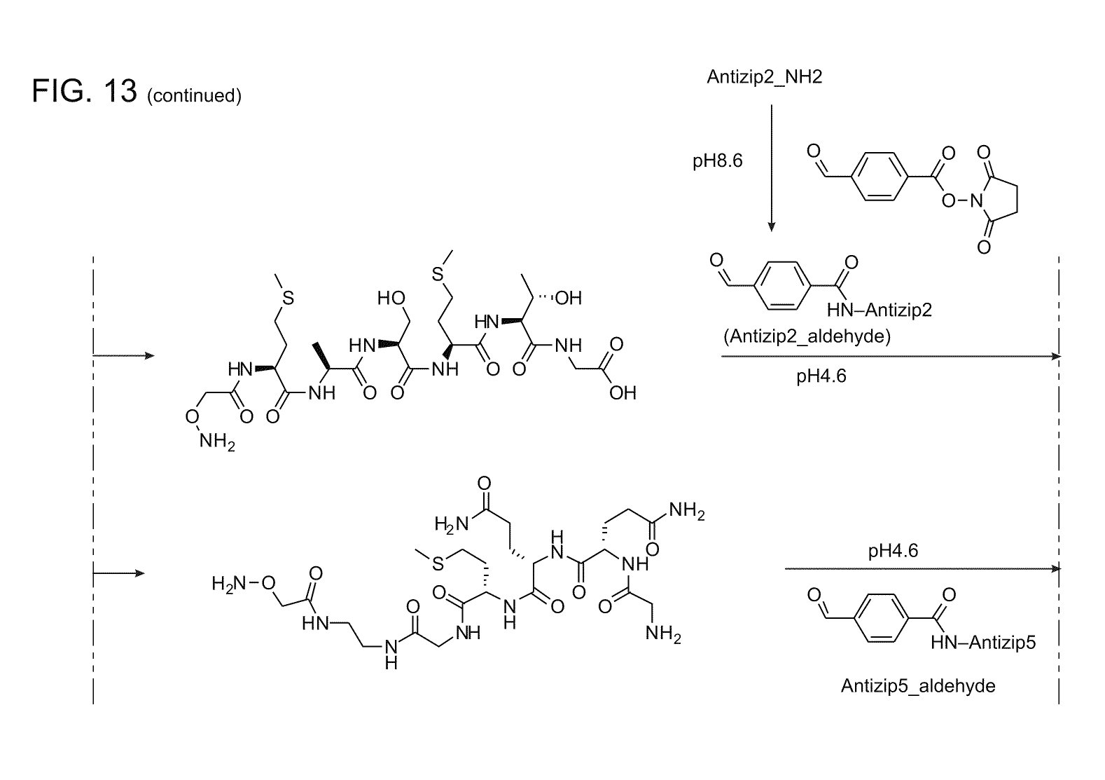

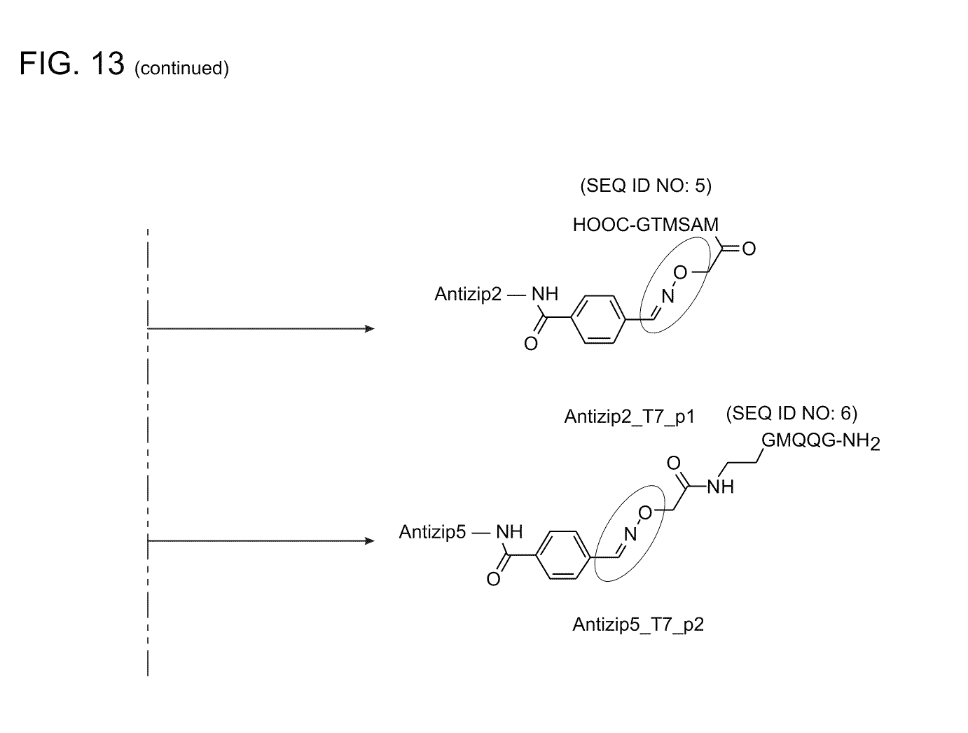

Two T7 hemipeptides were synthesized and conjugated to oligonucleotides via oxime formation as shown in FIG. 13. The N-terminal hemi-peptide MASMTG (SEQ ID NO: 5) (T7_p1) included a free carboxylic acid group at its C-terminal and a hydroxylamine group at its N-terminal for oligonucleotide conjugation. The C-terminal hemi-peptide GQQMG (SEQ ID NO: 6) (T7_p2) included a free amine group at its N-terminal and a hydroxylamine group at its C-terminal for oligonucleotide conjugation.

Both hemi-peptides were synthesized by standard Fmoc solid phase strategy. The side-chain functional groups of Ser and Thr were protected with the tert-butyl group, and the free amide side chain of Gln was protected with a Trityl group. Peptide coupling was performed by standard o-benzotriazole-N,N,N′,N′-tetramethyl-uronium-hexafluoro-phosphate (HBTU) coupling and the Fmoc group was deprotected using 20% piperidine in DMF.

After synthesis, the peptides were cleaved from the resin using a buffer of TFA (80% in water v/v), water (5% v/v), thioanisole (5% v/v), ethanedithiol (2.5% v/v) and phenol (7.5% w/v). Crude peptide then was purified by preparative HPLC (XTerra Prep MS C18 OBD, 5 μm, 19×100 mm column, Waters), in which peptide was eluted using a TFA gradient (holding 0% B from 0 to 2 minutes, then gradient 0-30% B over 15 minutes followed by 30-95% for 2 minutes; buffer A: 0.1% TFA in water; buffer B: 0.1% TFA in acetonitrile) at 20 mL/min. To introduce a hydroxylamine functional group into the N-terminal hemipeptide N-terminus-MASMTG-C-terminus (SEQ ID NO: 5) (T7_p1), bis-Boc-amino oxyacetic acid was coupled to the terminal methane group at the end of solid phase peptide synthesis (SPPS) and the free carboxylic acid was obtained as the result of cleavage of Wang resin. About 3 mg of pure product T7_p2 was isolated from 50 mg of crude material (6% recovery, calculated mass for C₂₄H₄₃N₇O₁₁S₂: 669.25, obtained: M+H 670.2531 Da). The hydroxylamine group was conveniently introduced into C-terminal hemipeptide N-terminus-GQQMG-C-terminus (SEQ ID NO: 6) (T7_p2) using hydroxylamine NovaTag resin (NovaBiochem, Gibbstown, N.J.). About 10.2 mg of final product T7_p2 was obtained from 42 mg of crude product (24% recovery, calculated mass for the molecule C₂₃H₄₂N₁₀O₉S: 634.29, and obtained mass: M+H 635.3265).

The hemipeptides (T7_p1 and T7_p2) then were conjugated to a pair of complementary DNAs containing aldehyde functional groups (referred to as Antizip2_aldehyde and Antizip5_aldehyde in FIG. 13 with sequences as described in Table 1). Briefly, 2 nmole of DNA_aldehyde was combined with 20 nmole of hemipeptide in 20 μL of 200 mM sodium phosphate buffer, pH 4.6 and mixed at 37° C. HPLC analysis confirmed that after 1 hour, no starting DNA_aldehyde remained in the reaction mixture. The product was then purified using an analytic C18 column (Waters XTerra C18, 3.5 μm, 4.6×50 mm) in TEAA gradient buffer (Buffer A: 0.1% TEAA, pH 7.0; Buffer B: acetonitrile; Gradient 5-30% B over 15 minutes, then 30-80% over 5 minutes, final gradient 80-100% over 5 minutes at 1 mL/min) and analyzed by LC-MS. Mass data: Antizip2_aldehyde: calculated monoisotopic mass for C₃₀₀H₃₈₉N₁₀₉O₁₇₇P₂₉ [M-7]⁻⁷: 1320.0947, obtained: 1320.1364; Antizip5_aldehyde: calculated monoisotopic mass for C₃₀₁H₃₈₉N₁₀₇O₁₈₀P₂₉S₂ [M-7]⁻⁷: 1324.6631, obtained: 1324.7086; Antizip2_T7_p1: calculated average mass for C₃₂₄H₄₃₀N₁₁₆O₁₈₇P₂₉S₂ [M-7]⁻⁷: 1413.8617, obtained: 1413.5623; Antizip5_T7_p2: calculated average mass for C₃₂₄H₄₂₉N₁₁₇O₁₈₈P₂₉S [M-7]⁻⁷: 1413.4237, obtained: 1413.1477.

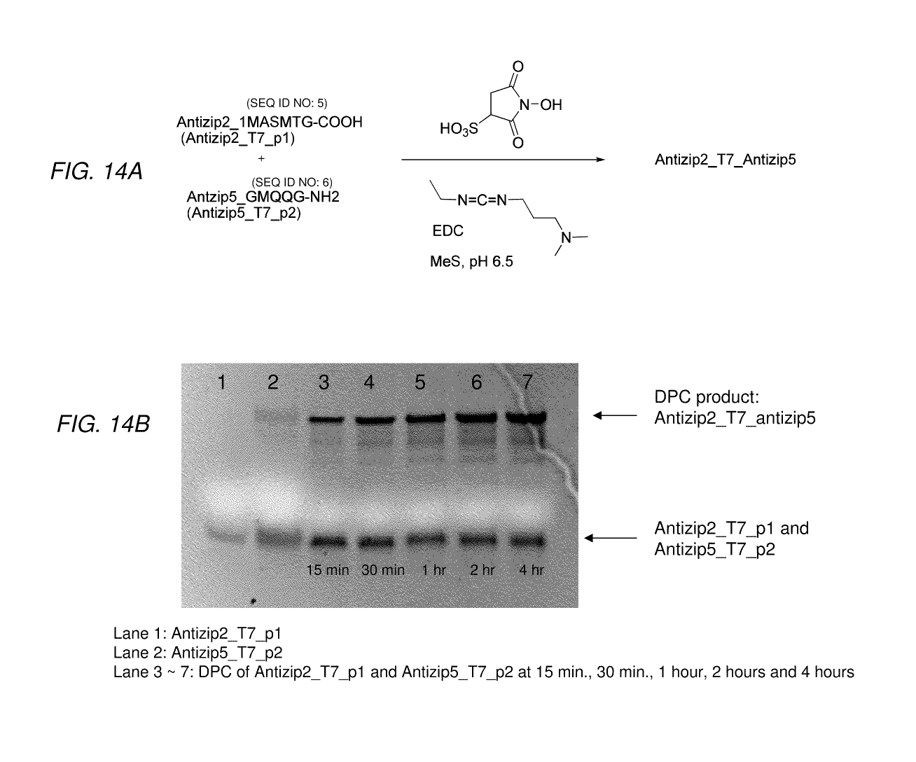

The DPC reaction of antizip2_T7_p1 and antizip5_T7_p2 was performed in the presence of EDC and sulfo-NHS (N-hydroxysulfosuccinimide sodium salt) as shown in FIG. 14A. The addition of sulfo-NHS was not necessary but resulted in higher yield. However, EDC reacted with carboxyl group to form an active ester (O-acylisourea) leaving group, which could be hydrolyzed before it encountered the target amine in aqueous solutions. The hydroxyl group on Sulfo-NHS can react with the EDC active-ester complex and increase the stability of the active intermediate, which ultimately reacts with the attacking amine. The reaction was performed in a total reaction volume of 800 μL and contained 0.2 μM of each of Antizip2_T7_p1 and Antizip5_T7_p2, as well as MES (0.1 M, pH 6.0), NaCl (150 mM), EDC (20 mM) and sNHS (15 mM) and was conducted at room temperature. Aliquots of 50 μL were taken out after different time intervals and desalted by chromatography on a Bio-Rad P-6 size exclusion column. After drying under vacuum, the sample was redissolved in 14 μL of TBE-urea sample buffer and heated at 45° C. for 3 minutes, cooled down on ice, then analyzed by gel electrophoresis (15% TBE-urea gel, 300 volt, 25 minutes). The gel was visualized by ethidium bromide stain and the results are shown in FIG. 14B.

FIG. 14B shows that the T7 peptide was formed within 15 minutes (about 30 to 40% product formation). The product band intensified with increasing reaction times while the starting material were diminished. HPLC analysis of the DPC reaction mixture after 4 hours showed the product peak (same method as purification of antizip2_T7_p1 but the column was run at 35° C.), which was collected and confirmed by LC-MS.

(iii) Other Peptide Epitopes

The protocol described above can be used to create epitope containing peptides. A large number of other peptide sequences are known for which specific antibodies have been identified (see, e.g., The Epitope Binding Peptide Database, which can be found on the world wide web at imtech.res/in/raghava/mhchem/index/html). FIG. 15 provides a list of a small subset of peptide epitopes for which there are also commercially available antibodies. These peptide epitopes usually are employed as affinity tags for the isolation of genetically engineered proteins because these epitopes appear otherwise infrequently in other proteins. In many cases, the minimum length of the peptides and affinities of antibodies for sequence variations of these epitopes for antibody binding has been identified.

FIG. 15 also identifies which of the sequences have no amine or carboxyl side-chain containing peptides and which contain an internal glycine. The lack of amino or carboxyl side chains is desirable for selection of hemi-peptides which ligate with high specificity using EDC coupling. The presence of an internal glycine can provide a convenient break point in the peptide sequence for the hemi-peptides using several of the available chemistries known in the art.

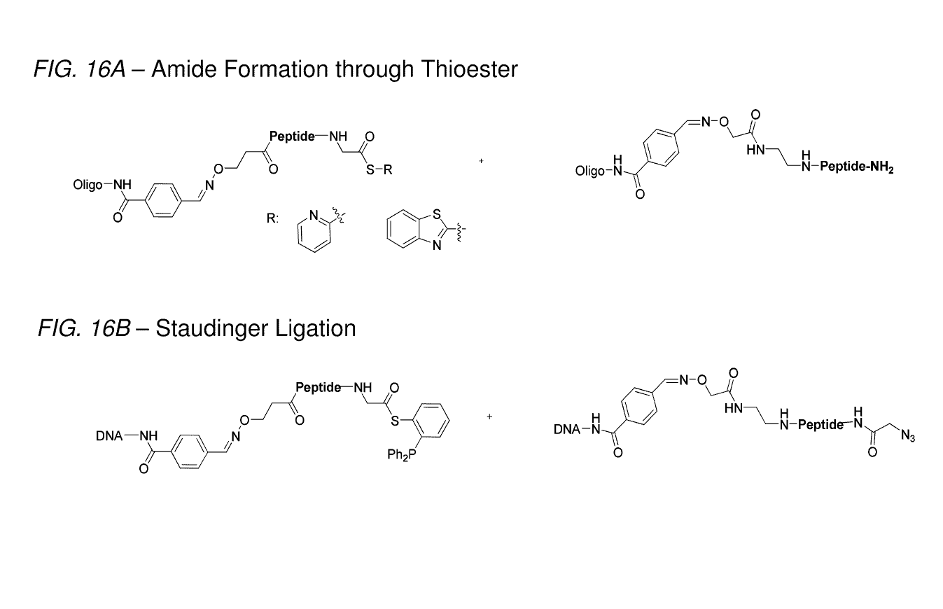

Two examples of suitable reaction schemes for selective ligation of peptides that containing potentially reactive side chains groups are shown in FIG. 16. FIG. 16A shows amide formation through a thioester moiety. A thioester generally reacts specifically with N-terminal Cys (trans-thioesterification) yielding a thioester-linked intermediate which undergoes spontaneous, rapid intramolecular reaction to form an amide bond (Dawson, et al. 1994, Science 266, 776). It has been reported that thioesters with certain leaving groups, such as, pyrimidyl and benzothiazole can react with amino group directly without the addition of a metal catalyst (Benaglia, et al., 2005, A. Tetrahedron, 61, 12100-12106). The addition of metal catalysts may be detrimental for DNA. FIG. 16B shows a Staudinger ligation reaction between a thioester and an azide (Nilsson, et al., 2000, Org. Lett. 2, 1939-1941).

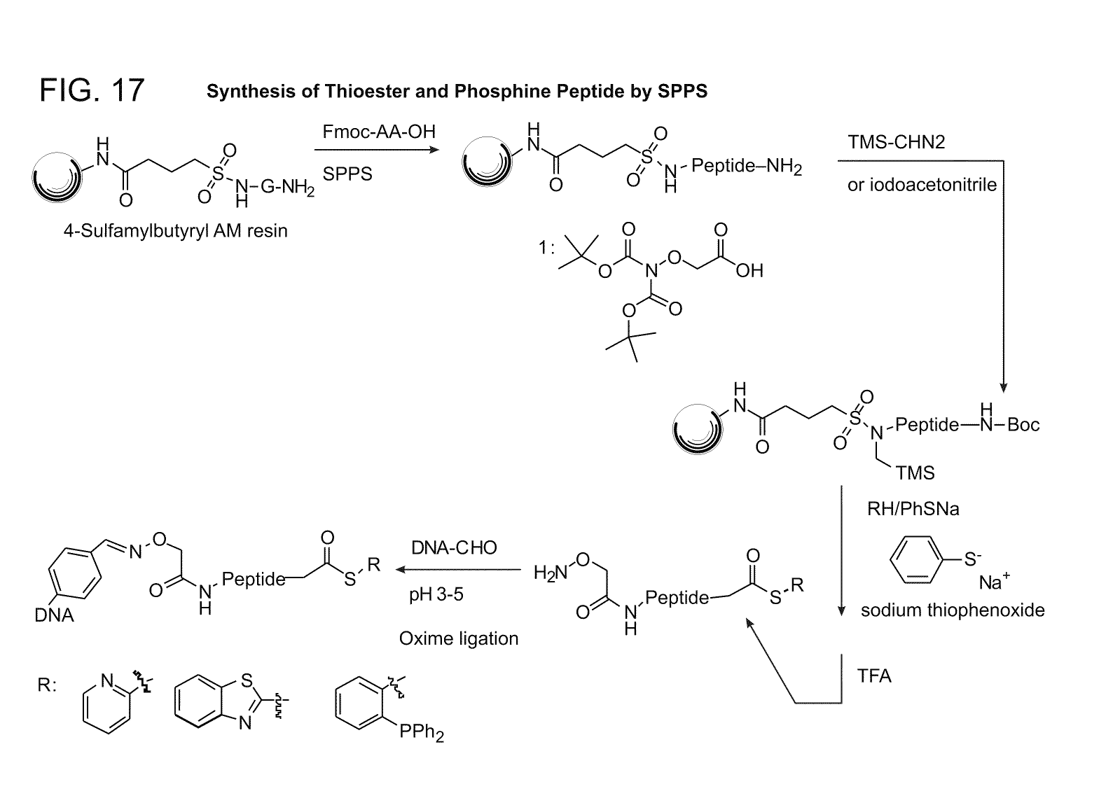

Similar reaction schemes can be used to conjugate peptides with oligonucleotides to construct assemblies of the present invention. FIG. 17 shows a reaction scheme for synthesizing a thioester and phosphine peptide DNA conjugates by solid phase peptide synthesis (SPPS).

### Example 4

### Creation of Enzyme Substrates

It is contemplated that strategies similar to those described in Examples 2 and 3 can be employed to create peptide sequences other than peptides containing epitopes. For example, some enzymes require the presence of a particular peptide sequence for activity. One example is the substrate for the ribonuclease S-protein enzyme, a deletion mutant of ribonuclease that depends upon the presence of a 15 amino acid long peptide for activity (S-15 peptide) (Levit, et al., 1976, “Ribonuclease S-Peptide” J. Biol. Chem. 251 (5) 1333-1339). Accordingly, substrate peptides that are shorter than 15 amino acids have much lower or no ability to activate the ribonuclease activity of S-protein. The same reactions as proposed for ligation of peptide epitopes described above, can be used to ligate inactive fragments of S-15 peptide to form an operative S-15 peptide capable of reconstituting ribonuclease activity. The 5-15 peptide, therefore, is an operative enzyme activator of the ribonuclease S-protein enzyme. In this way, ribonucleases can be used as a signal reporting enzyme. Ribonucleases have rapid turnover rate and can trigger fluorescence generation using quenched fluorescent substrates (Kelemen, et al., 1999 “Hypersensitive Substrate for Ribonucleases” Nucleic Acids Research 27, No. 18, 3696-3701).

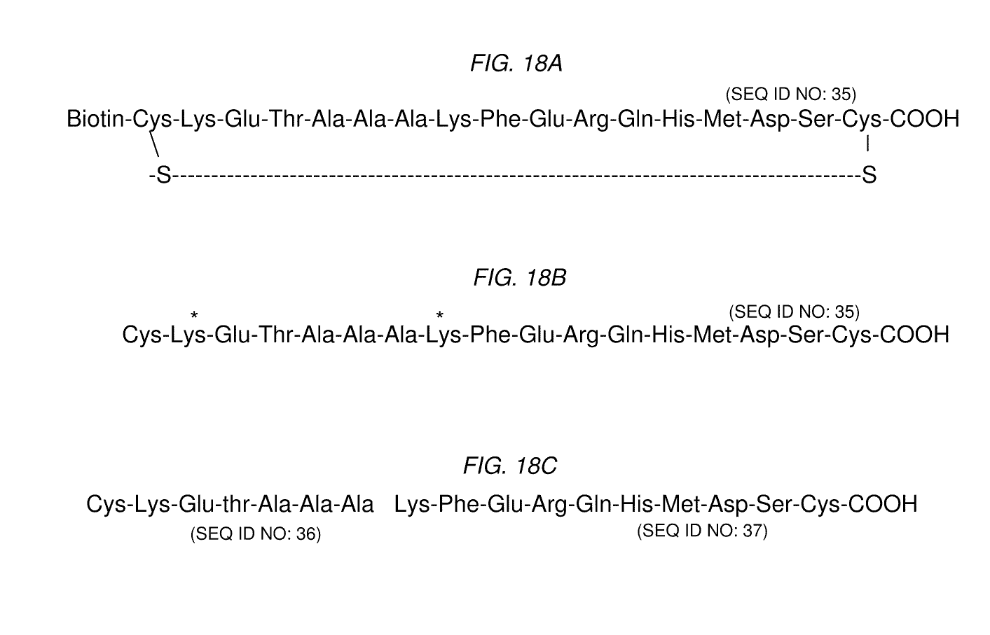

FIG. 18 shows three ways to create an inactive S-15 peptide, which can then be activated by DPC. For example, as shown in FIG. 18A, the S-15 peptide can be inactivated by circularizing the peptide via a disulfide bridge between N- and C-terminal cysteines added to the S-15 sequence. The disulfide bridge can then be reduced with a reducing agent, for example, a diphenylphosphine-oligonucleotide conjugate, to produce an active S-15 peptide. As shown in FIG. 18B, one or both of the internal lysines (denoted by asterisks) of the peptide are converted into diazo groups to inactivate the peptide. The thia azido group can also be reduced, for example, with a diphenylphosphine-oligonucleotide conjugate using the same strategy as employed for the reduction of the diazo group with the biotin ligase peptide. As shown in FIG. 18C, the S-15 is split into two hemipeptides—one with a C-terminal thioester and the other with an N-terminal cysteine. The resulting hemipeptides, neither of which activate ribonuclease S-protein alone can be ligated in the methods of this invention via native chemical ligation.

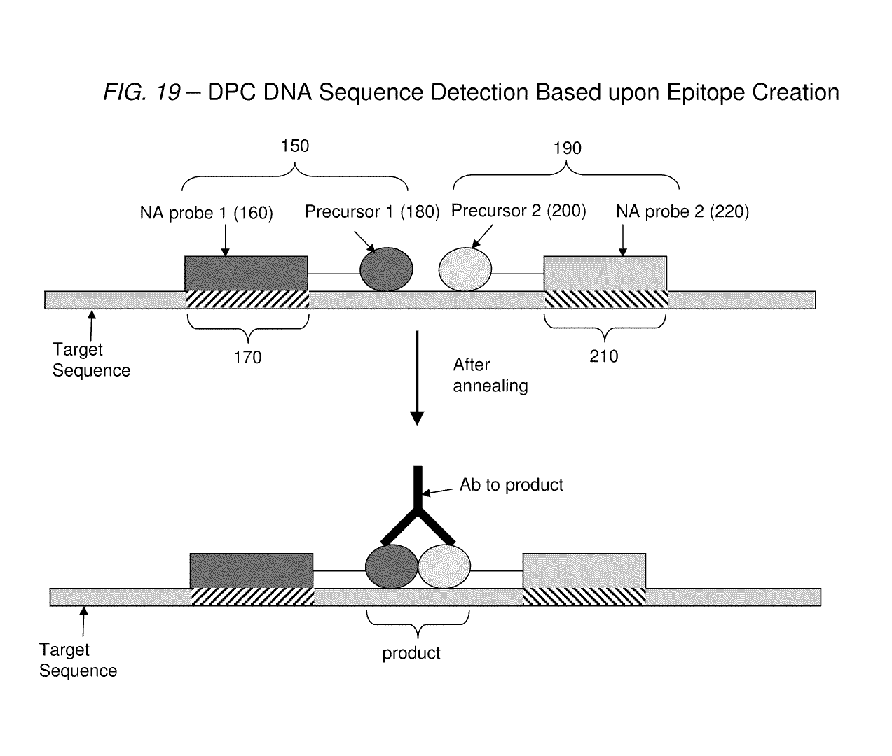

As has been discussed, ligand reporter assemblies containing a binding moiety, such as an antibody, and a reporter group, can be used for non-nucleic acid biological targets. However, using similar chemistries as described above, the conjugates also can be used for the detection of nucleic acid targets as shown in FIG. 19. In this case, the reactants are similar to the protein-based detection reagents (see, FIG. 1) except that the “nucleic acid reporters” and “complements” are not self-complementary, but rather are probes that anneal to adjacent (or nearly adjacent) complementary sequences in a target DNA.

As shown in FIG. 19, a first probe (ligand reporter assembly) 150 contains a nucleic acid sequence 160 (denoted NA probe 1) that anneals to a complementary or substantially complementary sequence 170 in the target sequence conjugated to a peptide precursor 180 (denoted Precursor 1). A second probe (ligand reporter assembly) 190 contains a nucleic acid sequence 220 (denoted NA probe 2) that anneals to a complementary or substantially complementary sequence 210 in the target sequence conjugated to a peptide precursor 200 (denoted Precursor 2). The sequences in regions 170 and 210 in the target sequence can be the same or different. Once the probes hybridize to the target regions they bring the two precursors into reactive proximity to produce a peptide product defining an epitope. The product can then be detected using a binding moiety, such as an antibody (denoted AB), that specifically binds to the product.

### Example 5

### Creation of Dyes as Epitopes

Simple dyes, such as fluorescein, can also serve as epitopes. For example, a DPC assay can involve the reduction of the non-fluorescent molecule diazidorhodamine (DAZR) with diphenylphosphine to produce a fluorescent dye rhodamine Green (see, FIG. 3). The rhodamine Green can be detected directly since it binds to an anti-fluorescein antibody. For higher sensitivity, the rhodamine Green dye can be detected by an anti-fluorescein antibody conjugated to a reporter enzyme.

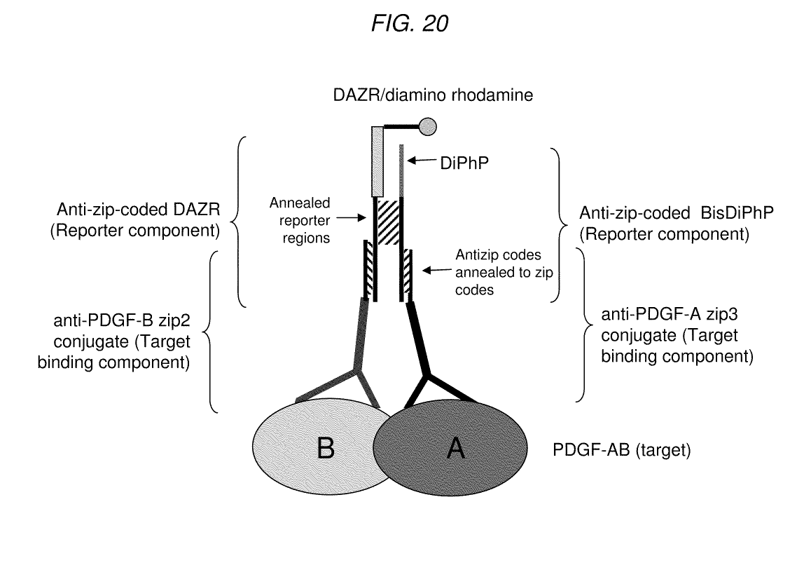

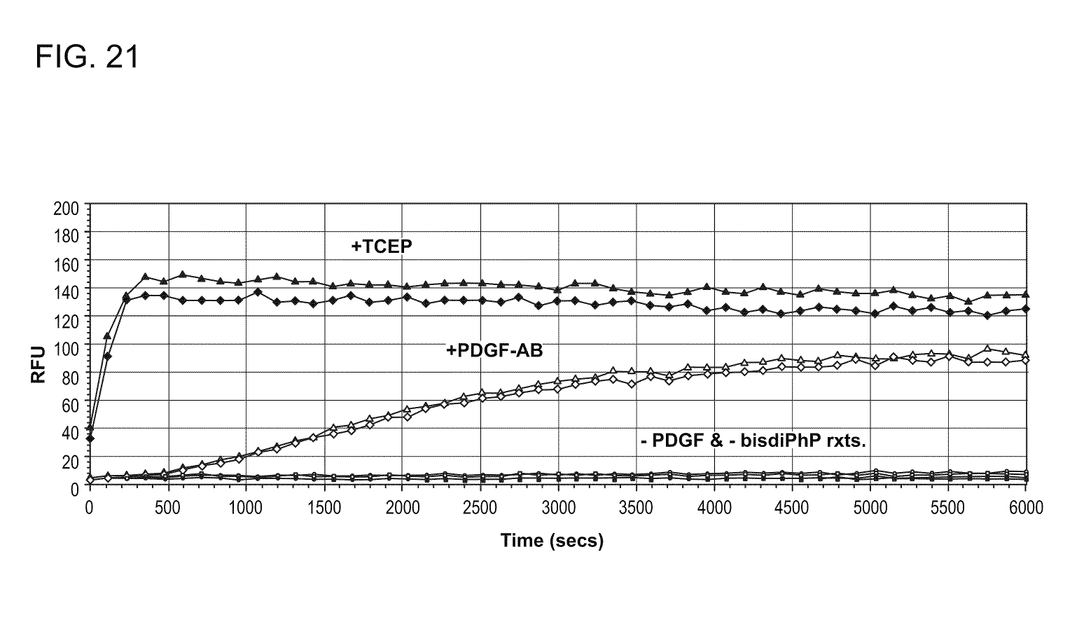

One DPC assay format investigated has detected the presence of protein platelet derived growth factor (AB subunits) or PDGF-AB using conjugates of anti-PDGF-A and anti-PDGF-B antibodies conjugated via zipcode and anti-zipcode sequences to reporter sequences of diphenylphosphine and DAZR, respectively (see, FIG. 20). In this reaction, anti-PDGF-B and anti-PDGF-A conjugates (0.15 μM) were incubated with 0.15 μM zip-coded DAZR oligonucleotide conjugate and 0.30 μM diphenylphosphine zip-coded oligonucleotide conjugate in the presence and absence of 0.15 μM of the target (PDGF-AB). The reaction mixtures contained 0.05 M sodium phosphate, pH 8 as buffer and were monitored over time at 30° C. in a microplate-based Fluorometer at 520 nm. FIG. 21 illustrates a typical time course of fluorescence generation of such a system in the presence of PDGF-AB. Negative controls lacked PDGF-AB or zipcoded bisdiphenylphosphine reactant. A positive control in the presence of a high concentration of excess TCEP (denoted “+TCEP”) indicates the maximum fluorescence that can be obtained if all the DAZR oligonucleotide was reduced to rhodamine.

FIG. 22 shows a schematic representation of an assay for the detection of PDGF-AB where the initial reaction product (rhodamine Green) is amplified by the amplification component (e.g., HRP) of the detection system. The assay, based on an ELISA format, used an anti-fluorescein antibody-HRP conjugate (Rockland, goat anti-fluorescein-HRP conjugate) which binds with sufficient affinity and specificity to rhodamine Green to discriminate rhodamine Green from the DAZR precursor. It was tested whether the antibody conjugate could be used to amplify the signal from DAZR reduction via DPC in the presence of PDGF-AB while discriminating the DAZR and diphenylphosphine precursors.

Briefly, the wells of an ELISA plate (a solid support) were coated with an anti-PDGF polyclonal antiserum. After incubating with PDGF, heterodimers of PDGF-AB were detected using two ligand-reporter assemblies (probes). Each of the ligand-reporter assemblies were based on the two molecule systems shown in FIG. 2. The Target binding component 1 comprises an anti-PDGF-A antibody covalently associated with a zip3 oligonucleotide, and the Target binding component 2 comprises an anti-PDGF-B antibody covalently associated with a zip2 oligonucleotide. The two target binding components were incubated with two reporter components denoted Reporter component 1 and Reporter component 2. Reporter component 1 contained an anti-zip3 oligonucleotide (which hybridizes to the zip3 oligonucleotide) covalently associated with DiPhP. Reporter component 2 contained an anti-zip2 oligonucleotide (which hybridizes to the zip2 oligonucleotide) covalently associated with DAZR. When the reporter 1 sequence of Reporter component 1 hybridizes to reporter 2 sequence of Reporter component 2, DiPhP is brought into reactive proximity with DAZR to reduce the DAZR molecule to produce rhodamine.

The resulting product was bound by an anti-fluorescein antibody-HRP conjugate (denoted anti-fluorescein-HRP). The HRP enzyme converts TMB into a colored product. Signal (Absorbance at 450 nm) was developed after incubation with Rockland goat anti-fluorescein-horse radish peroxidase conjugate with TMB substrate.

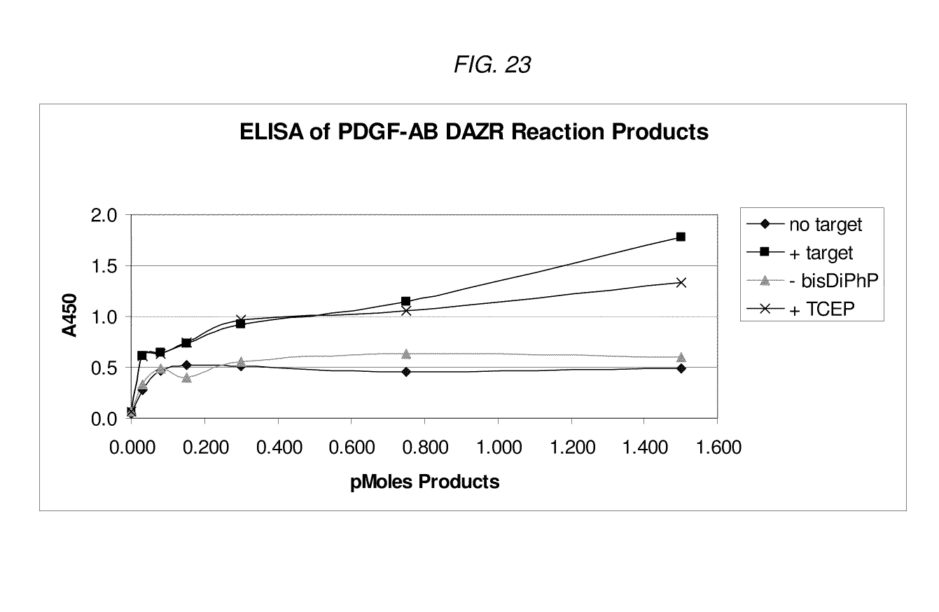

The DPC products generated at the end of the reaction shown in FIG. 22 were plotted as a function of the total picomoles of DAZR oligonucleotide from the reaction input into the ELISA (see, FIG. 23). As shown in FIG. 23, the ELISA response of the reaction mixture developed in the presence of PDGF-AB (denoted as +target) was similar to that achieved in the presence of TCEP (denoted +TCEP), both of which were much higher than the response developed in the absence of PDGF-AB (denoted as no target), or omitting the bisdiphenylphosphine oligonucleotide (denoted as −bisDiPhP). Under the conditions tested, the discrimination of the antibody between precursors and products was about five-fold, comparing reaction conditions that produced mostly reduced DAZR (Rhodamine Green) and with starting product (non-reduced DAZR).

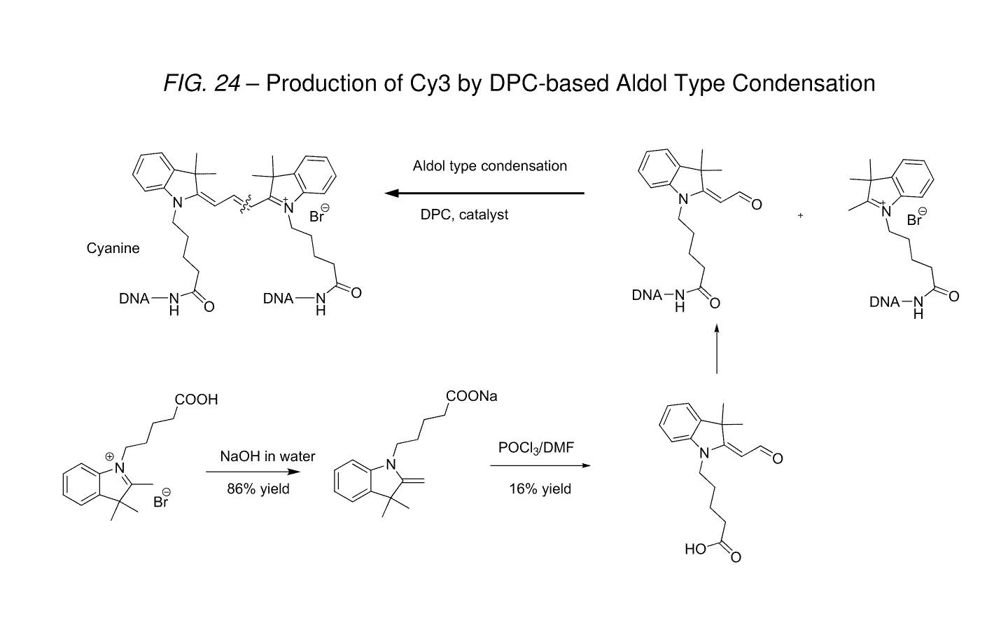

Antibodies have been developed against many classes of dyes, for example, rhodamines and coumarins. Another useful set of detection reagents are the non-fluorescent precursors indolinium and an indole aldehyde, and their fluorescent reaction product known as Cy3 (FIG. 24). The Cy3 reaction product can be detected by an anti-Cy3 antibody (Sigma Anti Cy3/Cy5 or Kreatech anti Cy3), neither of which bind to the indolinium or aldehyde precursors.

### Example 6

### Small Molecules Containing Epitopes

Numerous small molecules define epitopes for which antibodies have been developed. The antibodies usually are utilized as detection reagents, typically in immunoassays, for example, an ELISA format, for detecting, for example, toxins, pesticide residues, drugs etc. Epitopes that can be produced by DPC (i.e., where the products but not the precursors are bound by antibodies) and for which antibodies are commercially available (for example, from Santa Cruz) include, but are not limited to, amodiaquine, ampicillin, arginine, benzopyrene, biotin, cephalosporin, cloroquine, coumaric acid, digoxigenin, digoxin, ethenoadenosine, fluorescein isothiocyanate, FK506, glutathione, morphine, phencyclidine, theophylline, thioguanine.

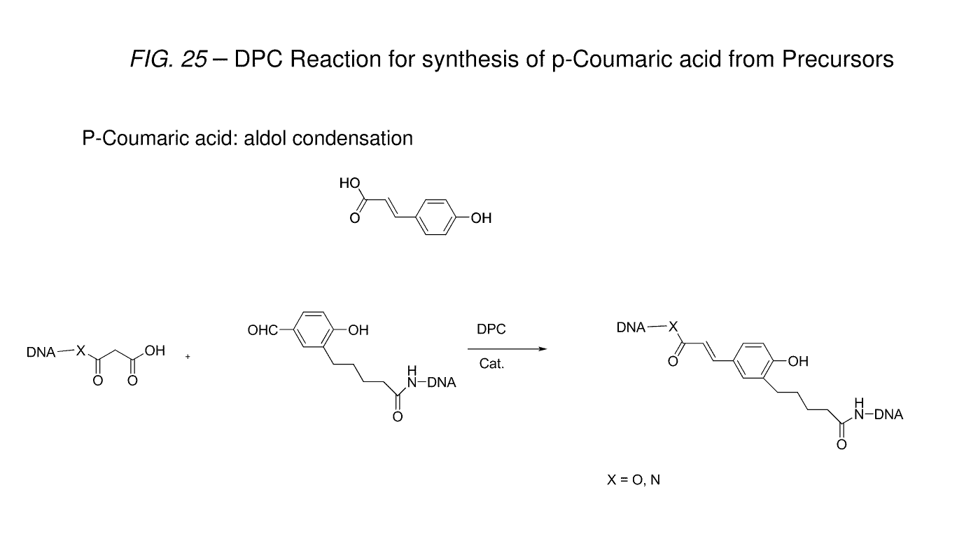

An exemplary synthetic scheme for the DPC mediated synthesis of p-coumaric acid by an aldol condensation is set forth in FIG. 25.

### Example 7

### Detection of EGFR Dimers

This Example describes an assay for detecting the presence and/or amount of EGFR dimers using EDC-sNHS DPC to produce an epitope. The production of detectable signal depends upon the presence of receptor dimers and the assay effectively discriminates the constituent monomers.

EGF-activated A431 cells were washed by centrifugation three times in phosphate buffered saline (“PBS”; Sigma Chemical Company, St. Louis, Mo.). 50,000 cells were introduced into each well of a hi-bind plate in PBS and allowed to settle overnight at 4° C. The immobilized cells were washed three times with PBS. The wells were blocked with Blocking Solution (PBS-T1 mg/mL bovine serum albumin (BSA) 0.1 mg/mL rabbit IgG) for 1 hour at room temperature, then washed three times with PBS plus Tween-20 (“PBS-T”; Sigma Chemical Company), once with water and dried at room temperature.

The wells then were incubated with equal amounts of anti-EGFR (Labvision, Fremont, Calif.) conjugated to the amino terminus of Zip2 (anti-EGFR-Zip2; 0 to 15 pMoles) and anti-EGFR (Labvision) conjugated to the amino terminus of Zip5 (anti-EGFR-Zip5; 0 to 15 pMoles) for one hour and washed 3 times with PBS-T. The wells then were incubated with 20 pMoles each of antzip2_T7_p1 and antzip5_T7_p2 and washed 3 times with PBS-T. Wells containing antizip-T7 hemipeptides conjugates then were incubated with a solution of 0.2 M EDC, 0.15 M sulfo-NHS in 0.1 M 4-morpholineethansulfonic acid (MES), pH 6.5 buffer (EDC-sulfo-NHS) for 30 minutes. A second incubation and wash with Blocking Solution was then conducted.

Samples then were incubated with 100 μL of 6.6 μM anti-T7-HRP conjugate in Blocking Solution for one hour. The wells were incubated with Amplex Red in accordance with the manufacture's instructions and fluorescence was monitored with excitation at 530 nM and emission at 585 nM with a Molecular Devices Fluorescent Microplate reader. Controls included samples incubated without anti-EGFR conjugates; samples incubated without T7 hemipeptide conjugates; and samples incubated without both anti-EGFR conjugates and T7 hemipeptide conjugates.

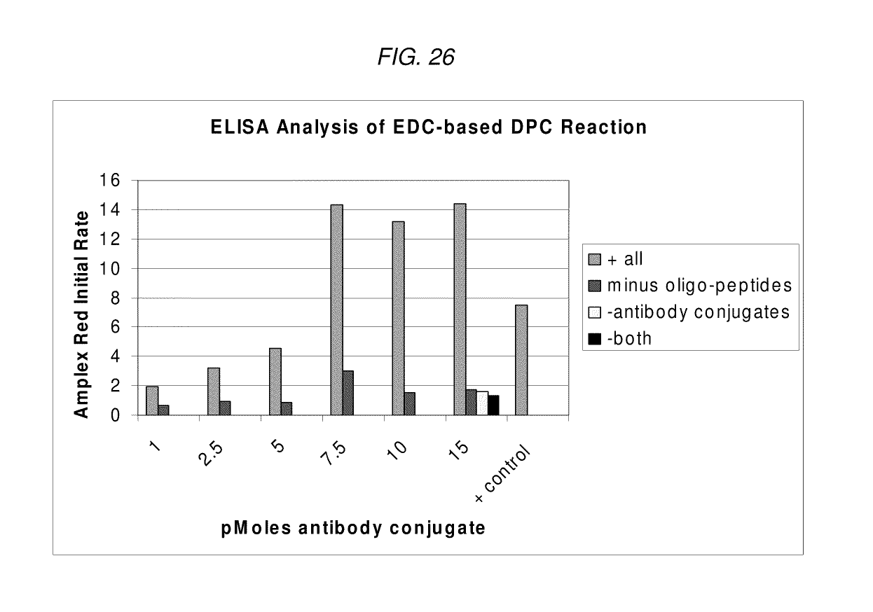

The results are shown in FIG. 26. Reaction rates in the linear phase of the assay increased as a function of full-length T7 epitope on the cells.

### Example 8

### Flow Cytometric Assay for EGFR and ErbB2 Dimers

Adherent A431 cells were serum starved overnight and then washed and detached from the plate by tryptic digestion. Suspensions of A431 cells were either left untreated or treated with EGF (200 ng/mL) on ice for 15 min. Cells then were fixed by incubation with 2% formaldehyde for 20 minutes on ice. Endogenous peroxidase activity was quenched by incubation with 3% hydrogen peroxide in PBS for 5 minutes at room temperature. Cells were blocked with non-specific rabbit IgG (100 μg/mL) and tRNA (1 μM) in PBS containing 2% BSA and 5% dextran sulfate and then incubated with a solution containing 2% BSA, 5% dextran sulfate, 100 μg/mL rabbit IgG, 1 μM tRNA in PBS, and the following antibody-zipcode conjugates and anti-zipcode T7 hemipeptides.

For the EGFR homodimer assay, cells were incubated with 5 μg/mL each of the antibody conjugates egfr1 antibody-Zip2 and egfr1 antibody-Zip5, and 60 nM each of antzip2_T7_p1 and antzip5_T7_p2.

For the EGFR-ErbB2 heterodimer, cells were incubated with 5 μg/mL each of egfr1 antibody-Zip5 and 200 nM anti-ErbB2 affibody conjugated to the amino terminus of the zipcode2 reporter, and 60 nM each of antzip2_T7_p1 and antzip5_T7_p2.

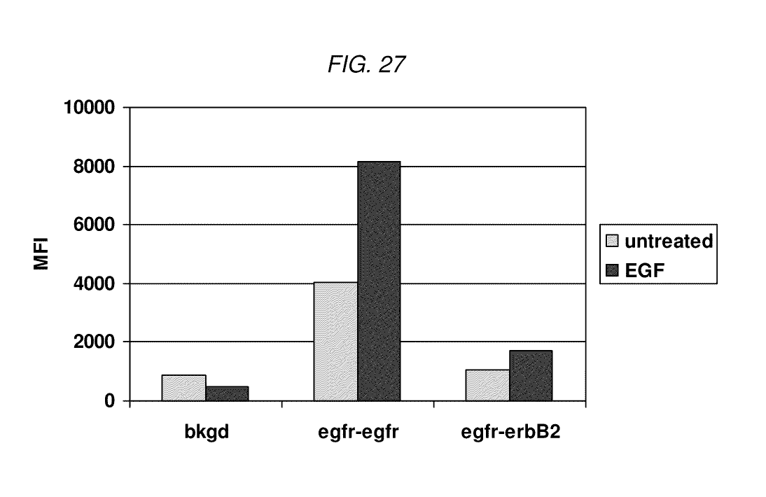

The reagents were incubated with the cells for 1 hour at 30° C. To each assay was added EDC-sulfo-NHS for 1 hour at room temperature to ligate any resultant hemi-peptides brought into reaction proximity via hybridization of the complementary portions of the anti-zipcode sequences. Then 1 μg/mL rabbit anti-T7 antibody (Novus Biologicals, Littleton, Colo.) conjugated with horseradish peroxidase was added to each assay and incubated in the assay mixture for 1 hour at room temperature. Tyramide-Alexa568 (Invitrogen, Carlsbad, Calif.) then was added and the mixture incubated for 5 minutes at room temperature. The Alexa568 labels were covalently attached by the HRP in the rabbit anti-T7 antibody-HRP complex. The cells were analyzed by flow cytometry for the presence of Alexa568 label, and the results are summarized in FIG. 27.

As seen in FIG. 27, the mean fluorescence intensity (MFI) obtained for the T7 peptide ligation DPC assay for the EGFR homodimer was significantly greater than background (denoted bkgd). The background value was determined as described above but lacking one of the anti-EGFR antibodies. The MFI for the EGFR homodimer increased in response to EGF treatment (denoted EGF). These results were consistent with the presence of a basal level of EGFR homodimer in the untreated cells and induction of further homodimerization in response to exogenous EGF. In contrast, the MFI for the EGFR-ErbB2 heterodimer in untreated cells was not significantly above the background suggesting very little or no heterodimer was present at the basal level. However, in response to EGF, the DPC signal for EGFR-ErbB2 was elevated above the background suggesting that the heterodimer was induced by EGF.

These results are consistent with the accepted mechanisms of action of cells in response to EGF. In A431 cells, EGFR is highly expressed, while ErbB2 is expressed at a much lower level. The magnitude of the MFI signals detected in this assay was consistent with these expression levels.

### Example 9

### DPC Assay for Bcr-Abl

Bcr-Abl is an abnormal fusion oncoprotein expressed in chronic myelogenous leukemia (CML). Detection of Bcr-Abl and discrimination of individual Bcr and Abl can be important for the diagnosis of CML, as well as detection of minimal residual disease after treatment with therapeutic agents such as Gleevec® (imatinib mesylate). The CML cell line, KY01, expresses the Bcr-Abl fusion protein as well as the individual Bcr and Abl proteins.

The protocol for T7 peptide ligation DPC assay for Bcr-Abl was similar to that described in Example 8, except that the antibody-zipcode conjugates used were anti-Bcr antibody 7C6 covalently coupled to the amino terminus of Zip2 and anti-Abl antibody 19-110 covalently coupled to the amino terminus of Zip5 (19-110-Zip5). In addition, because Bcr-Abl is an intracellular protein, the cells were permeabilized to allow penetration of the DPC assay reagents into the cells. Permeabilization was accomplished by addition of 0.3% saponin to all solutions used in the assay beginning with the formaldehyde used to fix the cells. Other than these modifications, the same protocol described in Example 8 was used to detect Bcr-Abl in KY01 cells.

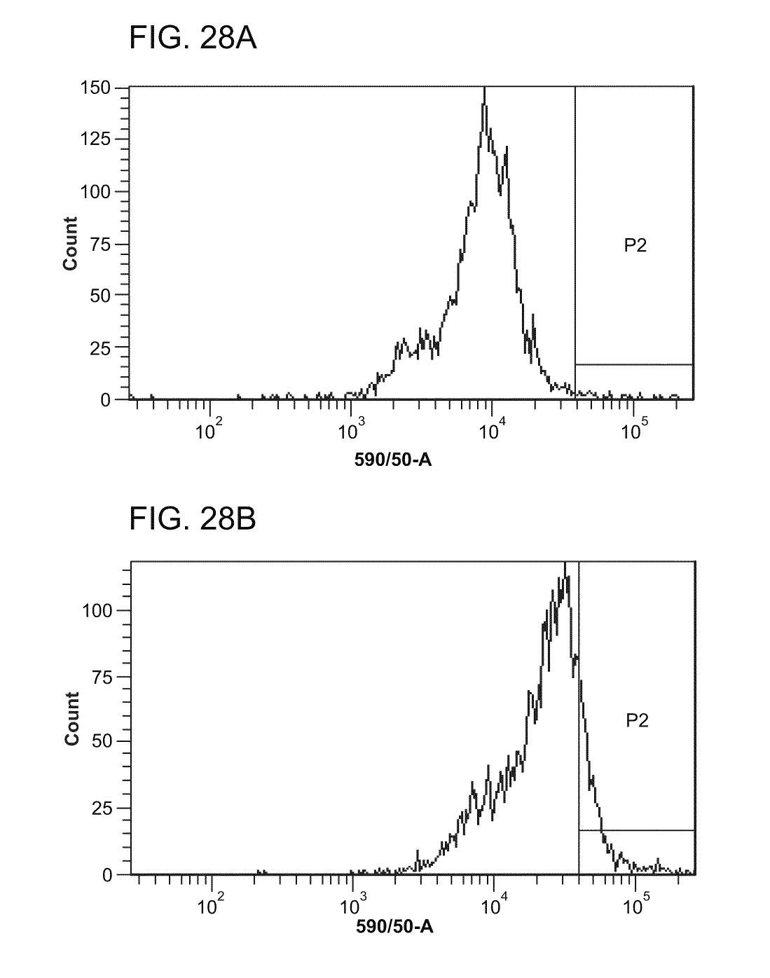

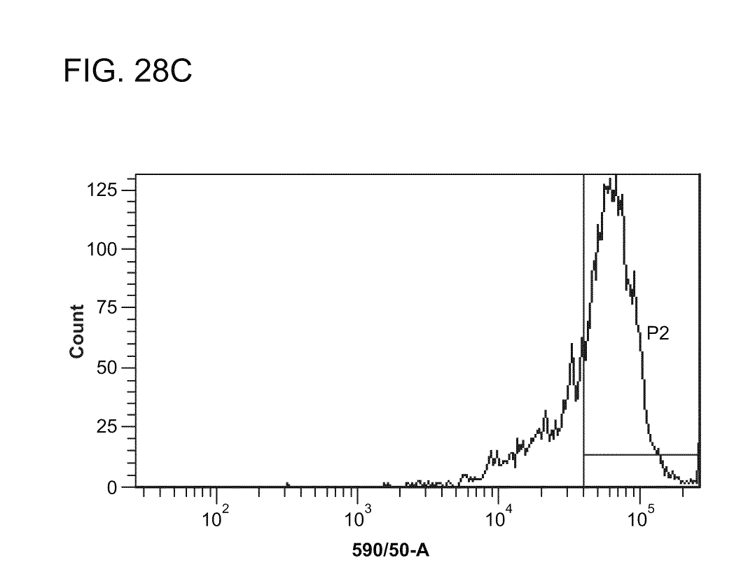

The results from the flow cytometric analysis of three assay samples are summarized in FIG. 28. FIG. 28A shows the MFI distribution of KY01 cells blocked with non specific IgG and exposed only to rabbit anti-T7-HRP; in effect the background due to non-specific binding of this antibody to KY01 cells. FIG. 28B shows the MFI distribution of the negative control sample that did not contain the anti-Abl antibody conjugate but otherwise contained all the other reagents necessary for a DPC reaction. The MFI of this negative control is slightly elevated over that of the anti-T7-HRP background. FIG. 28C shows the DPC signal indicating the presence of Bcr-Abl. This sample contained both antibody-zipcode conjugates as well as the other reagents necessary for the DPC ligation and detection of the intact T7 peptide. The MFI distribution is higher than any of the controls.

### Example 10

### DPC Detection of Bcr-Abl in a CML-Derived Cell Line and in a CML Patient Bone Marrow Sample

KY01 cells were grown in 10% RPMI 1640 medium with 10% bovine fetal calf serum. Purified CML patient bone marrow mononuclear cells were freshly harvested and purified or alternatively obtained from a cryopreserved sample. About 200,000 cells were washed in PBS, followed by incubation with 0.25 mL Permeabilization/Fixation buffer (Becton-Dickinson, Franklin Lakes, N.J.) for 20 minutes at room temperature to fix the cells. The cells then were washed in 1% BSA/PBS and then incubated with 0.2 mL of Permeabilization/Wash buffer (Perm/Wash) (Becton-Dickinson, Franklin Lakes, N.J.) containing 3% hydrogen peroxide for 10 minutes at room temperature. The cells then were washed 2 times with Perm/Wash buffer and then blocked in Perm/Wash buffer containing 50 nM of a mixture of non-specific 59 mer oligonucleotides for 30 minutes.

At room temperature, a mixture (0.1 mL) containing an anti-Bcr antibody B12 covalently associated with the amino terminus of Zip2, an anti-Abl antibody 19-110 covalently associated to the amino terminus of Zip5 together with antzip2_T7_p1 and antzip5_T7_p2 in a Perm/Wash buffer containing 50 nM of the blocking oligomer mix was prepared. The mixture was added to the cells, and the cells then were incubated for 1 hour on ice. The cells then were washed with Perm/Wash buffer, and incubated with 0.3 mL EDC-sulfo-NHS containing 0.2% saponin at room temperature for 1 hour.

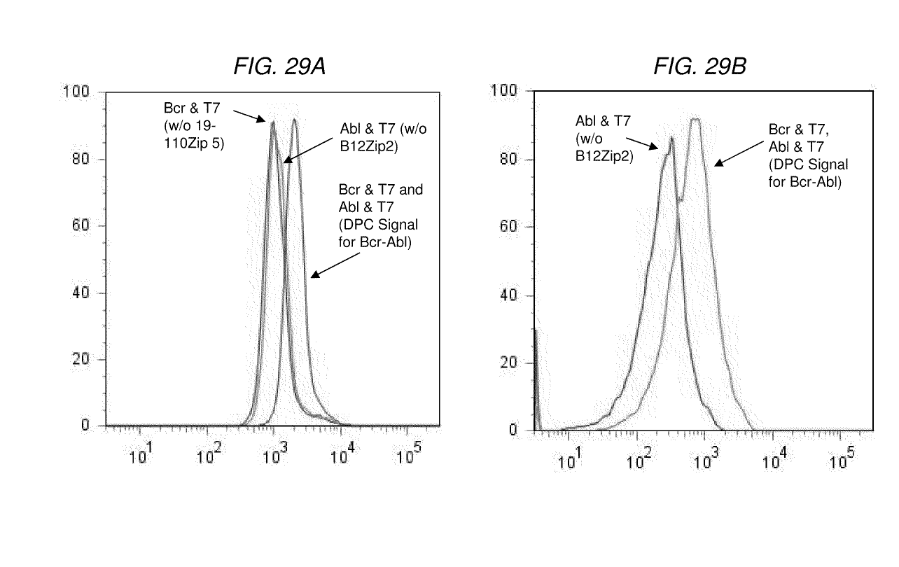

The cells were washed once with Perm/Wash buffer, blocked with 200 μg/mL normal human IgG for 30 minutes, and then incubated with rabbit anti-T7 antibody (Novus Biologicals, Littleton, Colo.) covalently associated with HRP for 1 hour. The cells then were washed with Perm/Wash buffer, and incubated with a goat anti-rabbit IgG F(ab′)₂ fragment covalently associated with Alexa568. The antibody was diluted 1:2000 in Perm/Wash buffer containing 200 μg/mL normal goat IgG prior to use. The reaction volume was 0.2 mL. The cells then were washed twice with Perm/Wash buffer and analyzed by flow cytometry. The results are shown in FIG. 29.

FIG. 29 shows the results obtained by this method on KY01 cells (FIG. 29A) and CML patient mononuclear cells (FIG. 29B). For each of the cell types, the signal associated with the presence of Bcr-Abl was significantly above the background controls. The controls included samples in which either the Abl antibody 19-110Zip5 or the Bcr antibody B12Zip2 was not included in the reactive mixture.

### Example 11

### Detection of ErbB2 Homodimers in Breast Cancer Tissue

Breast ductal carcinoma tissue sections (4 microns thick; BioGenex, San Ramon, Calif., Catalog No. FG-134M) were deparaffinized in three changes of xylene and rehydrated in graded series (90%, 80% and 70%) of ethyl alcohol. Epitope retrieval for ErbB2 was performed in 10 mM sodium citrate buffer (pH6.0) for 20 minutes in a microwave oven. Endogenous HRP activity was blocked in 3% H₂O₂ in PBS. After washing, sections were blocked for 1 hour at 4° C. in Blocking Buffer. During incubation, slides were kept in a humidified chamber.

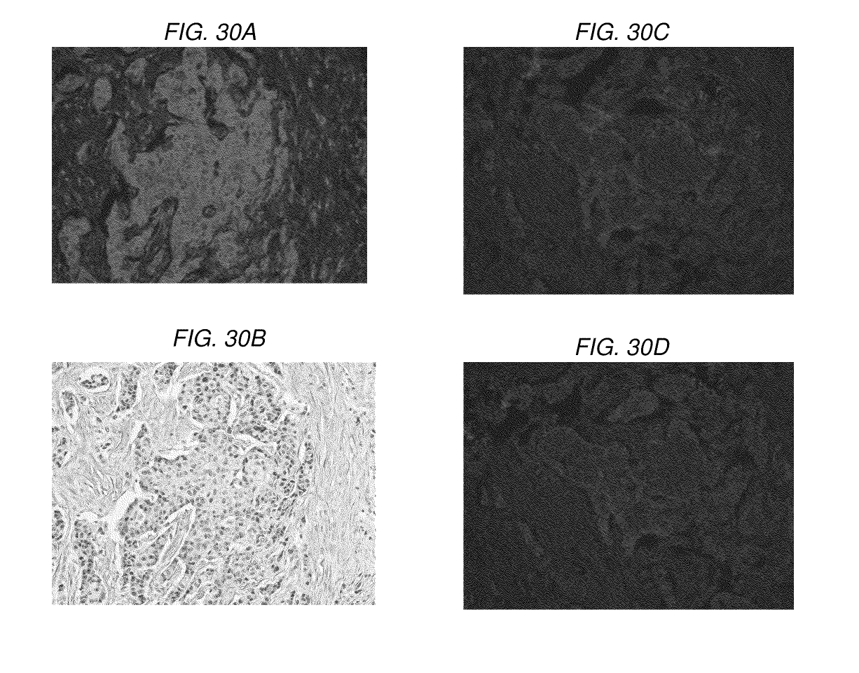

A mixture of an anti-ErbB2 9G6 antibody (Santa Cruz Biotechnology, Santa Cruz, Calif.) covalently associated with Zip2 and anti-ErbB2 9G6 antibody covalently associated with Zip5 (60 nM each in Binding Buffer), was applied to the sections for 30 minutes at room temperature. After washing with Binding Buffer, the sections were incubated for 1 hour at room temperature with 180 nM each of antzip2_T7_p1 and antzip5_T7_p2 in Binding Buffer. After two washes with PBS and one wash with 0.1M MES/150 mM NaCl buffer, T7 peptide ligation was performed in EDC-sulfo-NHS for 2 hours at room temperature. After washing with PBS, the slides were blocked in T7-blocking buffer (PBS, 2% BSA, 0.1 mg/mL lactalbumin, 0.1 mg/mL rabbit IgG) for 45 minutes at room temperature and then incubated with anti-T7-HRP antibody (Novus Biologicals, Littleton, Colo.) at 2 μg/mL in T7-blocking buffer for an additional 45 minutes at room temperature. The slides then were labeled with Tyramide-AlexaFluor568 (TSA KIT, Molecular Probes, Carlsbad, Calif.) for 5 minutes at room temperature, washed in PBS, mounted in ProLong Gold antifade mounting medium (Molecular Probes, Carlsbad, Calif.) and stored at 4° C. Microscope analysis was performed on epifluorescent Nikon ET-2000U microscope equipped with bandpass, 510-550 nm, excitation and longpass, 580 nm, emission filters. The results are shown in FIG. 30.

Specific staining of ErbB2 homodimer was observed only in the ductal cells and was localized to the plasma membrane (FIG. 30A). FIG. 30B is a corresponding HE stained section shown to reveal the tissue architecture. FIGS. 30C and 30D are negative controls lacking 9G6-Zip5 conjugate (FIG. 30C) or both the 9G6-Zip2 and the 9G6-Zip5 conjugates (FIG. 30D).

### Example 12

### Production and Use of T7 Modified Hemipeptides Using Native Chemical Ligation

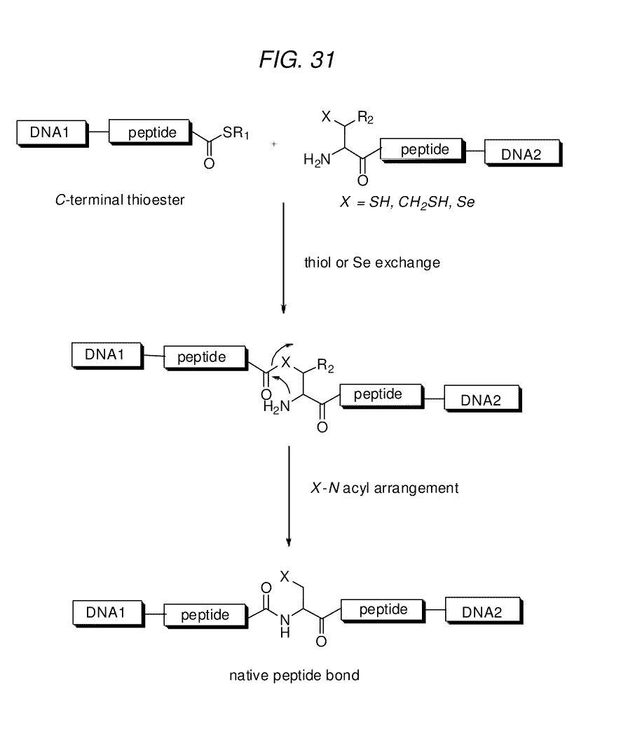

Native chemical ligation (NCL) involves the condensation and rearrangement of the reaction of a thioester-substituted carboxyl group of one peptide with an N-terminal cysteine amino acid (or cysteine analog) in another peptide to ligate the two together to produce a native peptide bond. The potential advantage of this chemistry is that no external catalyst is required, greatly reducing the extent of nonspecific reactions to the target protein or cell. Furthermore, with this chemistry any number of carboxyl and amino side chain containing amino acids are permitted in the epitope because they typically do not lead to side reactions. The general chemistry used in this Example is set forth in FIG. 31.

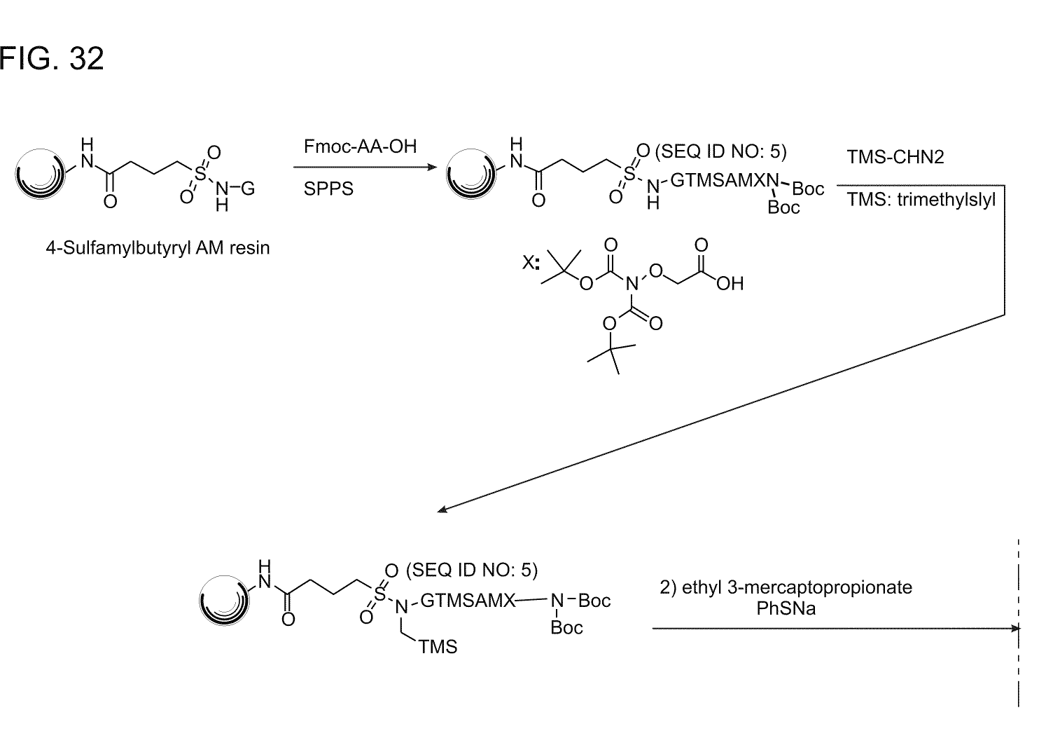

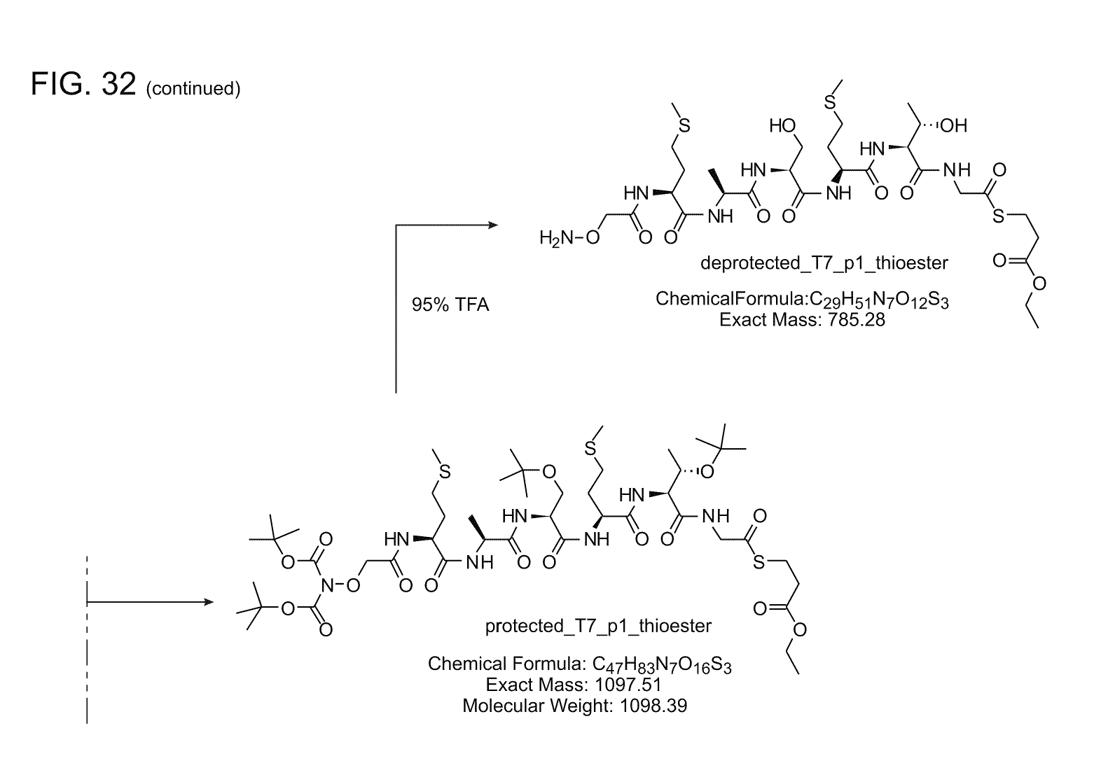

The synthesis of one T7 P1 hemipeptide thioester is set forth in FIG. 32. Briefly, T7 P1 hemipeptide was first assembled on a preloaded acylsulfonamide safety-catch resin (NovaBiochem, Gibbstown, N.J.) by standard Fmoc protocols. A hydroxylamine functional group was incorporated into the N-terminus to allow for DNA conjugation through the coupling of bis-Boc(tert-butoxycarbonyl) protected amino oxyacetic acid. After the synthesis, the resin was activated by trimethylsilyldiazomethane (TMS-CHN₂) and then treated with ethyl 3-mercaptopropionate and a catalytic amount of NaSPh. This converted the carboxy terminal glycine in T7 P1 to a thioester. The MS data indicated the crude cleavage mixture contained mainly the protected T7 P1 thioester. Further TFA treatment and HPLC purification afforded the fully deprotected T7 P1 thioester. An alternate route to the synthesis of a T7 P1 thioester is set forth below in Example 17.

A modified T7 P2 hemipeptide contained a Gly to Cys mutation and the sequence GMQQC-NH₂ (SEQ ID NO: 72). The T7 P1 hemipeptide thioester was conjugated to antizip2 to create antizip2_T7_p1 thioester and the modified T7 P2 hemipeptide was conjugated to antizip5 to create antizip5_T7_p2_Cys, as described previously. Conjugation of the T7 P2 hemi-peptide to antizip5 was performed in the presence of 2 mM DTT to prevent oxidation of the cysteine residue. The antizip conjugated hemi-peptides (0.5 μM each) were mixed together in 2% thiophenol, 50 mM NaPi, 150 mM NaCl, pH 6.0 overnight. Gel electrophoresis of the reaction product demonstrated the production of the Cys-containing T7 peptide. The T7 peptide product could also be prepared when 0.5 mM DTT was substituted for 2% thiophenol. The anti-T7 antibody used in the previous experiments recognized the Cys-modified T7 peptide, but did not bind to either the T7_p1_S(Et3 MP) hemi-peptide or the T7_p2_Cys hemi-peptide.

Unlike the EDC/sNHS peptide ligation protocol, NCL utilizes two hemi-peptides that require no additional reagent to form a peptide bond. Accordingly, an adjustment in either the assay protocol or in the melting temperature (Tₘ) of the reporter oligonucleotides associated with each of the hemi-peptides may be required to ensure product formation only occurs in the presence of target-dependent oligonucleotide duplex formation. In one embodiment, one of the antizip-reporter-hemipeptide constructs was added to the reaction mixture and incubated for a period of time sufficient incubation to allow for specific binding to the target. The reaction mixture then was washed to remove any unbound constructs and then the second antizip-reporter-hemipeptide construct was added.

For the EDC/sNHS peptide ligation protocol, the reporter oligonucleotides typically are 10 bases long and are 100% complementary. Such reporter oligonucleotides have a melting temperature (Tₘ) of greater than about 25° C. However, for the NCL protocol, the reporter oligonucleotides can result in peptide formation without specific binding to the target. Nevertheless, non-specific ligation can be reduced by lowering the Tₘ below 25° C. Thus, in one embodiment, the reporter oligonucleotides are modified so that they have a Tₘ from about 8° C. to about 25° C. The reporter oligonucleotides preferably have a Tₘ from about 9° C. to about 20° C. When the length of the individual reporter oligonucleotides differ, the longer reporter oligonucleotide typically is associated with the hemi-peptide bearing the thioester terminus and the shorter reporter oligonucleotide is associated with the hemi-peptide bearing the thiol (i.e. cysteine or cysteine analog) terminus.

Modification of the Tₘ of the reporter oligonucleotides can be achieved by shortening the length of one or both of the reporters, altering salt conditions and other reagents in the reaction, and/or by introducing mismatches that reduce complementarity of the reporter sequence below 100%. The Tₘ of two oligonucleotides can be estimated based upon sequence length and content according to known mathematical formula using the methods set forth in Panjkovich, A. et al., Bioinformatics 2005, 21(6):711-22 and Panjkovich, A. et al., Nucl. Acid Res. 2005, 33:W570-W572. Alternatively, determining the Tₘ of any two reporter oligonucleotides utilized in the present invention is achieved by experimental testing well known in the art.

### Example 13

### Use of T7 Modified Hemipeptide-Antizip Conjugates Containing Lower Tm Reporter Oligonucleotide Portions to Detect DNA Targets Via NCL

5 pMoles of antizip2_T7_p1-S(Et3 MP) containing various reporter oligonucleotide portions and antizip5_T7_p2-Cys containing various reporter oligonucleotide portions were added in 100 μL of 50 mM sodium phosphate (pH 6), containing 5% w/v dextran sulfate. Wells containing 5 pMoles of biotin-zip5 or a combination of 2.5 pMoles each of biotin-zip2 and biotin-zip5 immobilized on streptavidin plates were tested. The wells were incubated for 30 minutes at 25° C. and then washed three times with PBS-T buffer. The reactions were incubated with 100 μL of 0.01 μM mouse anti-T7-alkaline phosphatase conjugate (Novagen, Gibbstown, N.J.) in PBS-T buffer 30 minutes at 25° C., and washed four times with 200 μL PBS-T buffer. The reaction mixtures were developed with Attophos detection solution (Amersham, Piscataway, N.J.) and the kinetics of fluorescence development monitored (excitation 435 nM/emission at 585 nM on a Molecular Devices Fluorescence plate reader at 25° C.). The rate of increase of fluorescence emission (ΔF/sec) within the linear range (typically between 0 and 600 seconds) was plotted against each experimental condition.

The various individual antizip2 and antizip5 sequences used in this experiment are set forth in TABLE 2.

TABLE 2

Antizip Sequences with Unmodified and Modified Reporter Oligonucleotides

| | | SEQ ID |
|---|---|---|
| NAME | Sequence | NO. |
| Antizip5_10mer (Cys full length) | NH₂-CGAATTTATA-X-CTGACTATGGATGGCACG | 68 |
| Antizip2_10mer (TE 10-mer) | GGACTCGAGCACCAATAC-X-TATAAATTCG-NH₂ | 66 |
| Antizip5_2mismatch (Cys 10-mer 2 | NH₂-CCAATTAATA-X-CTGACTATGGATGGCACG | 73 |
| MM) | | |
| antizip5_1mis (Cys 10-mer 1 MM) | NH₂-CCAATTAATA-X-CTGACTATGGATGGCACG | 74 |
| antizip2_9mer (TE 9-mer) | GGACTCGAGCACCAATAC-X-TATAAATTC-NH₂ | 75 |
| antizip2_8mer (TE 8-mer) | GGACTCGAGCACCAATAC-X-TATAAATT-NH₂ | 76 |
| antizip5_9mer (Cys 9-mer) | NH₂-GAATTTATA-X-CTGACTATGGATGGCACG | 77 |
| antizip5_8mer (Cys 8-mer) | NH₂-AATTTATA-X-CTGACTATGGATGGCACG | 78 |

X = Spacer Phosphoramidite 18 (Glen Research Sterling VA, USA)

UV melting curves were obtained with sample solutions made by combining 1 μM of each of the modified antizip5 and antizip2 reporter DNAs in PBS buffer (10 mM sodium phosphate, 154 mM NaCl, pH 7.4) at ambient temperature. All measurements were conducted in a 1-cm path length quartz cell (total 1200 μL) with a magnetic stir bar inside. The absorbance at 260 nm was recorded as a function of temperature using a Cary 300 Bio UV-Vis spectrophotometer equipped with a Peltier system thermocontroller with heating/cooling rates of 0.5° C./minute over the range of 0 to 75° C. Dry N₂ gas was passed through the spectrophotometer sample chamber to prevent moisture condensation below ambient temperature.

Data were collected and the resulting curve was smoothed. Then the smoothed melting curve was fitted to the two-state model with sloping base lines using a nonlinear least-square program MeltWin 3.0 (McDowell, J A et al, Biochemistry 1996, 35:14077-14089). The melting temperature, Tₘ (defined as the temperature at which 50% of a complex is dissociated into its constituent components) was determined from the inflection point maximum of the first derivative of the melting curves. If the binding buffer contains dextran sulfate, the buffer melting curve was subtracted from the sample before conducting the fitting.

The specific antizip pairs used in this experiment and their experimentally determined melting temperatures are shown in TABLE 3. When more than one Tₘ is shown for a particular Antizip pair, it reflects the results of multiple Tₘ determinations.

TABLE 3
Tₘ of Antizip Pairs

| Antizip Pair | Tₘ (° C.) |
|---|---|
| Antizip5_10mer/antizip2_10mer | 26.0, 25.6, 25.5 |
| Antizip5_9mer/antizip2_10mer | 17.0 |
| Antizip2_9mer/antizip5_10mer | 15.5 |
| Antizip5_2mismatch/antizip2_10mer | 14.1, 14.0 |
| Antizip5_9mer/antizip2_9mer | 11.0 |
| Antizip5_1mis/antizip2_10mer | 8.9, 8.5, 9.1 |
| Antizip5_8mer/antizip2_10mer | 8.1 |
| Antizip2_10mer/antizip5_8mer | 8.1 |
| Antizip5_8mer/antizip2_8mer | 7.2 |

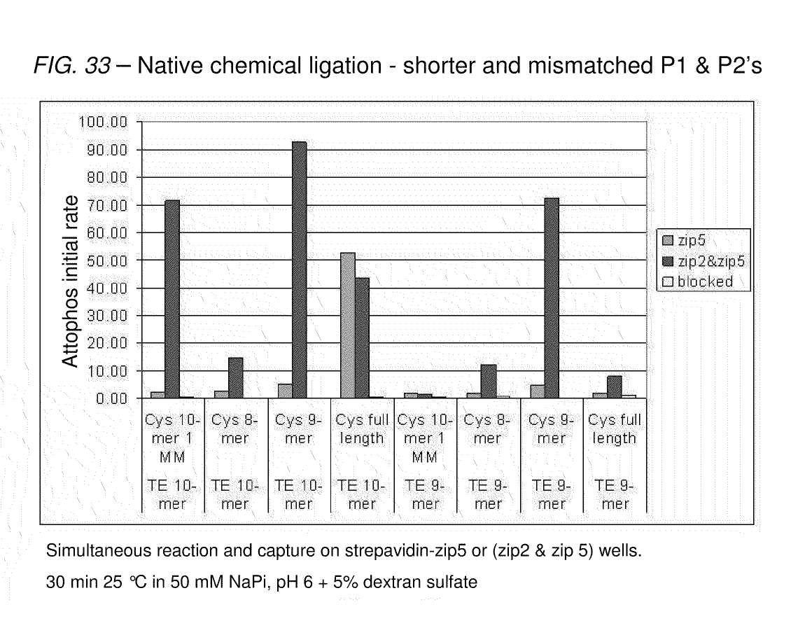

The results of this study depicted in FIG. 33 demonstrate that, in general, reporter oligonucleotide pairs having a Tₘ of less than 25° C. and greater than about 8° C. result in specific hemi-peptide ligation. Without wishing to be bound by theory, it is contemplated that the low signal observed for the Antizip2_{—}9 mer/antizip5_{—}10 mer pair relates to the geometry created between the two hemi-peptides prevented efficient ligation. The antizip5_{—}9 mer/antizip2_{—}10 mer pair, which showed a similar Tₘ gave a very strong specific signal. Thus, it may be important when the reporter oligonucleotides are of different length that the longer reporter bear the thioester terminus and the shorter reporter bear the thio terminus of the cysteine or cysteine analog.

### Example 14

### Additional Cys-Substituted Mutant Peptides and Hemi-peptide Pairs

Cysteine incorporation was tested at other positions in T7. Cysteine incorporation in T7 at Gly7, Gln8 and Gln 9 each produced peptides that were recognized by anti-T7 antibodies and non-reactive hemipeptides. Accordingly, the invention provides mutant T7 peptides and hemi-peptide pairs denoted P1 hemi-peptide and P2 hemi-peptide are shown in TABLE 4. For each peptide and P2 hemi-peptide set forth below, cysteine is optionally replaced with a cysteine analog.

TABLE 4
Mutant T7 Peptides and Corresponding Hemi-
peptides

| | SEQ | | SEQ | P2 | SEQ |
|---|---|---|---|---|---|
| Mutant T7 | ID | | ID | Hemi- | ID |
| Peptide | NO. | P1 Hemi-peptide | NO. | peptide | NO. |
| MASMTCGQQMG | 38 | MASMT-thioester | 50 | CGQQMG | 58 |
| MASMTGCQQMG | 39 | MASMTG-thioester | 5 | CQQMG | 72 |
| MASMTGGCQMG | 40 | MASMTGG-thioester | 51 | CQMG | 79 |
| MASMTGGQCMG | 41 | MASMTGGQ-thioester | 52 | CMG | 80 |

Similar NCL strategies have been employed for peptides that are detected by other means, such as ligand binding (StrepTag peptide detected by binding to either streptavidin or strepactin). For StrepTag, Cys substitution at any of Ser₃, His₄, Pro₅ and Gln₆ produced a peptide that was recognized by StrepTactin and non-reactive hemipeptides. Thus the invention provides the mutant StrepTag peptides and corresponding hemi-peptide pairs denoted P1 hemi-peptide and P2 hemi-peptide are shown in TABLE 5 below (P1 and P2 hemi-peptide in the same row), which are useful in and are a part of this invention. For each peptide and P2 hemi-peptide set forth below, cysteine is optionally replaced with a cysteine analog.

TABLE 5

Mutant StrepTag Peptides and Corresponding Hemi-peptide Pairs

| | SEQ ID | | SEQ ID | | SEQ ID |
|---|---|---|---|---|---|
| StrepTag Peptide | NO. | P₁ Hemipeptide | N0. | P2 Hemipeptide | NO. |
| (G)₀₋₂-NWCHPQFE-(G)₀₋₂ | 42 | (G)₀₋₂-NW-thioester | 53 | CHPQFE-(G)₀₋₂ | 59 |
| (G)₀₋₂-NWSCPQFE-(G)₀₋₂ | 43 | (Gly)₀₋₂-NWS-thioester | 54 | CPQFE-(G)₀₋₂ | 60 |
| (G)₀₋₂-NWSHCQFE-(G)₀₋₂ | 44 | (Gly)₀₋₂-NWSH-thioester | 55 | CQFE-(G)₀₋₂ | 81 |
| (G)₀₋₂-NWSHPCFE-(G)₀₋₂ | 45 | (Gly)₀₋₂-NWSHP-thioester | 56 | CFE-(G)₀₋₂ | 82 |

Similar NCL strategies have been used for peptides that are detected by enzyme activation (e.g., S-15 peptide, which is required to activate a deletion mutant of ribonuclease S-protein). For S-15, Cys substitution at Glu9 produced a peptide that activated the ribonuclease mutant and N-terminal inactive hemi-peptides. Thus the invention provides the mutant S-15 peptide and corresponding hemi-peptide pair shown in TABLE 6 below, which are useful in and are a part of this invention. For the peptide and P2 hemi-peptide set forth below, cysteine is optionally replaced with a cysteine analog.

TABLE 6
Mutant S-15 Peptide and Corresponding Hemi-
peptide Pairs

| | SEQ | | SEQ | P₂ | SEQ |
|---|---|---|---|---|---|
| | ID | P₁ | ID | Hemi- | ID |
| S-15 Peptide | NO. | Hemi-peptide | NO. | peptide | NO. |
| KETAAAKFCRQHMDS | 47 | KETAAAKF- | 57 | CRQHMDS | 61 |
| | | thioester | | | |

Each hemi-peptide set forth in TABLES 4-6 is a reactive peptide fragment according to this invention. As such, any hemi-peptide may be conjugated to a reporter oligonucleotide sequence. Similarly, the peptides set forth in TABLES 4-6 may also be conjugated to a reporter oligonucleotide sequence. The hemi-peptide and peptide-reporter oligonucleotide sequence is optionally further conjugated to an antizip sequence.

In one embodiment, the invention provides a kit comprising a first ligand-reporter assembly comprising a first hemi-peptide pair member set forth in TABLES 4-6 associated with a first reporter oligonucleotide sequence, and a second ligand-reporter assembly comprising the corresponding hemi-peptide pair member associated with a second reporter oligonucleotide, wherein the first and second reporter oligonucleotide sequences are sufficiently complementary to one another to hybridize with a Tₘ from about 8° C. to about 25° C. Each ligand-reporter assembly may optionally comprise one or more additional components selected from a zip or antizip oligonucleotide sequence, a spacer oligonucleotide sequence, and a binding moiety having binding affinity to a biological target.

The ligand-reporter assemblies can be a single molecule. Alternatively, the ligand reporter assemblies can comprise two or more components (for example, a reporter component and target binding component) that are non-covalently associated with one another to create a functional probe. In certain embodiments, each probe component comprises an antizip oligonucleotide sequence but lacks a binding moiety. In certain embodiments, each probe component comprises a different antizip oligonucleotide sequence but lacks a binding moiety.

In certain embodiments, the kit further comprises one or more binding components comprising a binding moiety having binding affinity to a biological target covalently or non-covalently associated to zip oligonucleotide sequence, wherein the zip oligonucleotide sequence hybridizes to an antizip oligonucleotide sequence present on either the first or the second reporter components. The kit optionally may comprise a first and a second reporter component and a first and a second target binding component, wherein the zip sequence of the first target binding component hybridizes to the antizip sequence of the first reporter component; and the zip sequence of the second target binding component hybridizes to the antizip sequence of the second reporter component.

The kit optionally comprises a detection component for detecting an epitope formed by the two members of the hemi-peptide pair. In one embodiment, the hemi-peptide pair is one of the pairs in TABLE 4 and the detection reagent is an anti-T7 antibody. In another embodiment, the hemi-peptide pair is one of the pairs in TABLE 5 and the detection reagent is streptactin or strepavidin. In another embodiment, the hemi-peptide pair is one of the pairs in TABLE 6 and the detection reagents are a mutant ribonuclease activated by S-15 and a detectable ribonuclease substrate. In certain embodiments, the kit further comprises an amplification component for producing a plurality of detectable moieties for each molecule of reaction product produced by DCS.

### Example 15

### Detection of EGFR Homodimers in A431 Cells Using NCL

Adherent cultures of A431 cells were serum starved for 16 hours. Cells were detached from the plates with trypsin and the cell suspension treated with AG1478 (1 μM) for 5 minutes at 37° C. and then with EGF (200 ng/mL) for 15 minutes on ice in order to induce receptor dimerization. Cell suspensions were fixed with 2% formaldehyde in PBS on ice for 30 min. The fixed cells were blocked with 50 mM sodium phosphate, pH 6 containing 2% BSA, 5% dextran sulfate, 10 μM tRNA, and 100 μg/mL goat IgG for 1 hour on ice. The cells then were incubated with antibody zipcode conjugates (egfr1-zip2 and egfr1-zip5, 5 μg/mL each) and anti-zicode2_T7_p1 thioester containing the antizip2_{—}10 mer reporter (60 nM) in the blocking buffer for 30 minutes at room temperature. Control samples to assess non-specific signal included one in which both or a single antibody conjugates was omitted. The cells were centrifuged and the supernatant decanted. The cells then were suspended in blocking buffer containing anti-zipcode5_T7_p2Cys containing either the antizip5_{—}10 mer reporter sequence or the antizip5_{—}1 mis reporter sequence and incubated at room temperature for 30 minutes. Intact T7 Cys substituted peptide formed as a product of the DPC reaction was detected by first binding with rabbit anti-T7 antibody conjugated with HRP and then binding with goat anti-rabbit IgG conjugated with Alexa568.

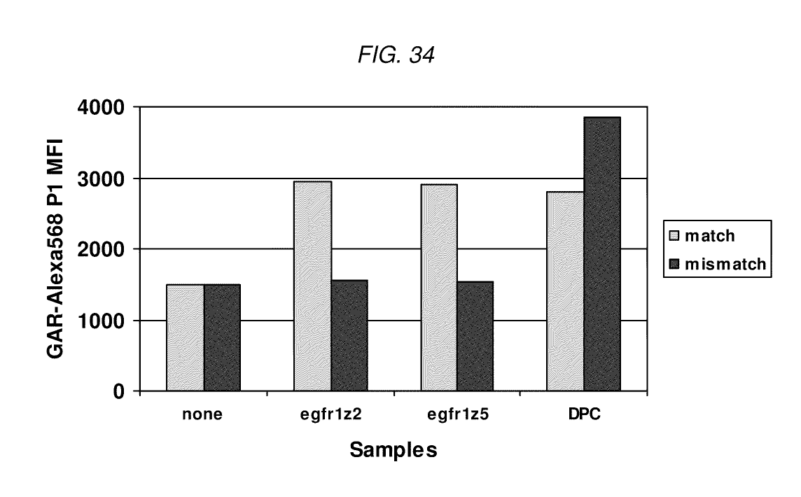

The cells stained with this native chemical ligation DPC assay for EGFR homodimers were analyzed by flow cytometry. The results of the assay are shown in FIG. 34 where the MFI is plotted for each of the control and DPC samples. For samples containing the anti-zip5_T7_p2Cys containing the antizip5_{—}10 mer reporter sequence, the DPC sample containing both antibody conjugates did not produce a signal above the controls containing a single antibody conjugate. In contrast, the cells stained by the DPC reaction with antizip5_T7_p2-Cys containing the antizip5_{—}1 mis reporter sequence yielded a signal that was significantly higher than any of the corresponding control samples.

### Example 16

### Synthesis of T7 Modified Hemipeptides Useful to Form a T7 Peptide Containing An Amide Bond Isostere

### (i) Alternative Synthetic Route for Antizip2 T7 μl S(Et₃MP)

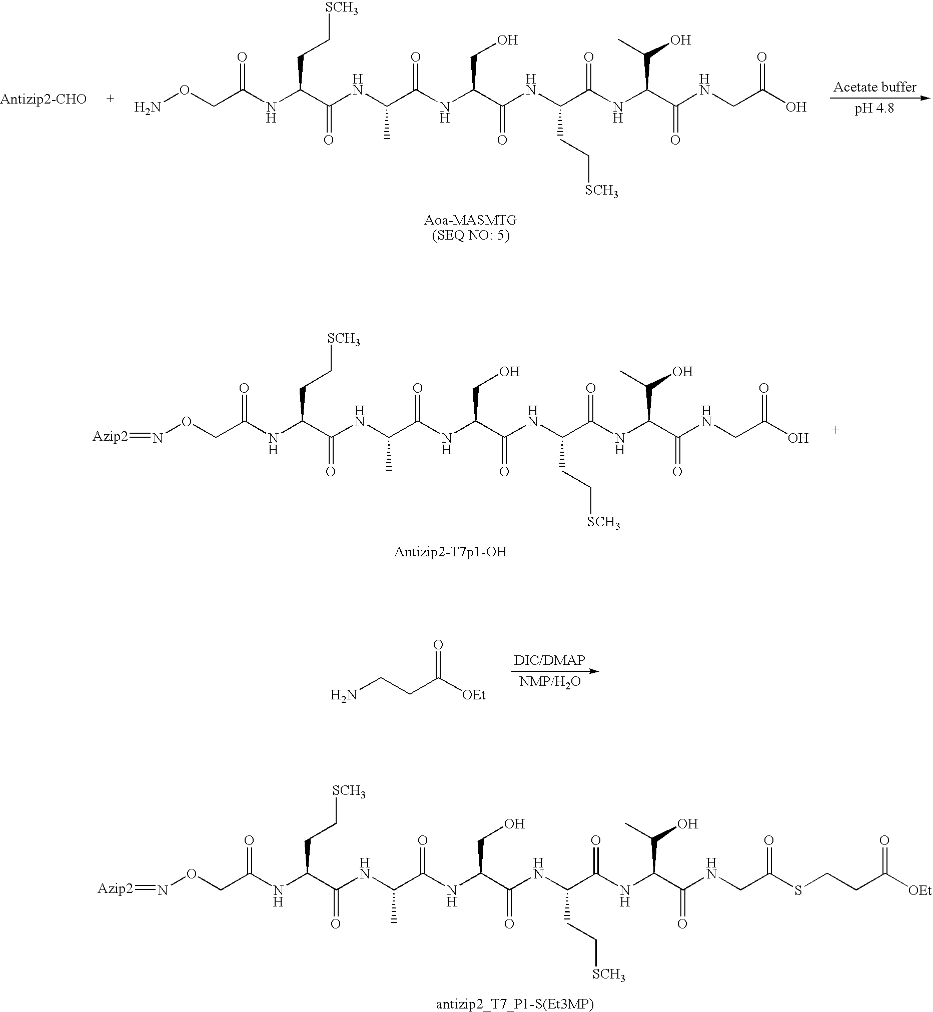

### Antizip2_T7_p1-S(Et3 MP)

Antizip2-T7 p1-OH (10.5 nmol) was dissolved in 5 μL of water and diluted with 45 μL of N-methylpyrrolidone (NMP). The above solution, diisopropylcarbodiimide (5.21 mg, 25 μmol) and 4-dimethylaminopyridine (0.33 mg, 2.7 μmol) dissolved in 20 μL of NMP was added, followed by 5 μL (39 μmol) of ethyl 3-mercaptopropionate. The reaction mixture was agitated for 15 hours at room temperature and quenched with 75 μL of water. The reaction mixture was loaded onto NAPS column (GE healthcare, Piscataway, N.J.). The product was eluted with 700 μL of TEAA buffer (0.05 M, pH 5.5) and purified by HPLC using a TEAA system (Solvent A, 0.05 M TEAA pH 5.5; Solvent B, Acetonitrile; The gradient of solvent B increased from 10% to 40% from 4-14 minutes). The fractions at 12.4 minutes were collected to yield 3.3 nmol (31%) of Antizip2_T7_p1-S(Et3 MP).

(ii) Synthesis of Antizip5-T7 p2-MA

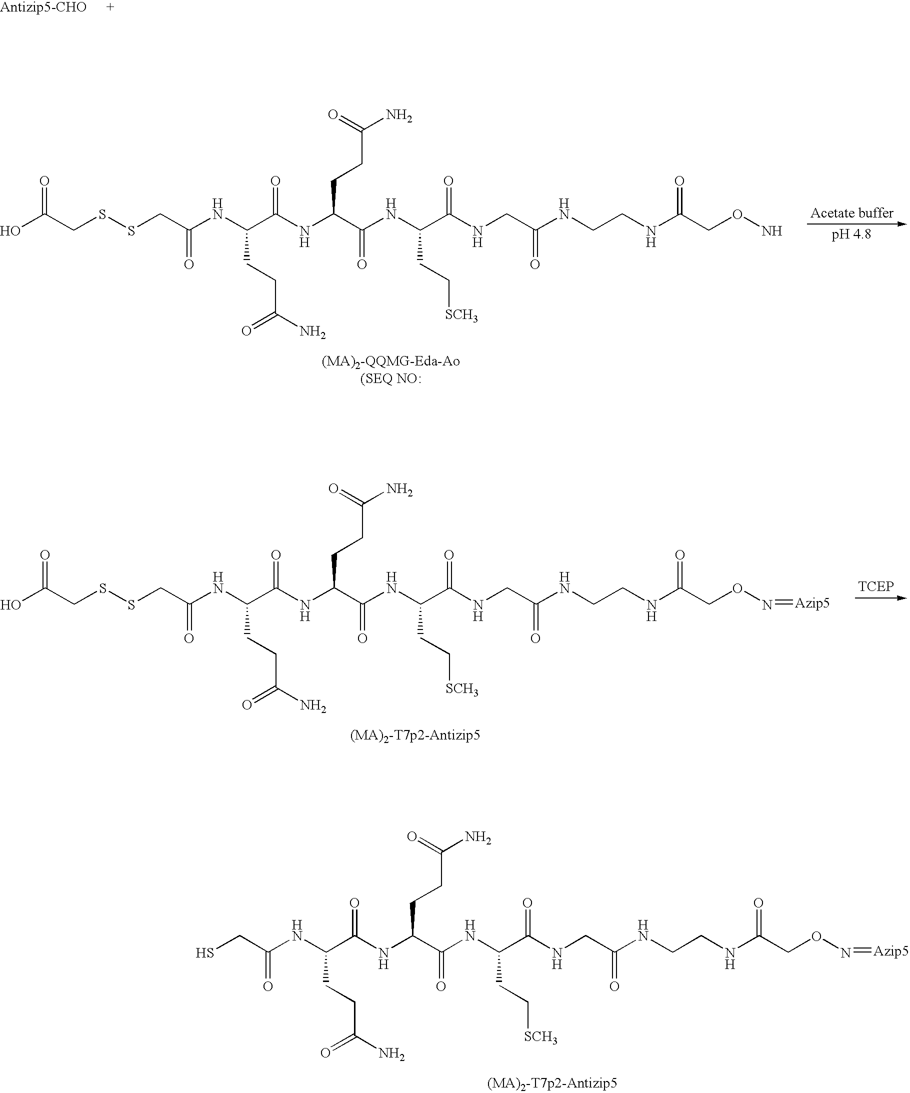

### (MA)₂-QQMG-Eda-Aoa (SEQ NO: 83)

Peptide was synthesized similarly as C-terminal hemipeptide GQQMG (T7-p2) (SEQ NO: 6) except for the last coupling. Dithiodiglycolic acid was used to place the disulfide version of mercapto acetate at the N-terminus instead of glycine. The peptide was cleaved from the resin with TFA/TIS/H₂O (94:3:3) and used without purification.

(MA)₂-T7p2-Antizip5

Antizip5-CHO (20 nmol) and (MA)₂-QQMG-Eda-Aoa (1.12 mg) (SEQ NO: 83) in 100 μL acetate buffer (0.1 M, pH 4.8) was agitated for 4 hours at room temperature. The reaction mixture was diluted with 100 μL of TEAA buffer (0.1 M, pH 7) and loaded onto a NAPS column. The product was eluted out with 700 μL of TEAA buffer and purified by HPLC using TEAA system (Solvent A, 0.1 M TEAA pH 7; Solvent B, Acetonitrile; the gradient of solvent B increased from 10% to 40% from 4-14 minutes). The fraction at 9.8 minutes was collected to yield 12.2 nmol (61%) of Antizip5_T7_p2-(MA)₂.

Preparation of Antizip5_T7_p2-MA stock solution: A solution of TCEP.HCl (1 μL, 100 mM) in water was added to a solution of Antizip5_T7_p2-(MA)₂ (9 μL, 100 μM) in water. The sample solution was agitated for 1 hour at room temperature before use or it was stored at −80° C. for up to 1 month.

Antizip5_T7_p2-MA and Antizip2_T7_p1 thioester were combined to spontaneously produce Antizip2_T7_Thioester_Antizip5, an intact mutant T7 peptide (T7_thioester) linked to two antizip reporter oligonucleotides as shown in the following scheme:

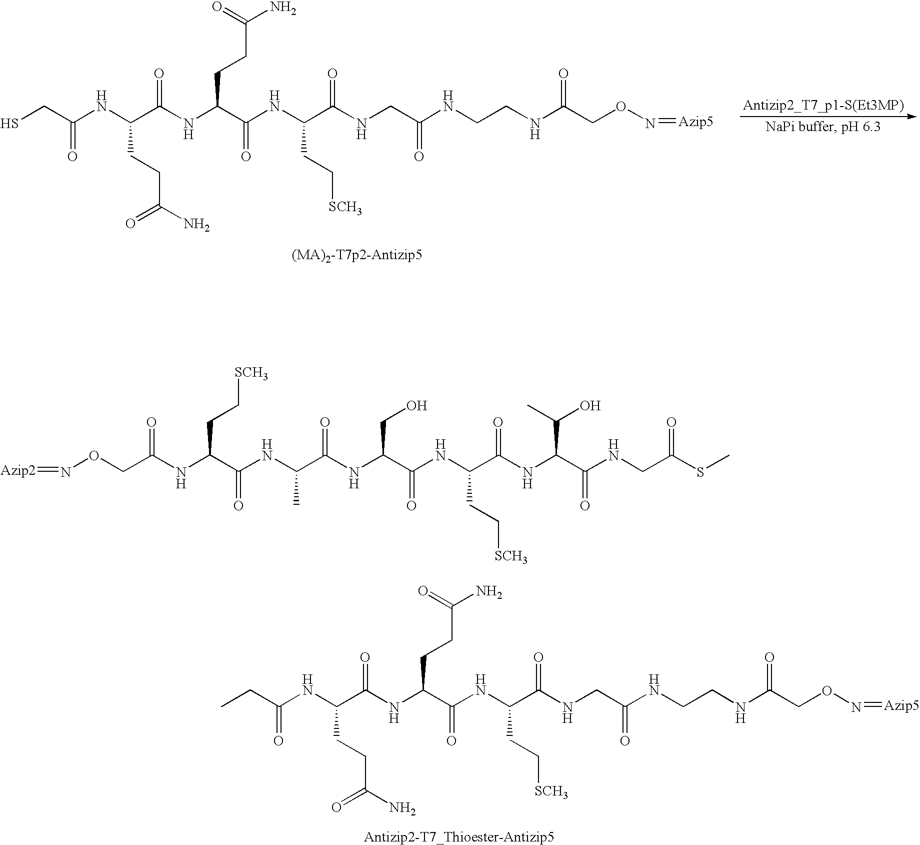

An alternative chemistry has also been employed to produce a T7 peptide containing a thioether bond as an isostere. In this chemistry, one hemipeptide is modified to contain an ethylthiol terminus and the other hemipeptide is modified to contain a haloacetamide moiety. The two hemi-peptides spontaneously react to form a thioether linkage that mimics a Gly-Gly sequence.

### Example 17

### Use of DPC to Detect a DNA Target Through Formation of a T7 Peptide Containing an Amide Bond Isostere

Immobilized DNA target sequences were prepared by incubating immobilized streptavidin in the wells (a solid support) of 96-well microplates (Pierce; Rockford, Ill., BSA blocked) overnight. The wells were washed with 100 μL PBS buffer containing a mixture of 2.5 pMoles each of biotin-zip2 and biotin-zip5. Following incubation, the plates were washed three times with 200 μL of PBS-T, then once with water, and air dried.

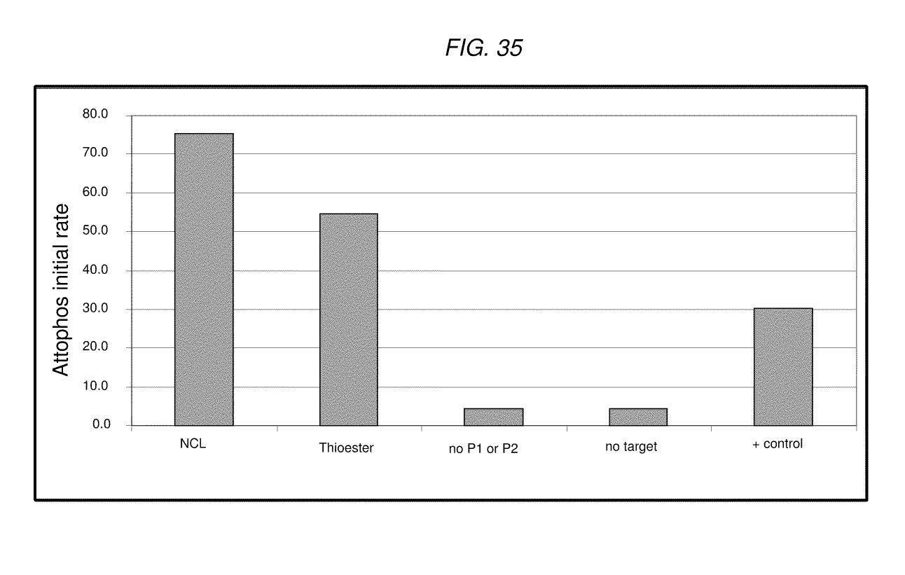

The wells then were incubated with 0.05 μM of Antizip2_T7_p1-S(Et3 MP) in 50 mM sodium phosphate buffer, pH 6.0 containing 5% dextran sulfate for 30 minutes at ambient temperature. The wells then were washed with the sodium phosphate/dextran sulfate buffer and then to the well was added either (MA)₂-T7p2-Antizip5 or Antizip5_T7_p2_Cys in the same buffer. To each well was then added 100 μL of 0.01 μM mouse anti-T7-antibody alkaline phosphatase conjugate (Novagen, Gibbstown, N.J.) in PBS-T buffer. The wells were incubated for 30 minutes at 25° C., and then washed four times with 200 μL PBS-T buffer. The reaction mixtures were developed with Attophos detection solution (Amersham, Piscataway, N.J.) and the kinetics of fluorescence development monitored as described in Example 13. The results are summarized in FIG. 35.

FIG. 35 demonstrates that the resulting T7 thioester peptide (denoted thioester) was formed and detected at a level slightly less than the corresponding T7-Cys peptide (denoted NCL). Negative controls lacking either the target or any of the T7 hemi-peptide antizip molecules demonstrated low background values.

### INCORPORATION BY REFERENCE

The entire disclosure of each of the publications and patent documents referred to herein is incorporated by reference in its entirety for all purposes to the same extent as if each individual publication or patent document were so individually denoted.

### EQUIVALENTS

The invention may be embodied in other specific forms without departing form the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A method of determining the presence and/or amount of a biological target in a sample, the method comprising:
(a) combining the sample with (1) a first probe comprising (i) a first binding moiety with binding affinity to the biological target, (ii) a first oligonucleotide sequence, and (iii) a first product precursor associated with the first oligonucleotide sequence, and (2) a second probe comprising (i) a second binding moiety with binding affinity to the biological target, (ii) a second oligonucleotide sequence capable of hybridizing to the first oligonucleotide sequence, and (iii) a second product precursor associated with the second oligonucleotide sequence, under conditions to permit both the first and second binding moieties to bind to the biological target, if present in the sample, whereupon the first and second oligonucleotide sequences hybridize to one another to bring the first and second product precursors into reactive proximity with one another to produce a reaction product, wherein the reaction product is selected from the group consisting of an intact epitope, an enzyme substrate, an enzyme activator and a ligand; and
(b) exposing the reaction product of step (a), if present, to a detection system comprising an amplification component and a detection component under conditions to permit the production of a plurality of detectable moieties; and
(c) determining the presence and/or amount of the detectable moieties produced in step (b) thereby to determine the presence and/or amount of the biological target in the sample.

2. The method of claim 1, wherein, in step (a), the first binding moiety is covalently associated with the first oligonucleotide sequence.

3. The method of claim 2, wherein, in step (a), the second binding moiety is covalently associated with the second oligonucleotide sequence.

4. The method of claim 1, wherein, in step (a), the first binding moiety is non-covalently associated with the first oligonucleotide sequence.

5. The method of claim 4, wherein the first binding moiety is non-covalently associated with the first oligonucleotide sequence through a zipcode sequence hybridized to an anti-zipcode sequence.

6. The method of claim 4, wherein, in step (a), the second binding moiety is non-covalently associated with the second oligonucleotide sequence.

7. The method of claim 6, wherein the second binding moiety is non-covalently associated with the second oligonucleotide sequence through a zipcode sequence hybridized to an anti-zipcode sequence.

8. A method of determining the presence and/or amount of a biological target in a sample, the method comprising:
(a) providing a first target binding component comprising (i) a first binding moiety having binding affinity to the biological target, and (ii) a first oligonucleotide zipcode sequence;
(b) providing a second target binding component comprising (i) a second binding moiety having binding affinity to the biological target, and (ii) a second oligonucleotide zipcode sequence;
(c) providing a first reporter component comprising (i) a first oligonucleotide anti-zipcode sequence capable of hybridizing to the first oligonucleotide zipcode sequence, (ii) a first reporter oligonucleotide, and (iii) a first product precursor associated with the first reporter oligonucleotide;
(d) providing a second reporter component comprising (i) a second oligonucleotide anti-zipcode sequence capable of hybridizing to the second oligonucleotide zipcode sequence, (ii) a second reporter oligonucleotide capable of hybridizing to the first reporter oligonucleotide sequence, and (iii) a second product precursor associated with the second reporter oligonucleotide sequence and capable of reacting with the first product precursor when brought into reactive proximity with the first product precursor;
(e) combining the sample with the first target binding component, the second target binding component, the first reporter component, and the second reporter component under conditions so that the first and second binding moieties bind to the biological target, if present in the sample, whereupon (i) the first zipcode sequence hybridizes to the first anti-zipcode oligonucleotide sequence, (ii) the second oligonucleotide zipcode sequence hybridizes to the second oligonucleotide anti-zipcode sequence, and (iii) the second reporter oligonucleotide hybridizes to the first reporter oligonucleotide to bring the first and second product precursors into reactive proximity to produce a reaction product;
(f) exposing the reaction product of step (e), if present, to a detection system comprising an amplification component and a detection component under conditions to permit the production of a plurality of detectable moieties; and
(g) determining the presence and/or amount of the detectable moieties thereby to determine the presence and/or amount of the biological target in the sample.

9. The method of claim 8, wherein in step (e) the first target binding component, the second target binding component, the first reporter component, and the second reporter component are all combined with the sample at the same time.

10. The method of claim 8, wherein in step (e) the first target binding component and the second target binding component are added to the sample before the first reporter component and the second reporter component.

11. The method of claim 1, wherein the amplification component comprises an enzyme that catalyzes the production of the detectable moieties.

12. The method of claim 1, wherein the amplification component is capable of producing at least 10 molecules of the detectable moieties per molecule of the reaction product.

13. The method of claim 12, wherein the amplification component is capable of producing at least 100 molecules of the detectable moieties per molecule of the reaction product.

14. The method of claim 13, wherein the amplification component is capable of producing at least 1,000 molecules of the detectable moieties per molecule of the reaction product.

15. The method of claim 1, wherein reaction product is a peptide or protein.

16. The method of claim 15, wherein the reaction product comprises at peptidyl sequence selected from the peptides listed in FIG. 15.

17. The method of claim 1, wherein the reaction product is a small molecule.

18. (canceled)

19. The method of claim 1, wherein the biological target is a protein, peptide, immunoglobulin, growth factor receptor or enzyme.

20. The method of claim 1, wherein the biological target is a homodimeric protein.

21. The method of claim 1, wherein the biological target is a heterodimeric protein.

22. The method of claim 1, wherein the biological target is selected from the group consisting of a Bcr-Abl heterodimer, an ErbB family homodimer, an ErbB family heterodimer, and PDGF.

23-35. (canceled)

36. A method of determining the presence and/or amount of a biological target in a sample, the method comprising:
(a) combining the sample with (1) a first probe comprising (i) a first binding moiety with binding affinity to the biological target, (ii) a first oligonucleotide sequence, and (iii) a first masked product precursor associated with the first oligonucleotide sequence and (2) a second probe comprising (i) a second binding moiety with binding affinity to the biological target, (ii) a second oligonucleotide sequence capable of hybridizing to the first oligonucleotide sequence, and (iii) an unmasking group associated with the second oligonucleotide sequence, under conditions to permit the first and second binding moieties to bind to the biological target, if present in the sample, whereupon the first and second oligonucleotide sequences hybridize to one another to bring the unmasking group into reactive proximity with the masked product precursor to produce an unmasked reaction product;
(b) exposing the reaction product of step (a), if present, to a detection system comprising an amplification component and a detection component under conditions to permit the production of a plurality of detectable moieties; and
(c) determining the presence and/or amount of the detectable moieties thereby to determine the presence and/or amount of the biological target in the sample.

37-51. (canceled)

52. A kit comprising:
(a) a first probe comprising (i) a first binding moiety with binding affinity to a biological target, (ii) a first reporter oligonucleotide sequence associated with the first binding moiety, and (iii) a first product precursor associated with the first reporter oligonucleotide sequence;
(b) a second probe comprising (i) a second binding moiety with binding affinity to the biological target, (ii) a second reporter oligonucleotide sequence associated with the second binding moiety, and (iii) a second product precursor associated with the second reporter oligonucleotide sequence,
wherein upon the binding of the first and second binding moieties to the biological target the first and second reporter oligonucleotide sequences are capable of hybridizing to one another and the first and second product precursors are capable of reacting with one another to produce a reaction product selected from the group consisting of an intact epitope, an enzyme substrate, an enzyme activator, and a ligand;
(c) a detection system comprising an amplification component and a detection component capable of producing a plurality of detectable moieties; and
(d) instructions for using the kit for detecting the biological target.

53. (canceled)

54. A kit comprising:
(a) a first target binding component comprising (i) a first binding moiety having binding affinity to the biological target, and (ii) a first oligonucleotide zipcode sequence associated with the first binding moiety;
(b) a second target binding component comprising (i) a second binding moiety having binding affinity to the biological target, and (ii) a second oligonucleotide zipcode sequence associated with the second binding moiety;
(c) a first reporter component comprising (i) a first oligonucleotide anti-zipcode sequence capable of hybridizing to the first oligonucleotide zipcode sequence, (ii) a first reporter oligonucleotide associated with the first oligonucleotide zipcode sequence, and (iii) a first product precursor associated with the first reporter oligonucleotide sequence;
(d) a second reporter component comprising (i) a second oligonucleotide anti-zipcode sequence capable of hybridizing to the second oligonucleotide zipcode sequence, (ii) a second reporter oligonucleotide associated with the second oligonucleotide zipcode sequence and capable of hybridizing to the first reporter oligonucleotide sequence, and (iii) a second product precursor associated with the second reporter oligonucleotide sequence,
wherein upon the binding of the first and second binding moieties to the biological target, hybridization of the first zipcode and anti-zipcode oligonucleotide sequences and hybridization of the second zipcode and anti-zipcode oligonucleotide sequences, the first and second reporter oligonucleotide sequences hybridize to one another to bring the first and second product precursors into reactive proximity to produce a reaction product selected the group consisting of an intact epitope, an enzyme substrate, an enzyme activator, and a ligand;
(e) a detection system comprising an amplification component and a detection component capable of producing a plurality of detectable moieties; and
(f) instructions for using the kit for detecting the biological target.

55-64. (canceled)
